SEQUENCE LISTING (16 sequences)

SEQ ID NO: 1 - protein, 112 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 2 - protein, 115 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 3 - protein, 219 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 444 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 16 aa, Artificial Sequence
  Synthetic construct
KSSQSLLYSRGKTYLN

SEQ ID NO: 6 - protein, 7 aa, Artificial sequence
  Synthetic construct
AVSKLDS

SEQ ID NO: 7 - protein, 9 aa, Artificial sequence
  Synthetic construct
VQGTHYPFT

SEQ ID NO: 8 - protein, 10 aa, Artificial sequence
  Synthetic construct
GYDFTRYYIN

SEQ ID NO: 9 - protein, 17 aa, Artificial sequence
  Synthetic construct
WINPGSGNTKYNEKFKG

SEQ ID NO: 10 - protein, 6 aa, Artificial Sequence
  Synthetic construct
EGITVY

SEQ ID NO: 11 - DNA, 336 bp, Artificial Sequence
  Synthetic construct DNA sequence of SEQ ID NO. 1
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaa

SEQ ID NO: 12 - DNA, 345 bp, Artificial Sequence
  Synthetic construct DNA sequence of SEQ ID NO. 2
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctca

SEQ ID NO: 13 - DNA, 657 bp, Artificial Sequence
  Synthetic construct DNA sequence of SEQ ID NO. 3
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 14 - DNA, 1332 bp, Artificial Sequence
  Synthetic construct DNA sequence of SEQ ID NO. 4
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 15 - protein, 219 aa, Artificial Sequence
  Synthetic Construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 442 aa, Artificial Sequence
  Synthetic Construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0010713 A1
(43) Pub. Date: Jan. 11, 2024

(54) ANTI-N3pGlu AMYLOID BETA ANTIBODIES AND USES THEREOF

(71) Applicant: Eli Lilly and Company, Indianapolis, IN (US)

(72) Inventors: Adam S. FLEISHER, Indianapolis, IN (US); Mark MINTUN, Indianapolis, IN (US); John Randall SIMS, II, Zionsville, IN (US); JonDavid SPARKS, Fishers, IN (US)

(21) Appl. No.: 18/260,125

(22) PCT Filed: Jan. 11, 2022
(86) PCT No.: PCT/US2022/011894

§ 371 Date: Jun. 30, 2023

Related U.S. Application Data
(60) Provisional application No. 63/296,694, filed on Jan. 5, 2022.
(60) Provisional application No. 63/284,248, filed on Nov. 30, 2021.
(60) Provisional application No. 63/277,298, filed on Nov. 9, 2021.
(60) Provisional application No. 63/227,054, filed on Jul. 29, 2021.
(60) Provisional application No. 63/192,262, filed on May 24, 2021.
(60) Provisional application No. 63/160,380, filed on Mar. 12, 2021.
(60) Provisional application No. 63/155,029, filed on Mar. 1, 2021.
(60) Provisional application No. 63/135,932, filed on Jan. 11, 2021.

(51) Int. Cl. C07K 16/18; A61P 25/28

(52) U.S. Cl. CPC C07K 16/18; A61P 25/28; A61K 2039/505

(57) ABSTRACT

The invention is directed to treatment or prevention of a disease characterized by deposition of Aβ in the brain using anti-N3pGlu Aβ antibodies. The diseases that can be treated or prevented include, e.g., Alzheimer's disease, Down's syndrome, and cerebral amyloid angiopathy. The invention, in some aspects, is related to doses and dosing regimens useful for such treatments. The invention is also related to, in some aspects, human subjects who are responsive to treatment or prevention of a disease characterized by deposition of Aβ in the brain using anti-N3pGlu Aβ antibodies. The invention is also related to human subject who have one or two alleles of APOE4.

The present disclosure is related to methods of preventing or treating a disease with anti-N3pGlu Aβ antibodies, wherein the disease is characterized by deposition of amyloid beta (Aβ) in a subject. The present disclosure is also related to doses and dosing regimens of the anti-N3pGlu Aβ antibodies useful for treating or preventing a disease characterized by deposition of Aβ. Some aspects of the present disclosure are related to treating or preventing a disease characterized by deposition of Aβ in subjects, wherein the subjects are selected based on i) their tau level/burden in the whole brain (global tau), ii) their tau level/burden in portions of the brain (e.g., in different lobes of the brain), and/or iii) the presence of one or two alleles of APOE4 in the subject's genome. The diseases that can be treated or prevented using antibodies, dosing regimens, or methods disclosed herein include, e.g., Alzheimer's disease (AD), Down's syndrome, and cerebral amyloid angiopathy (CAA). The present disclosure is also related to slowing disease progression in subjects with early symptomatic Alzheimer's disease, optionally, in the presence of intermediate brain tau burden. The present disclosure is also related to slowing disease progression of AD. Treatment with the anti-N3pG Aβ antibodies of the present disclosure could be initiated in patients with evidence of AD neuropathology and mild cognitive impairment or mild dementia stage of disease, optionally in the presence of brain tau load. In some embodiments, the brain tau load is very low, low, intermediate, or high tau.

A cure for AD is one of the most significant unmet need of society. Accumulation of amyloid-β peptide in the form of brain amyloid plaques is an early and essential event in Alzheimer's disease leading to neurodegeneration and consequently the onset of clinical symptoms, such as, cognitive and functional impairment (Selkoe, “The Origins of Alzheimer Disease: A is for Amyloid,” JAMA 283:1615-7 (2000); Hardy et al., “The Amyloid Hypothesis of Alzheimer's Disease: Progress and Problems on the Road to Therapeutics,” Science 297:353-6 (2002); Masters et al., “Alzheimer's Disease,” Nat. Rev. Dis. Primers 1:15056 (2015); and Selkoe et al., “The Amyloid Hypothesis of Alzheimer's Disease at 25 years,” EMBO Mol. Med. 8:595-608 (2016)).

Amyloid beta is formed by the proteolytic cleavage of a larger glycoprotein called amyloid precursor protein (APP). APP is an integral membrane protein expressed in many tissues, but especially in neuron synapses. APP is cleaved by 7-secretase to release the Aβ peptide, which encompasses a group of peptides ranging in size from 37-49 amino acid residues. Aβ monomers aggregate into various types of higher order structures including oligomers, protofibrils, and amyloid fibrils. Amyloid oligomers are soluble and may spread throughout the brain, while amyloid fibrils are larger and insoluble and can further aggregate to form amyloid plaques. The amyloid plaques found in human patients include a heterogeneous mixture of Aβ peptides, some of which include N-terminal truncations and further may include N-terminal modifications such as an N-terminal pyroglutamate residue (pGlu).

The role for amyloid plaques in driving disease progression is supported by study of uncommon genetic variants that either increase or decrease Aβ deposition (Fleisher et al., “Associations Between Biomarkers and Age in the Presenilin 1 E280A Autosomal Dominant Alzheimer Disease Kindred: A Cross-sectional Study,” JAMA Neurol 72:316-24 (2015); Jonsson et al., “A Mutation in APP Protects Against Alzheimer's Disease and Age-related Cognitive Decline,” Nature 488:96-9 (2012)). In addition, presence of amyloid plaques early in the disease increases the likelihood of progression of mild cognitive impairment (MCI) to AD dementia (Doraiswamy et al., “Amyloid-β Assessed by Florbetapir F18 PET and 18-month Cognitive Decline: A Multicenter Study,” Neurology 79:1636-44 (2012)). Interventions or therapies aiming at removal of Aβ plaques are hypothesized to slow the clinical progression of AD.

Some known anti-Aβ antibodies include bapineuzumab, gantenerumab, aducanumab, GSK933776, solanezumab, crenezumab, ponezumab, and lecanemab (BAN2401). Antibodies targeting Aβ have shown promise as a therapeutic for Alzheimer's disease in both preclinical and clinical studies. Despite this promise, many antibodies targeting amyloid have failed to meet therapeutic endpoints in multiple clinical trials. The history of anti-amyloid clinical trials spans almost two decades and has, for the most part, cast doubt on the potential of such therapies to effectively treat AD (Aisen et al., “The Future of Anti-amyloid Trials,” The Journal of Prevention of Alzheimer's Disease 7:146-151 (2020), Budd et al., “Clinical Development of Aducanumab, an Anti-Aβ Human Monoclonal Antibody Being Investigated for the Treatment of Early Alzheimer's Disease,” The Journal of Prevention of Alzheimer's Disease 4(4):255-263 (2017) and Klein et al., “Gantenerumab Reduces Amyloid-β Plaques in Patients with Prodromal to Moderate Alzheimer's Disease: A PET Substudy Interim Analysis,” Alzheimer's Research & Therapy 11.1: 1-12 (2019)).

Amyloid plaques found in human patients include a heterogeneous mixture of Aβ peptides. N3pGlu Aβ (also referred to as N3pG Aβ, N3pE Aβ, Aβ pE3-42, or Aβp3-42) is a truncated form of Aβ peptide and is found only in amyloid plaques. N3pGlu Aβ lacks the first two amino acid residues at the N-terminus of human Aβ and has a pyroglutamate which is derived from glutamic acid at the third amino acid position of Aβ. Although N3pGlu Aβ peptide is a minor component of the deposited Aβ in the brain, studies suggest that N3pGlu Aβ peptide has aggressive aggregation properties and accumulates early in the deposition cascade. Passive immunization by long term chronic administration of antibodies against Aβ found in plaques, including N3pGlu Aβ, has been shown to disrupt the Aβ aggregates and promote the clearance of plaques in the brain in various animal models.

Antibodies to N3pGlu Aβ are known in the art. For example, U.S. Pat. No. 8,679,498 (which is hereby incorporated by reference in its entirety, including the anti-N3pGlu Aβ antibodies disclosed therein) discloses anti-N3pGlu Aβ antibodies and methods of treating diseases, such as, Alzheimer's disease, with these antibodies.

Donanemab (disclosed in U.S. Pat. No. 8,679,498) is an antibody directed at the pyroglutamate modification of the third amino acid of amyloid beta (N3pGlu Aβ) epitope that is present only in brain amyloid plaques. The mechanism of action of donanemab is the targeting and removal of existing amyloid plaque, which is a key pathological hallmark of AD. A second neuropathological hallmark of AD is the presence of intracellular neurofibrillary tangles containing hyperphosphorylated tau protein. It is possible that Aβ triggers tau pathology, with a more complex and synergistic interaction between Aβ and tau manifesting at later stages and driving disease progression (Busche et al., “Synergy Between Amyloid-β and Tau in Alzheimer's disease,” Nature Neuroscience 23:1183-93 (2020)).

The treatment and prevention strategy for donanemab includes targeting N3pGlu Aβ specific to amyloid plaque in, e.g., early symptomatic AD patients with existing brain amyloid load. This rationale is based on the amyloid hypothesis of AD, which states that the production and deposition of Aβ is an early and necessary event in the pathogenesis of AD. See, e.g., Selkoe, “The Origins of Alzheimer Disease: A is for Amyloid,” JAMA 283:1615-1617 (2000). Clinical support for this hypothesis comes from the demonstration that parenchymal Aβ levels are elevated before the appearance of symptoms of AD and supported by genetic variants of AD that overproduce brain Aβ and genetic variants that protect against Aβ production. See, e.g., Jonsson et al., “A Mutation in APP Protects Against Alzheimer's Disease and Age-related Cognitive Decline,” Nature 488 (7409):96-99 (2012) and Fleisher et al., “Associations Between Biomarkers and Age in the Presenilin 1 E280A Autosomal Dominant Alzheimer Disease Kindred: A Cross-sectional Study,” JAMA Neurol. 72:316-24 (2015).

However, significant problems exist with long term chronic administration of Aβ antibodies. Administration of Aβ antibodies has led to adverse events in humans, such as, amyloid-related imaging abnormalities (ARIA), suggestive of vasogenic edema and sulcal effusions (ARIA-E), microhemorrhages and haemosiderin deposits (ARIA-H), infusion site reactions, and risk of immunogenicity. See, e.g., Piazza and Winblad, “Amyloid-Related Imaging Abnormalities (ARIA) in Immunotherapy Trials for Alzheimer's Disease: Need for Prognostic Biomarkers?” Journal of Alzheimer's Disease, 52:417-420 (2016); Sperling, et al., “Amyloid-related Imaging Abnormalities in Patients with Alzheimer's Disease Treated with Bapineuzumab: A Retrospective Analysis,” The Lancet Neurology 11.3: 241-249 (2012); Brashear et al., “Clinical Evaluation of Amyloid-related Imaging Abnormalities in Bapineuzumab Phase III Studies,” J. of Alzheimer's Disease 66.4:1409-1424 (2018); Budd et al., “Clinical Development of Aducanumab, an Anti-Aβ Human Monoclonal Antibody Being Investigated for the Treatment of Early Alzheimer's Disease,” The Journal of Prevention of Alzheimer's Disease 4.4: 255 (2017).

Although the exact cause of such adverse events is not known, it is generally believed that antibody treatment disrupts blood-brain barrier through interaction with the cerebral vascular amyloid and that this disruption leads to a leaky barrier and the manifestation of edema in patients. Several possible mechanisms of action have been postulated, e.g., that removal of amyloid from the vessel wall destabilizes the neurovascular unit, localized inflammation/infiltrates in the neurovascular unit, increased levels of cerebral vascular amyloid due to higher levels of interstitial soluble Aβ in response to parenchymal plaque clearance or altered localization of AQP-4 in astrocytic end feet projections in the neurovascular unit.

Several therapeutic amyloid targeted antibodies have demonstrated dose-response related increases in ARIA-E. See, e.g., Brashear et al., “Clinical Evaluation of Amyloid-related Imaging Abnormalities in Bapineuzumab Phase III Studies,” J. of Alzheimer's Disease 66.4:1409-1424 (2018); Budd et al., “Clinical Development of Aducanumab, an Anti-Aβ Human Monoclonal Antibody Being Investigated for the Treatment of Early Alzheimer's Disease,” The Journal of Prevention of Alzheimer's Disease 4.4: 255 (2017). In some instances, there is a higher incidence rate of ARIA-E in patients harboring the epsilon-4 allele of apolipoprotein E (referred to herein as APOE4, apoE4, or ApoE-ε4).

To decrease the rate of ARIA-E adverse events while maintaining plaque clearance, some antibody treatment programs implement dose-titration schemes that included multiple dose escalations (3-4 steps) over a period of ˜6-months prior to reaching their efficacious dose level. See, e.g., Budd et al., “Clinical Development of Aducanumab, an Anti-Aβ Human Monoclonal Antibody Being Investigated for the Treatment of Early Alzheimer's Disease,” The Journal of Prevention of Alzheimer's Disease 4.4:255 (2017) and Klein et al., “Gantenerumab Reduces Amyloid-β Plaques in Patients with Prodromal to Moderate Alzheimer's Disease: a PET Substudy Interim Analysis,” Alzheimer's Research & Therapy 11.1:101 (2019). Such treatment regimens may not fully clear amyloid plaques or may delay clearance of amyloid plaque.

Thus, a need exists for improved doses, dosing regimens, or methods that properly treat subjects without causing or increasing problematic adverse events.

One aspect of the present disclosure provides for doses and dosing regimens that circumvent problematic adverse events, such as ARIA with vasogenic edema, that have been observed in patients receiving therapeutic antibodies that bind to deposited amyloid and have been dose limiting for some clinical development programs.

The antibodies of the present disclosure bind selectively to N3pGlu Aβ found primarily in deposited amyloid plaque. The prevalence of the N3pGlu Aβ peptide in deposited parenchymal plaque is very low relative to other Aβ peptide species (˜1 to 2%) where the majority is full-length Aβ₁₋₄₂. Thus, the total number of binding sites for the antibodies of the present disclosure relative to other plaque binding Aβ antibodies is dramatically lower. Biochemical analysis of CAA, the amyloid depositing along CNS blood vessels, demonstrated a similar low prevalence of N3pGlu peptides (˜2%).

Surprisingly, the antibodies of the present disclosure do not require multiple dose escalations over a long duration. In some instances, the antibodies may reach efficacious dose level without causing higher incidence rates of adverse events. Moreover, in some instances, the anti-N3pGlu Aβ antibodies of the present disclosure and their dosing regimen, as described herein, facilitate rapid brain amyloid clearance while minimizing incidence and/or severity of the ARIA adverse events observed with anti-amyloid antibodies.

The beneficial effect of the present improved doses, dosing regimens, and methods of the present disclosure could be, e.g., because the antibodies quickly clear parenchymal plaque while having lower total binding to vascular amyloid (e.g., due to the lower prevalence of the N3pGlu peptide). In other words, the beneficial effect of the present improved doses, dosing regimens, and methods of the present disclosure may be due to a combination of i) their ability to target parenchymal/vascular plaque and achieve fast amyloid plaque lowering and ii) the relative paucity of antibody binding sites found in both parenchymal and vascular amyloid deposits. Clinical studies have demonstrated that the treatment of Alzheimer's patients using the present improved doses, dosing regimens, and methods of the present disclosure resulted in rapid clearance of deposited amyloid (such as amyloid plaques) from the brain of subjects. The rate of amyloid clearance was significantly faster than published data (Budd et al., The Journal of Prevention of Alzheimer's Disease 4.4:255 (2017) and Klein et al., Alzheimer's Research & Therapy 11.1:101 (2019)) from other amyloid targeting therapeutic antibodies at a range of doses, despite sparse prevalence of target epitope of the antibodies of the present disclosure.

The dosing regimens described herein facilitate the antibodies of the present disclosure to rapidly remove brain amyloid while minimizing incidence and/or severity of the ARIA adverse events observed with this class of therapeutic antibodies. Moreover, the dosing regimen, as disclosed herein, provides high amount of amyloid removal early (e.g., about 60% of the subjects have an ‘amyloid negative’ scan by 52 weeks). The dosing schemes described herein facilitate the anti-N3pGlu Aβ antibody to quickly remove parenchymal plaque while having lower total binding to vascular amyloid (due to the lower prevalence of the N3pGlu peptide).

As mentioned above, antibodies targeting amyloid plaques, such as antibodies targeting Aβ, have shown promise as a therapeutic for Alzheimer's disease in both preclinical and clinical studies. Despite this promise, antibodies targeting amyloid have failed to meet therapeutic endpoints in multiple clinical trials. The history of anti-amyloid clinical trials spans almost two decades and has, for the most part, cast doubt on the potential of such therapies to effectively treat AD (Aisen et al., “The Future of Anti-amyloid Trials,” The Journal of Prevention of Alzheimer's Disease 7 146-151 (2020)). To date, only a handful of AD treatments have been approved. A challenge in treating Alzheimer's disease is that it is still principally diagnosed and treated based on symptoms, e.g., like a psychiatric illness, rather than based on brain pathology. Yet another challenge is a replication crisis faced during clinical trials where it is often difficult to obtain replicable results even if clinical trials are designed nearly identically. This is caused by two main factors. First, most trials set enrollment criteria based on symptoms rather than pathology. Thus, they end up enrolling a heterogenous population with wide variation in levels of underlying pathology or worse, patients with different underlying diseases. Accordingly, AD in these patients progresses at very different rates, and intra-group variability, measured by standard deviation of the mean, for example, is quite large in AD trials. Also, the population heterogeneity problem is compounded by intra-subject noise in the outcome measurements.

Determining whether subjects having Aβ plaques are going to respond to an anti-N3pGlu Aβ antibody treatment is uniquely challenging. This is partly because of the physiological and clinical heterogeneity amongst the subjects suffering from Aβ plaques and because subjects are still principally being diagnosed on their symptoms. For example, determining if a patient with subtle cognitive symptoms, such as memory decline, suffers from prodromal or preclinical Alzheimer's disease and may progress to AD dementia within the near future remains a challenge for clinicians.

AD clinical trial placebo populations vary widely in trajectories of cognitive and functional decline (Veitch et al., “Understanding Disease Progression and Improving Alzheimer's Disease Clinical Trials: Recent Highlights from the Alzheimer's Disease Neuroimaging Initiative,” Alzheimer's & Dementia 15.1: 106-152 (2019)), which is believed to be due to heterogeneity in trial populations (Devi et al., “Heterogeneity of Alzheimer's Disease: Consequence for Drug Trials?” Alzheimer's Research & Therapy 10.1: 1-3 (2018)). This amplifies the problems of identifying and treating subjects who may benefit from a particular treatment. The task of properly identifying whether a patient may respond to anti-N3pGlu Aβ antibody treatments is of utmost importance for, e.g., a timely referral to a memory clinic, a correct and early AD diagnosis, initiation of symptomatic treatment, future planning, and initiating disease-modifying treatments.

Historically, trial cohorts have been selected by clinical features such as cognitive test score ranges and self-reported problems with memory. After years of failures, experts in the field have advocated testing anti-amyloid disease modifying therapies (DMTs) earlier in the course of the disease (Aisen et al. 2020). However, several clinical studies for anti-amyloid DMTs have failed to meet their endpoints despite targeting patients in early stages of Alzheimer's disease. For instance, phase III clinical trial (Cread trial) for Crenezumab recruited patients with prodromal-to-mild AD. The results for this study were exclusively negative. No difference was found for both endpoints—primary and secondary—between the treatment versus placebo groups or within the prodromal versus mild AD subgroups (NCT03114657 at clinicaltrials.gov; Therapeutics: Crenezumab. Alzforum. AC Immune SA, Genentech, Hoffmann-La Roche; 2019 [cited 2020 Sep. 7]. Available from: alzforum.org/therapeutics/crenezumab). Similarly, a phase II/III clinical trial (SCarlet RoAD trial) assessing the efficacy and safety of gantenerumab in prodromal AD patients was terminated because the probability of obtaining efficacy on primary and secondary endpoints in the trial was low (Ostrowitzki et al., “A Phase III Randomized Trial of Gantenerumab in Prodromal Alzheimer's Disease,” Alzheimer's research & therapy 9.1: 1-15 (2017)).

Thus, a need exists for improved methods that properly identify whether a subject is going to respond to amyloid targeting therapeutics.

Doody et al., “Phase 3 Trials of Solanezumab for Mild-to-Moderate Alzheimer's Disease,” NEJM, 370; 4, 311-321 (2014) indicate that “[n]o clear differential treatment effects on efficacy measures were observed between APOE ε4 carriers and noncarriers.” It has now been found that administering an anti-N3pGlu Aβ antibody to a human subject that has one or two alleles of APOE4 (e.g., a heterozygous or a homozygous carrier of APOE4) provides unexpected and surprising efficacy when compared to non-carriers of one or more of those alleles. Thus, some of the embodiments of the present disclosure involve administering doses of anti-N3pGlu Aβ antibodies to patients who have that allele as a means of slowing the cognitive decline of those patients. Specifically, it has been found that there is a greater effect in carriers of APOE4 than in non-carriers when the patients are administered anti-N3pGlu Aβ antibodies. This means that the patients administered anti-N3pGlu Aβ antibodies that have APOE4 exhibit less cognitive decline than non-carriers, when measured using various clinical measurements and at various endpoints.

Across all therapeutic clinical trials selected for the presence of amyloid pathology, at baseline, carriers are younger and have higher amyloid loads and higher tau pathology. The clinical decline across all scales for the placebo groups does not differ by carrier status. For placebo groups, comparison of carrier status shows there is no significant longitudinal change in amyloid, however there is a trend toward greater tau change in carriers vs. non-carriers. The relative longitudinal change of amyloid while on therapy shows a greater decrease in non-carriers than carriers. One hypothesis to consider is the interaction of APOE with tau. It has been known that amyloid deposition concentrates and contains APOE embedded within the plaque. More recently, it was shown that APOE is also isolated with tau tangles. Animal data further suggest that there is an APOE interaction with tau. In addition, a rare mutation in APOE appeared to protect a subject, carrying an autosomal dominant PSEN1 mutation, well beyond the age of typical onset despite having substantial brain amyloid load but relatively low tau burden. In some embodiments, the present disclosure shows that APOE also influences tau beyond an amyloid interaction and that the rate of tau change may be faster in carriers. Furthermore, the impact of treatment may have a greater influence on tau progression which is more directly linked to clinical progression. Tau progression and spread has been linked to Low density lipoprotein receptor-related protein 1 (LRP1, also known as alpha-2-macroglobulin receptor, apolipoprotein E receptor or cluster of differentiation 91). See Rauch et al., “LRP1 is a Master Regulator of Tau Uptake and Spread,” Nature 580 (7803):381-385 (2020), which is hereby incorporated by reference in its entirety. Recently reported, LRP1 appears to facilitate tau internalization and degradation via an APOE mediated mechanism. See Cooper et al., “Regulation of Tau Internalization, Degradation, and Seeding by LRP1 Reveals Multiple Pathways for Tau Catabolism,” Journal of Biological Chemistry 100715 (2021), which is hereby incorporated by reference in its entirety.

One aspect of the present disclosure is based on the discovery that Alzheimer's patients with low or moderate tau, very low to moderate tau, or not having high tau are responsive to treatment with anti-N3pGlu Aβ antibodies, and patients having high tau levels, even if clinically classified as preclinical or early-stage AD, may not be as effectively treated with anti-N3pGlu Aβ antibodies. Another aspect of the present disclosure is based on the discovery that Alzheimer's patients having one or two alleles of APOE4 are responsive to treatment with anti-N3pGlu Aβ antibodies. Yet another aspect of the present disclosure is based on the discovery that Alzheimer's patients having one or two alleles of APOE4 and low or moderate tau, very low to moderate tau, or not having high tau are responsive to treatment with anti-N3pGlu Aβ antibodies.

Identifying the subjects that are most responsive to treatment with an anti-N3pGlu Aβ antibody solves the 20+ year old problem of finding a clinically effective anti-amyloid treatment and reflects a significant advance in the art. Some aspects of the present disclosure are directed to diagnosing and treating patients based on their brain pathology. Selecting patients based on their brain pathology not only provides a more homogenous population in clinical trials and decreases noise to ensure highly replicable results but it also ensures proper identification of the stage of AD and its progression. Proper identification of the stage of AD allows, e.g., for a timely referral to a memory clinic, a correct and early AD diagnosis, initiation of symptomatic treatment, future planning, and initiating disease-modifying treatments.

Some aspects of the present disclosure provide for a dosing regimen where a human subject, suffering from a disease characterized by Aβ plaques in their brain, is administered an anti-N3pGlu Aβ antibody in two steps. In a first step, the human subject is administered one or more first doses (or low doses) of about 100 mg to about 700 mg of the anti-N3pGlu Aβ antibody, wherein each first dose (low dose) is administered once about every 4 weeks (i.e., at a frequency of once every four weeks). About four weeks after administering the one or more first doses, the human subject is administered one or more second doses (or high doses) of greater than 700 mg to about 1400 mg in a second step, wherein each second dose (high dose) is administered once every four weeks.

Some aspects of the present disclosure are related to identifying the stage/progression of AD in a subject based on i) the global or overall tau burden in the brain of a human subject or ii) the spread of tau in the subject's brain or portions thereof. In some aspects, the anti-N3pGlu Aβ antibodies of the present disclosure can be administered to the subject i) without determining the stage/progression of AD in the subject or ii) irrespective of the stage/progression of AD in the subject.

In some embodiments, the patients can be stratified/identified/selected/treated based on the amount of tau present in the subject's brain (e.g., in the whole brain or in portions of the brain). In some embodiments, the patients can be stratified/identified/selected/treated based on the amount of tau present in the subject's brain (e.g., in the whole brain or in portions of the brain) and the presence of one or two alleles of APOE4.

In other embodiments, the patients are stratified/identified/selected/treated based on stages of AD progression (e.g., based on the spread of tau in the brain). For example, during some stages, tau burden in an AD patient is isolated to frontal lobe or regions of the temporal lobe that do not include the posterolateral temporal region (PLT). Another stage of AD is where tau burden in an AD patient is limited to the posterolateral temporal (PLT) or occipital regions. Yet another stage of AD is when the tau burden in an AD patient is present in the parietal or precuneus region or in the frontal region along with tau burden in PLT or occipital regions. In some embodiments, the patients can be stratified/identified/selected/treated based on the stages of AD progression (e.g., based on the spread of tau in the brain) and the presence of one or two alleles of APOE4.

The stratification of patients based on amount of tau in the brain, AD progression in portions of brain, and/or the presence of one or two alleles of APOE4 can be used to determine, e.g., whether a patient will respond to anti-N3pGlu Aβ antibody treatments. Stratification/selection of patient population based on amount of tau in the brain, AD progression in portions of brain, and/or the presence of one or two alleles of APOE4 is also helpful in solving the patient heterogeneity and replicability problems faced during design and performance of clinical trials.

Other aspects of the present disclosure provide for human subjects that are responsive to treatment or prevention of a disease characterized by amyloid beta plaques in the brain of a human subject. In some embodiments of this aspect of the present disclosure, the responsive human subjects include human subjects having low to moderate tau burden, very low to moderate tau burden, and/or one or two alleles of APOE4. In some embodiments of this aspect of the present disclosure, the responsive human subjects exclude human subjects with high tau burden. In some embodiments of this aspect of the present disclosure, the responsive human subjects exclude human subjects with high tau burden and/or with one or two alleles of APOE4.

In some embodiments, the anti-N3pGlu Aβ antibody of the present disclosure is administered to the responsive human subjects for treatment or prevention of a disease characterized by amyloid beta plaques in the brain of a human subject. In some embodiments, the anti-N3pGlu Aβ antibody of the present disclosure is administered to the human subjects for treatment or prevention of a disease characterized by amyloid beta plaques in the brain of a human subject. In some embodiments, the anti-N3pGlu Aβ antibody of the present disclosure is administered to the human subjects, irrespective of their brain tau level, for treatment or prevention of a disease characterized by amyloid beta plaques in the brain of the human subject.

In one aspect, the present disclosure is related to a method of treating or preventing a disease characterized by Aβ plaques in the brain of a human subject comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2. In some embodiments of this aspect of the invention, the human subjects are administered the anti-N3pGlu Aβ antibody irrespective of their brain tau levels. Some aspects of the present disclosure are related to a method of treating or preventing a disease characterized by Aβ plaques in the brain of a human subject comprising administering to the subject an anti-N3pGlu Aβ antibody means to reduce Aβ plaques in the brain.

In some embodiments, such treatment results in decrease or reduction in the amyloid deposits, amyloid beta plaques, or Aβ load in brain of the patient having a disease characterized by Aβ plaques. In some embodiments, such treatment results in decrease or reduction in the tau levels in brain of the patient having a disease characterized by Aβ plaques. In some embodiments, such treatment results in decrease or reduction in plasma tau levels in the patient having a disease characterized by Aβ plaques. In some embodiments, the anti-N3pGlu Aβ antibodies of the present disclosure, slow the accumulation of tau pathophysiology, as measured by brain tau PET and/or blood plasma p-Tau.

In some embodiments, such treatment results in decrease or reduction in Neurofilament light chain (NfL) levels in brain of the patient having a disease characterized by Aβ plaques. In some embodiments, such treatment results in increase in Aβ_{42/40} ratio in plasma or cerebrospinal fluid (CSF) of the patient having a disease characterized by Aβ plaques. In some embodiments, such treatment results in decrease or reduction in glial fibrillary acidic protein (GFAP) in blood of the patient having a disease characterized by Aβ plaques. In some embodiments, such treatment results in decrease or reduction in P-tau 217 levels in a patient having a disease characterized by Aβ plaques.

Another aspect of the present disclosure is related to an anti-N3pGlu Aβ antibody for use in the treatment or prevention of a disease characterized by Aβ plaques in the brain of a human subject, wherein the anti-N3pGlu Aβ antibody is for administration of one or more first doses of about 100 mg to about 700 mg, wherein each first dose is administered once about every 4 weeks followed by administration of one or more second doses of greater than 700 mg to about 1400 mg four weeks after administering the one or more first doses, wherein each second dose is administered once about every 4 weeks, and wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

An aspect of the present disclosure is related to a method to reduce amyloid beta plaques in the brain of a human Alzheimer's Disease (AD) subject comprising administering to the subject three first doses of 700 mg of an anti-N3pG Aβ antibody, wherein each first dose is administered at a frequency of once every four weeks; and four weeks after administration of the three first doses, administering to the subject one or more second doses of 1400 mg of the anti-N3pG Aβ antibody at a frequency of once every four weeks; wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

Another aspect of the present disclosure is related to an anti-N3pGlu Aβ antibody for use in the treatment or prevention of a disease characterized by Aβ plaques in the brain of a human subject, wherein one or more first doses of about 100 mg to about 700 mg of the antibody are administered and each first dose is administered once about every 4 weeks followed by administration of one or more second doses of greater than 700 mg to about 1400 mg four weeks after administering the one or more first doses and each second dose is administered once about every 4 weeks, and wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

Another aspect of the present disclosure is related to the use of an anti-N3pGlu Aβ antibody in the manufacture of a medicament for treatment or prevention of a disease characterized by Aβ plaques in the brain of a human subject, wherein one or more first doses of about 100 mg to about 700 mg of the antibody are administered, wherein each first dose is administered once about every 4 weeks followed by administration of one or more second doses of greater than 700 mg to about 1400 mg four weeks after administering the one or more first doses, wherein each second dose is administered once about every 4 weeks, and wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

Another aspect of the present disclosure is related to a method of treating or preventing clinical or pre-clinical Alzheimer's disease, Down's syndrome, or clinical or pre-clinical CAA in a subject, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing preclinical AD (cognitively unimpaired subjects with evidence of AD pathology), prodromal AD (sometimes also referred to as AP-related mild cognitive impairment, MCI or MCI due to AD), mild AD, moderate AD and severe AD, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of slowing cognitive or functional decline in a patient having a disease characterized by Aβ plaques, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of reducing Aβ plaques or Aβ load in a patient having a disease characterized by Aβ plaques, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of slowing functional decline in a patient having a disease characterized by Aβ plaques, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of preventing memory loss, cognitive decline, or functional decline in a patient having a disease characterized by Aβ plaques, comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of slowing disease progression in a human Alzheimer's Disease subject, comprising administering to the subject an anti-N3pGlu Aβ antibody to slow disease progression by at least 15% as measured by Integrated Alzheimer's Disease Rating Scale (iADRS), the administering comprising i) administering to the subject three first doses of 700 mg of the anti-N3pGlu Aβ antibody, wherein each first dose is administered at a frequency of once every four weeks; and ii) four weeks after administration of the three first doses, administering one or more second doses of 1400 mg of the anti-N3pGlu Aβ antibody at a frequency of once every four weeks; and wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

Another aspect of the present disclosure is related to a method of slowing disease progression in a human Alzheimer's Disease subject, comprising administering to the subject an anti-N3pGlu Aβ antibody to slow disease progression by at least 20% as measured by Clinical Dementia Rating Scale—Sum of Boxes (CDR-SB), the administering comprising: i) administering to the subject three first doses of 700 mg of the anti-N3pGlu Aβ antibody, wherein each first dose is administered at a frequency of once every four weeks; and ii) four weeks after administration of the three first doses, administering one or more second doses of 1400 mg of the anti-N3pGlu Aβ antibody at a frequency of once every four weeks; and wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

Another aspect of the present disclosure is related a method of i) reducing or preventing accumulation of brain amyloid beta, ii) reducing or preventing tau accumulation, iii) preventing or delaying onset of memory loss, iv) preventing or delaying cognitive decline, v) preventing or delaying functional decline, or vi) preventing or delaying onset of symptomatic stages of AD in clinically asymptomatic subjects or cognitively unimpaired subjects with evidence of AD pathology. The method includes: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2. In some embodiments, three first doses of about 100 mg to about 700 mg of the anti-N3pGlu Aβ antibody are administered to the patient at a frequency of once every four weeks and about four weeks after administering the one or more first doses, six doses of greater than 700 mg to about 1400 mg of the anti-N3pGlu Aβ antibody at a frequency of once every four weeks are administered to the patient. In some embodiments, three first doses of about 700 mg of the anti-N3pGlu Aβ antibody are administered to the patient at a frequency of once every four weeks and about four weeks after administering the one or more first doses, six doses of about 1400 mg of the anti-N3pGlu Aβ antibody at a frequency of once every four weeks are administered to the patient. In some embodiments, the subject is cognitively unimpaired with evidence of AD pathology. In some embodiments, the subject is clinically asymptomatic with evidence of AD pathology.

Some aspects of the present disclosure are related to a method of treating or preventing a disease characterized by Aβ plaques in the brain of a human subject wherein the human subject is clinically asymptomatic. This method includes: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2. In some embodiments, three first doses of about 100 mg to about 700 mg of the anti-N3pGlu Aβ antibody are administered to the patient at a frequency of once every four weeks and about four weeks after administering the one or more first doses, six doses of greater than 700 mg to about 1400 mg of the anti-N3pGlu Aβ antibody at a frequency of once every four weeks are administered to the patient. In some embodiments, three first doses of about 700 mg of the anti-N3pGlu Aβ antibody are administered to the patient at a frequency of once every four weeks and about four weeks after administering the one or more first doses, six doses of about 1400 mg of the anti-N3pGlu Aβ antibody at a frequency of once every four weeks are administered to the patient.

In some embodiments, the clinically asymptomatic subjects are known to have an Alzheimer's disease-causing genetic mutation. In the present disclosure, “clinically asymptomatic subjects known to have an Alzheimer's disease-causing genetic mutation” include patients known to have a PSEN1 E280A Alzheimer's disease-causing genetic mutation (Paisa mutation), a genetic mutation that causes autosomal-dominant Alzheimer's disease or are at higher risk for developing AD by virtue of carrying one or two APOE4 alleles.

Another aspect of the present disclosure is related to a method of treating or preventing a disease characterized by amyloid beta plaques in the brain of a human subject who has been determined to have very low to moderate tau burden or low to moderate tau burden comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have one or two alleles of APOE4 and very low to moderate tau burden or low to moderate tau burden comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject comprising: determining whether the human subject has very low to moderate tau burden or low to moderate tau burden; and if the human subject has very low to moderate tau burden or low to moderate tau burden, then i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject comprising: determining whether the human subject has very low to moderate tau burden or low to moderate tau burden and one or two alleles of APOE4; and if the human subject has very low to moderate tau burden or low to moderate tau burden and one or two alleles of APOE4, then i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the invention is related to a method of treating or preventing a disease characterized by amyloid beta plaques in the brain of a human subject who has been determined as not having high tau burden comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the invention is related to a method of treating or preventing a disease characterized by amyloid beta plaques in the brain of a human subject who has been determined as not having high tau burden and one or two alleles of APOE4 comprising: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the invention is related to a method of treating or preventing a disease characterized by amyloid beta plaques in the brain of a human subject comprising: determining whether the human subject has high tau burden; and if the human subject does not have high tau burden, then: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the invention is related to a method of treating or preventing a disease characterized by amyloid beta plaques in the brain of a human subject comprising: determining whether the human subject has high tau burden and one or two alleles of APOE4; and if the human subject has one or two alleles of APOE4 and does not have high tau burden, then: i) administering to the human subject one or more first doses of about 100 mg to about 700 mg of an anti-N3pGlu Aβ antibody, wherein each first dose is administered once about every 4 weeks and ii) about four weeks after administering the one or more first doses, administering to the human subject one or more second doses of greater than 700 mg to about 1400 mg of the anti-N3pGlu Aβ antibody, wherein each second dose is administered once about every 4 weeks, wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject comprising administering to the human subject an effective amount of an anti-N3pGlu Aβ antibody, wherein the human subject has been determined as having a very low to moderate tau burden or low to moderate tau burden.

Another aspect of the present disclosure is related to a method of treating or preventing a disease characterized by amyloid beta plaques in the brain of a human subject comprising administering to the human subject an effective amount of an anti-N3pGlu Aβ antibody, wherein the human subject has been determined as having a very low to moderate tau burden or low to moderate tau burden and one or two alleles of APOE4.

Another aspect of the present disclosure is related to a method of treating or preventing a disease characterized by amyloid beta plaques in the brain of a human subject comprising determining whether the human subject has low to moderate tau burden or a very low to moderate tau burden; and if the human subject has low to moderate tau burden or a very low to moderate tau burden, then: administering to the human subject an effective amount of an anti-N3pGlu Aβ antibody.

Another aspect of the present disclosure is related to a method of treating or preventing a disease characterized by amyloid beta plaques in the brain of a human subject comprising determining whether the human subject has one or two alleles of APOE4 and low to moderate tau burden or a very low to moderate tau burden; and if the human subject has one or two alleles of APOE4 and low to moderate tau burden or a very low to moderate tau burden, then: administering to the human subject an effective amount of an anti-N3pGlu Aβ antibody.

Another aspect of the present disclosure is related to a method of treating or preventing a disease characterized by amyloid beta plaques in the brain of a human subject comprising administering to the human subject an effective amount of an anti-N3pGlu Aβ antibody, wherein the human subject has been determined as not having a high tau burden.

Another aspect of the present disclosure is related to a method of treating or preventing a disease characterized by amyloid beta plaques in the brain of a human subject comprising administering to the human subject an effective amount of an anti-N3pGlu Aβ antibody, wherein the human subject has been determined as having one or two alleles of APOE4 and not having a high tau burden.

Another aspect of the present disclosure is related to a method of treating or preventing a disease characterized by amyloid beta plaques in the brain of a human subject comprising determining whether the human subject has high tau burden; and if the human subject does not have high tau burden, then: administering to the human subject an effective amount of an anti-N3pGlu Aβ antibody.

Another aspect of the present disclosure is related to a method of treating or preventing a disease characterized by amyloid beta plaques in the brain of a human subject comprising determining whether the human subject has one or two alleles of APOE4 and a high tau burden; and if the human subject has one or two alleles of APOE4 and does not have high tau burden, then: administering to the human subject an effective amount of an anti-N3pGlu Aβ antibody.

In some aspects, an anti-N3pGlu Aβ antibody may be used to decrease, prevent further increase of tau burden, or slow the rate of tau accumulation in different portions of a human brain, e.g., in different lobes of the brain of a human subject. In some embodiments, the anti-N3pGlu Aβ antibodies is used to decrease, prevent further increase, or slow the rate of tau burden/accumulation in the frontal lobe of the human brain. In some embodiments, tau accumulation in the frontal lobe is slowed by at least 30-70% as compared to untreated subjects. In some embodiments, tau accumulation in the frontal lobe is slowed by at least 50% as compared to untreated subjects. In some embodiments, the subject has a negative tau PET imaging scan in a frontal lobe brain region prior to administering the anti-N3pGlu Aβ antibody. In some embodiments, the subject has a brain tau level of less than 0.4 SUVr in a frontal lobe region 76 weeks after the administration of the anti-N3pGlu Aβ antibody, wherein the brain tau level is measured by tau PET imaging scan.

In some embodiments, the anti-N3pGlu Aβ antibodies is used to decrease, prevent further increase, or slow the rate of tau burden/accumulation in the parietal lobe of the human brain. In some embodiments, the subject has an increase in tau level in a parietal lobe of less than 0.06 SUVr 76 weeks after the administration of the anti-N3pGlu Aβ antibody, wherein the brain tau level is measured by tau PET imaging scan.

In some embodiments, the anti-N3pGlu Aβ antibodies is used to decrease, prevent further increase, or slow the rate of tau burden/accumulation in the occipital lobe of the human brain. In some embodiments, the anti-N3pGlu Aβ antibodies is used to decrease, prevent further increase, or slow the rate of tau burden/accumulation in the temporal lobe of the human brain. In some embodiments, the anti-N3pGlu Aβ antibodies is used to decrease, prevent further increase, or slow the rate of tau burden/accumulation in the posterolateral temporal lobe. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

An aspect of the present disclosure is related to a method of treating or preventing a disease characterized by amyloid beta plaques in the brain of a human subject who has been determined to have tau burden in the temporal lobe of the brain and/or one or two alleles of APOE4 wherein the method comprises administering an anti-N3pGlu Aβ to the human subject. Another aspect of the invention is related to a method of treating or preventing a disease characterized by amyloid beta plaques in the brain of a human subject comprising determining whether the human subject has tau burden in the temporal lobe of the brain and/or one or two alleles of APOE4 and administering an anti-N3pGlu Aβ to the human subject. In some embodiments, the human subject has tau burden in the posterolateral temporal lobe and/or one or two alleles of APOE4. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden in the occipital lobe of the brain and/or one or two alleles of APOE4 wherein the method comprises administering an anti-N3pGlu Aβ to the human subject. Another aspect of the invention is related to a method of treating or preventing a disease characterized by amyloid beta plaques in the brain of a human subject comprising determining whether the human subject has tau burden in the occipital lobe of the brain and/or one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden in the parietal lobe of the brain and/or one or two alleles of APOE4 wherein the method comprises administering an anti-N3pGlu Aβ to the human subject. Another aspect of the invention is related to a method of treating or preventing a disease characterized by amyloid beta plaques in the brain of a human subject comprising determining whether the human subject has tau burden in the parietal lobe of the brain and/or one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden in the frontal lobe of the brain and/or one or two alleles of APOE4 wherein the method comprises administering an anti-N3pGlu Aβ to the human subject. Another aspect of the invention is related to a method of treating or preventing a disease characterized by amyloid beta plaques in the brain of a human subject comprising determining whether the human subject has tau burden in the frontal lobe of the brain and/or one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden in the posterolateral temporal (PLT) and/or occipital lobe of the brain and/or one or two alleles of APOE4 wherein the method comprises administering an anti-N3pGlu Aβ to the human subject. Another aspect of the invention is related to a method of treating or preventing a disease characterized by amyloid beta plaques in the brain of a human subject comprising determining whether the human subject has tau burden in the posterolateral temporal (PLT) and/or occipital lobe of the brain and/or one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden in i) parietal or precuneus region or ii) in frontal region along with tau burden in PLT or occipital regions of the brain and/or iii) one or two alleles of APOE4 wherein the method comprises administering an anti-N3pGlu Aβ to the human subject. Another aspect of the invention is related to a method of treating or preventing a disease characterized by amyloid beta plaques comprising determining whether the human subject has tau burden in i) parietal or precuneus region or ii) in the frontal region along with tau burden in PLT or occipital regions of the brain and/or iii) one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

Another aspect of the present disclosure is related to a method of treating or preventing a disease characterized by amyloid beta plaques in the brain of a human subject who has been determined to have tau burden i) isolated to frontal lobe or ii) in regions of the temporal lobe that do not include the posterolateral temporal region (PLT) of the brain and/or iii) one or two alleles of APOE4 wherein the method comprises administering an anti-N3pGlu Aβ to the human subject. Another aspect of the invention is related to a method of treating or preventing a disease characterized by amyloid beta plaques comprising determining whether the human subject has tau burden i) isolated to frontal lobe or ii) in regions of the temporal lobe that do not include the posterolateral temporal region (PLT) of the brain and/or iii) one or two alleles of APOE4 and administering an anti-N3pGlu Aβ to the human subject. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

In some aspects, the present disclosure is related to a method of selecting a human subject for treatment or prevention of a disease characterized by amyloid beta plaques in the brain of a human subject. In some embodiments, the human subject is selected based on the amount of global (overall) tau in the brain of the human subject. For example, the human subject is selected for treatment or prevention of a disease characterized by amyloid beta plaques in the brain because the patient has very low to moderate tau in the brain and/or one or two alleles of APOE4. In another embodiment, the human subject is selected for treatment or prevention of a disease characterized by amyloid beta plaques in the brain because the patient has low to moderate tau (or intermediate tau) in the brain and/or one or two alleles of APOE4. In another embodiment, the human subject is excluded from treatment or prevention of a disease characterized by amyloid beta plaques in the brain because the patient has high tau in the brain. In some embodiments, the human subject is selected based on progression of AD in the brain of the human subject. For example, the human subject is selected for treatment or prevention of a disease characterized by amyloid beta plaques in the brain because the patient has tau burden present in the frontal lobe of the brain and/or one or two alleles of APOE4. In another embodiment, the human subject is selected for treatment or prevention of a disease characterized by amyloid beta plaques in the brain because the patient has tau burden present in the parietal lobe of the brain and/or one or two alleles of APOE4. In another embodiment, the human subject is selected for treatment or prevention of a disease characterized by amyloid beta plaques in the brain because the patient has tau burden present in the occipital lobe of the brain and/or one or two alleles of APOE4. In another embodiment, the human subject is selected for treatment or prevention of a disease characterized by amyloid beta plaques in the brain because the patient has tau burden present in the temporal lobe of the brain and/or one or two alleles of APOE4. In some embodiments, the human subject is selected for treatment or prevention of a disease characterized by amyloid beta plaques in the brain because the patient has tau burden present in the posterolateral temporal (PLT) and/or occipital lobe of the brain and/or one or two alleles of APOE4. In some embodiments, the human subject is selected for treatment or prevention of a disease characterized by amyloid beta plaques in the brain because the patient has tau burden present in i) parietal or precuneus region, ii) in frontal region along with tau burden in PLT or occipital regions of the brain and/or, iii) one or two alleles of APOE4. In some embodiments, the human subject is selected for treatment or prevention of a disease characterized by amyloid beta plaques in the brain because the patient has tau burden i) isolated to frontal lobe ii) in regions of the temporal lobe that do not include the posterolateral temporal region (PLT) of the brain and/or iii) one or two alleles of APOE4. In some embodiments, the human subject is administered i) one or more first doses of about 100 mg to about 700 mg of the anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

In some embodiments, the subject described in the various aspects of the present disclosure has been determined to have a posterior-lateral temporal lobe tau burden and/or one or two alleles of APOE4. In some embodiments, the subject described in the various aspects of the present disclosure has been determined to have posterior-lateral temporal lobe and occipital lobe tau burden and/or one or two alleles of APOE4. In some embodiments, the subject described in the various aspects of the present disclosure has been determined to have posterior-lateral temporal lobe, occipital lobe, and parietal lobe tau burden and/or one or two alleles of APOE4. In some embodiments, the subject described in the various aspects of the present disclosure has been determined to have posterior-lateral temporal lobe, occipital lobe, parietal lobe, and frontal lobe tau burden and/or one or two alleles of APOE4. In some embodiments, the subject described in the various aspects of the present disclosure has been determined to have posterior-lateral temporal lobe, occipital lobe, parietal lobe and/or frontal lobe tau burden and/or one or two alleles of APOE4. In some embodiments, the subject described in the various aspects of the present disclosure has been determined to have posterior-lateral temporal lobe, occipital lobe, parietal lobe and/or frontal lobe tau burden corresponds a neurological tau burden of greater than 1.46 SUVr based on PET imaging. In some embodiments, the anti-N3pGlu Aβ antibody of the present disclosure limits an increase in the subject's frontal lobe tau over 72 weeks to less than 0.04 SUVr as measured by tau PET imaging.

In some embodiments, tau burden in the human brain or a portion thereof (e.g., in a lobe of the brain or whole brain) can be used to determine whether administration of the anti-N3pGlu Aβ antibody should be discontinued. For instance, slowing in rate of removal of tau, a stop in reduction of tau levels, prevention of further increase in tau levels, or slowing of the rate of tau accumulation in the brain can be used as metric to determine the duration of administration of the anti-N3pGlu Aβ antibody. In some embodiments, anti-N3pGlu Aβ antibody is administered to the subject until there is a slowing in the rate of removal of tau, a stop in reduction of tau levels, prevention of further increase in tau levels, slowing of the rate of tau accumulation in the brain, or slowing in the rate of tau accumulation in the temporal lobe, the occipital lobe, the parietal lobe, or the frontal lobe.

In some embodiments, amyloid beta burden in the human brain can be used to determine whether administration of the anti-N3pGlu Aβ antibody should be discontinued. For instance, slowing in rate of removal of Aβ, a stop in reduction of Aβ levels, prevention of further increase in Aβ levels, or slowing of the rate of Aβ accumulation in the brain can be used as metric to determine the duration of administration of the anti-N3pGlu Aβ antibody. In some embodiments, administration of the anti-N3pGlu Aβ antibody of the present disclosure is stopped if Aβ plaques in the brain of the subject reach normal levels by 24 weeks or Aβ plaques level in the brain of the subject stop reducing. In some embodiments, the level of Aβ plaques in the brain of the subject is sustained at normal levels for at least 52 weeks after the administration of the anti-N3pGlu Aβ antibody is stopped. In some embodiments, administering the anti-N3pGlu Aβ antibody reduces the level of Aβ plaques in the brain of the subject to normal levels by 24 weeks. In some embodiments, the level of Aβ plaques in the brain of the subject is sustained at normal levels for at least 52 additional weeks.

In some embodiments, the tau burden present in a portion of the brain of a human subject can be used for selection of optimal treatment regimens or for administration of therapeutic modalities in combination with an anti-N3pGlu Aβ antibody. For example, the presence of tau burden in the frontal lobe of the brain of an amyloid positive human subject can be used as a metric to determine whether the human subject will benefit from administration of an anti-N3pGlu Aβ antibody alone or its combination with an anti-tau antibody. In some embodiments, an anti-N3pGlu Aβ antibody in combination with an anti-tau antibody may be used to decrease, prevent further increase, or slow the rate of tau accumulation in different portions of a human brain, e.g., in different lobes of the brain of a human subject. In some embodiments, the tau burden in different portions of a human brain, e.g., in different lobes of the brain of a human subject can be used for i) tracking patient's response to treatment or ii) when a therapy may need to be reinitiated. In some embodiments, the antibodies, methods, or dosing regimens described in various aspects of the present disclosure cause: i) reduction in Aβ plaques in the brain of the human subject and/or ii) slows cognitive decline or functional decline in the human subject. In some embodiments, the antibodies, methods, or dosing regimens described herein this method results in reduction of amyloid plaques.

The anti-N3pGlu Aβ antibodies described in various aspects of the present disclosure i) include, ii) may be replaced with, or iii) used along with anti-N3pGlu Aβ antibodies such as:
an anti-N3pGlu Aβ antibody comprising: light chain complementarity determining region 1 (LCDR1) having an amino acid sequence of SEQ ID NO: 5, light chain complementarity determining region 2 (LCDR2) having an amino acid sequence of SEQ ID NO: 6, and light chain complementarity determining region 3 (LCDR3) having an amino acid sequence of SEQ ID NO: 7 or an amino acid sequence having at least 95% homology to light chain complementarity determining region 1 (LCDR1) of SEQ ID NO: 5, an amino acid sequence having at least 95% homology to light chain complementarity determining region 2 (LCDR2) of SEQ ID NO: 6, and an amino acid sequence having at least 95% homology to light chain complementarity determining region 3 (LCDR3) of SEQ ID NO: 7;
an anti-N3pGlu Aβ antibody comprising: heavy chain complementarity determining region 1 (HCDR1) having an amino acid sequence of SEQ ID NO: 8, heavy chain complementarity determining region 2 (HCDR2) having an amino acid sequence of SEQ ID NO: 9, and heavy chain complementarity determining region 3 (HCDR3) having an amino acid sequence of SEQ ID NO: 10 or an amino acid sequence having at least 95% homology to heavy chain complementarity determining region 1 (HCDR1) of SEQ ID NO: 8, an amino acid sequence having at least 95% homology to heavy chain complementarity determining region 2 (HCDR2) of SEQ ID NO: 9, and an amino acid sequence having at least 95% homology to heavy chain complementarity determining region 3 (HCDR3) of SEQ ID NO: 10;
an anti-N3pGlu Aβ antibody comprising: light chain complementarity determining region 1 (LCDR1) having an amino acid sequence of SEQ ID NO: 5, light chain complementarity determining region 2 (LCDR2) having an amino acid sequence of SEQ ID NO: 6, light chain complementarity determining region 3 (LCDR3) having an amino acid sequence of SEQ ID NO: 7, heavy chain complementarity determining region 1 (HCDR1) having an amino acid sequence of SEQ ID NO: 8, heavy chain complementarity determining region 2 (HCDR2) having an amino acid sequence of SEQ ID NO: 9, and heavy chain complementarity determining region 3 (HCDR3) having an amino acid sequence of SEQ ID NO: 10 or amino acid sequence having at least 95% homology to light chain complementarity determining region 1 (LCDR1) of SEQ ID NO: 5, amino acid sequence having at least 95% homology to light chain complementarity determining region 2 (LCDR2) of SEQ ID NO: 6, amino acid sequence having at least 95% homology to light chain complementarity determining region 3 (LCDR3) of SEQ ID NO: 7, amino acid sequence having at least 95% homology to heavy chain complementarity determining region 1 (HCDR1) of SEQ ID NO: 8, amino acid sequence having at least 95% homology to heavy chain complementarity determining region 2 (HCDR2) of SEQ ID NO: 9, and amino acid sequence having at least 95% homology to heavy chain complementarity determining region 3 (HCDR3) of SEQ ID NO: 10;
an anti-N3pGlu Aβ antibody comprising: a LCVR and a HCVR, wherein said LCVR comprises: LCDR1, LCDR2 and LCDR3 and HCVR comprises HCDR1, HCDR2 and HCDR3, which are selected from the group consisting of LCDR1 is SEQ ID NO: 5, LCDR2 is SEQ ID NO: 6, LCDR3 is SEQ ID NO: 7, HCDR1 is SEQ ID NO: 8, HCDR2 is SEQ ID NO: 9, and HCDR3 is SEQ ID NO: 10 or a LCVR and a HCVR, wherein said LCVR comprises LCDR1, LCDR2 and LCDR3 and HCVR comprises HCDR1, HCDR2 and HCDR3, which are selected from the group consisting of LCDR1 having at least 95% homology to SEQ ID NO: 5, LCDR2 having at least 95% homology to SEQ ID NO: 6, LCDR3 having at least 95% homology to SEQ ID NO: 7, HCDR1 having at least 95% homology to SEQ ID NO: 8, HCDR2 having at least 95% homology to SEQ ID NO: 9, and HCDR3 having at least 95% homology to SEQ ID NO: 10.
an N3pGlu Aβ antibody comprising a light chain (LC) comprising: the amino acid sequence of SEQ ID NO: 3 or amino acid sequence having at least 95% homology to SEQ ID NO: 3;
an N3pGlu Aβ antibody comprising a heavy chain (HC) comprising: the amino acid sequence of SEQ ID NO: 4 or amino acid sequence having at least 95% homology to SEQ ID NO: 4;
an anti-N3pGlu Aβ antibody comprising a LC and a HC, wherein the LC comprises the amino acid sequence of SEQ ID NO: 3 and the HC comprises the amino acid sequence of SEQ ID NO: 4 or wherein the LC comprises amino acid sequence having at least 95% homology to SEQ ID NO: 3 and the HC comprises amino acid sequence having at least 95% homology to SEQ ID NO: 4;
an anti-N3pGlu Aβ antibody comprising two light chains and two heavy chains, wherein the LC comprises amino acid sequence of SEQ ID NO: 3 or amino acid sequence having at least 95% homology to SEQ ID NO: 3 and the HC comprises the amino acid sequence of SEQ ID NO: 4 or amino acid sequence having at least 95% homology to SEQ ID NO: 4.
an N3pGlu Aβ antibody comprising a LCVR comprising the amino acid sequence of SEQ ID NO: 1 or amino acid sequence having at least 95% homology to SEQ ID NO: 1;
an N3pGlu Aβ antibody comprising a HCVR comprising the amino acid sequence of SEQ ID NO: 2 or amino acid sequence having at least 95% homology to SEQ ID NO: 2.
an N3pGlu Aβ antibody comprising a LCVR and a HCVR wherein the LCVR comprises the amino acid sequence of SEQ ID NO: 1 or amino acid sequence having at least 95% homology to SEQ ID NO: 1; and the HCVR comprises the amino acid sequence of SEQ ID NO: 2 or amino acid sequence having at least 95% homology to SEQ ID NO: 2.

In some embodiments, the anti-N3pGlu Aβ antibodies of the present disclosure include kappa LC and IgG HC. In a particular embodiment, the anti-N3pGlu Aβ antibodies of the present disclosure are of the human IgG1 isotype.

In some embodiments, the human subject is administered one or more first doses of about 100 mg to about 700 mg of the anti-N3pGlu Aβ antibody as described herein. In some embodiments, the one or more first doses are administered to the human subject such that each first dose is administered once every four weeks. In particular embodiments, the first dose is administered to the subject once. In some embodiments, the first dose is administered to the subject twice wherein each first dose is administered once every four weeks. In some embodiments, the first dose is administered to the subject three times wherein each first dose is administered once every four weeks.

In some embodiments, the subject is administered one first dose, two first doses, or three first doses of about 100 mg to about 700 mg, wherein each first dose is administered once about every four weeks. In a particular embodiment, the human subject is administered three first doses of about 700 mg wherein each first dose is administered once about every four weeks. In some embodiments, the human subject is administered the first dose once, two times, or three times before administering the second dose.

In some embodiments, three first doses of about 700 mg are administered to the subject once every 4 weeks for a duration of 12 weeks followed by second doses of about 1400 mg. In some embodiments, the one or more first doses of about 700 mg are administered to the subject once every 4 weeks over a duration of about 3 months followed by second doses of about 1400 mg.

In some embodiments, the first dose is about 100 mg, about 200 mg, about 300 mg, about 400 mg, about 500 mg, about 600 mg, about 700 mg. In some embodiments, the first dose from about 1 mg/kg to about 10 mg/kg of the anti-N3pGlu Aβ antibody. In particular embodiments, the subject is administered up to three first doses of about 1 mg/kg to about 10 mg/kg. In some embodiments, the subject is administered one first dose, two first doses, or three first doses of about 1 mg/kg to about 10 mg/kg. In one particular embodiment, the subject is administered three first doses of about 10 mg/kg once every four weeks. In some embodiments, the first dose is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.

In a particular embodiment, the first dose is administered once every 4 weeks or once every month. In one particular embodiment, the subject is administered three first doses of about 10 mg/kg once every 4 weeks. In some embodiments, the first dose of the anti-N3pGlu Aβ antibody is administered to the subject for about one month, about two months, or about three months.

In some embodiments, the subject is administered one or more second doses of greater than 700 mg to about 1400 mg of the anti-N3pGlu Aβ antibody. In some embodiments, the subject is administered one or more second doses of greater than 700 mg to about 1400 mg of the anti-N3pGlu Aβ antibody wherein each second dose is administered once about every 4 weeks. In some embodiments, the second dose is administered 4 weeks after the one or more first doses.

In particular embodiments, the subject is administered one or more second doses of greater than 700 mg. In some embodiments, the subject is administered one or more second doses of about 1400 mg. In some embodiments, the second dose is greater than 700 mg, about 800 mg, about 900 mg, about 1000 mg, about 1100 mg, about 1200 mg, about 1300 mg or about 1400. In a particular embodiment, the second dose is administered once every 4 weeks. In one particular embodiment, the subject is administered one or more second doses of greater than 700 mg once every 4 weeks. In one particular embodiment, the subject is administered one or more second doses of about 1400 mg once every 4 weeks.

A brain MRI scan may be administered to the human subject to monitor/evaluate a human subject (e.g., for ARIA-E or ARIA-H). In some embodiments, a brain MRI scan can be administered to the human subject to diagnose/evaluate/monitor adverse event(s) caused by administration of anti-N3pGlu Aβ antibody. In some embodiments, the human subject is administered a brain MRI scan in between administration of doses of the anti-N3pGlu Aβ antibody (e.g., once every 4 weeks). In some embodiments, a baseline brain MRI is obtained prior to initiating treatment with the anti-N3pGlu Aβ antibodies. In some embodiments, the human subject is administered a brain MRI scan before increasing the dose of the anti-N3pGlu Aβ antibody, e.g., from 700 mg to 1400 mg. In some embodiments, the human subject is administered a brain MRI scan after the first dose of the anti-N3pGlu Aβ antibody. In some embodiments, the human subject is administered a brain MRI scan after three doses of the anti-N3pGlu Aβ antibody. In some embodiments, the human subject is administered a brain MRI scan after the first four weeks of initiation of treatment. In some embodiments, the human subject is administered a brain MRI scan after the first 12 weeks of initiation of treatment. In some embodiments, the human subject is administered a brain MRI scan before administering a 1400 mg dose. In some embodiments, a brain MRI scan is performed before starting administration of the one or more second doses of 1400 mg. In some embodiments, the human subject is administered a brain MRI scan before administering a 20 mg/kg dose. In some embodiments, the human subject is administered a brain MRI scan after the last dose 700 mg dose. In some embodiments, the human subject is administered a brain MRI scan after the last dose 10 mg/kg dose. In some embodiments, the methods of the present disclosure include a step of evaluating MRI scan of the subject's brain for amyloid-related imaging abnormality (ARIA) after the administration of the three first doses and prior to the administration of the one or more second doses.

In some embodiments, the present disclosure is related to a method of treating Alzheimer's Disease in a subject in need thereof comprising: i) administering to the subject three first doses of 700 mg of the anti-N3pGlu Aβ antibody, wherein each first dose is administered at a frequency of once every four weeks; ii) evaluating magnetic resonance image (MRI) scan of the subject's brain for amyloid-related imaging abnormality (ARIA), after the administration of the three first doses and prior to the administration of the one or more second doses wherein the administration of one or more second doses is temporarily withheld if symptoms consistent with ARIA occur; iii) four weeks after administration of the three first doses, administering one or more second doses of 1400 mg of the anti-N3pGlu Aβ antibody at a frequency of once every four weeks; and wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2. In some embodiments, the administration of one or more second doses is re-initiated after resolution of ARIA symptoms or radiographic stabilization on MRI. In some embodiments, the one or more second doses are withheld, and corticosteroids are administered to the subject.

In some embodiments, the present disclosure is related to a method of treating Alzheimer's Disease in a subject in need thereof comprising: i) administering to the subject three first doses of 700 mg of the anti-N3pGlu Aβ antibody, wherein each first dose is administered at a frequency of once every four weeks; ii) evaluating magnetic resonance image (MRI) scan of the subject's brain for amyloid-related imaging abnormality (ARIA), after the administration of the three first doses and prior to the administration of the one or more second doses wherein the administration of one or more second doses is discontinued if symptoms consistent with severe or symptomatic ARIA occur; iii) four weeks after administration of the three first doses, administering one or more second doses of 1400 mg of the anti-N3pGlu Aβ antibody at a frequency of once every four weeks; and wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2. In some embodiments, the administration of one or more second doses is discontinued, and corticosteroids are administered to the subject.

In some embodiments, the present disclosure is related to a method of treating Alzheimer's Disease in a subject in need thereof until symptoms consistent with ARIA-E occur comprising: i) administering to the subject three first doses of 700 mg of the anti-N3pGlu Aβ antibody, wherein each first dose is administered at a frequency of once every four weeks; and ii) four weeks after administration of the three first doses, administering one or more second doses of 1400 mg of the anti-N3pGlu Aβ antibody at a frequency of once every four weeks; wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2. In some embodiments, the symptoms of ARIA are detected by MRI or are presented in the subject.

In some embodiments, the present disclosure is related to a method for treating a patient with donanemab, wherein the patient is suffering from Alzheimer's disease, the method comprising the steps of: a) administering [or having administered] 700 mg of donanemab every four weeks for the first three doses; b) determining whether the patient has symptoms of ARIA-E i) by performing or having performed an MRI prior to dose increase or ii) if clinical symptoms consistent with ARIA-E occur; and c) if the patient has moderate symptoms of ARIA-E, temporarily discontinuing treatment with donanemab; and d) if the patient does not have symptomatic ARIA-E, administering donanemab to the patient in an amount of 1400 mg every four weeks until brain amyloid is cleared, is negative, or is <24.1 CL.

In some embodiments, the present disclosure is related to a method for treating a patient with donanemab, wherein the patient is suffering from Alzheimer's disease, the method comprising the steps of: a) administering [or having administered] 700 mg of donanemab every four weeks for the first three doses; b) determining whether the patient has symptoms of ARIA-E i) by performing or having performed an MRI prior to dose increase or ii) if clinical symptoms consistent with ARIA-E occur; and if the patient does not have symptomatic ARIA-E, administering donanemab to the patient in an amount of 1400 mg every four weeks until brain amyloid is cleared, is negative, or is <24.1 CL.

In some embodiments, the present disclosure is related to an improved method for treating a patient with donanemab to a patient suffering from Alzheimer's disease, wherein the improvement comprises: a) administering or having administered 700 mg of donanemab every four weeks for the first three doses; b) determining whether the patient has symptoms of ARIA-E i) by performing or having performed an MRI prior to dose increase or ii) if clinical symptoms consistent with ARIA-E occur; and c) if the patient has moderate symptoms of ARIA-E, temporarily discontinuing treatment with donanemab; and d) if the patient does not have symptomatic ARIA-E, internally administering donanemab to the patient in an amount of 1400 mg every four weeks until brain amyloid is cleared, is negative, or is <24.1 CL.

In some embodiments, the present disclosure is related to an improved method for treating a patient with donanemab to a patient suffering from Alzheimer's disease, wherein the improvement comprises: a) administering or having administered 700 mg of donanemab every four weeks for the first three doses; b) determining whether the patient has symptoms of ARIA-E i) by performing or having performed an MRI prior to dose increase or ii) if clinical symptoms consistent with ARIA-E occur; and if the patient does not have symptomatic ARIA-E, internally administering donanemab to the patient in an amount of 1400 mg every four weeks until brain amyloid is cleared, is negative, or is <24.1 CL.

In some embodiments, the present disclosure is related to a method for treating a patient with donanemab, wherein the patient is suffering from Alzheimer's Disease, the method comprising the steps of: a) administering or having administered 700 mg of donanemab every four weeks for the first three doses; b) discontinuing treatment if the patient has moderate symptoms of ARIA-E; and c) continuing treatment once ARIA-E resolves by administering donanemab to the patient in an amount of 1400 mg every four weeks until brain amyloid is cleared, is negative, is <24.1 CL, or ARIA-E symptoms reappear. In some embodiments, the symptoms or ARIA-E are confirmed or are determined by an MRI scan.

In some embodiments, the present disclosure is related to a method for treating a patient with donanemab, wherein the patient is suffering from Alzheimer's disease, the method comprising the steps of: a) administering or having administered 700 mg of donanemab every four weeks for the first three doses; b) administering donanemab to the patient in an amount of 1400 mg every four weeks until brain amyloid is cleared, is negative, or is <24.1 CL so long as the patient does not have symptomatic ARIA-E. In some embodiments, the symptoms or ARIA-E are confirmed or are determined by an MRI scan.

In some embodiments, a brain MRI of a patient is obtained prior to dose increase (e.g., from 700 mg to 1400 mg) or if symptoms consistent with ARIA-E occur. In some embodiments, the treatment with anti-N3pGlu Aβ antibodies is withheld or discontinued due to or upon occurrence of severe or symptomatic ARIA-E. In some embodiments, upon occurrence of mild or moderate asymptomatic ARIA-E in patients, treatment with anti-N3pGlu Aβ antibodies may be temporarily interrupted. In some embodiments, upon occurrence of mild or moderate asymptomatic ARIA-E in patients, the dose of anti-N3pGlu Aβ antibodies may be temporarily reduced from 1400 mg to 700 mg. In some embodiments—upon occurrence of ARIA-E—supportive therapy including corticosteroids may be administered to the patient. In some embodiments, treatment with anti-N3pGlu Aβ antibodies may be re-initiated after resolution of symptoms or radiographic stabilization on abnormal brain MRI.

If symptoms of ARIA-H occur, it is often in the presence of ARIA-E and managed accordingly as for ARIA-E. In some embodiments, a brain MRI of a patient is obtained prior to dose increase or if symptoms consistent with ARIA-H occur. In some embodiments, the treatment with anti-N3pGlu Aβ antibodies is withheld or discontinued due to or upon occurrence of ARIA-H. In some embodiments-upon occurrence of ARIA-H in patients-treatment with anti-N3pGlu Aβ antibodies may be temporarily interrupted, e.g., when ARIA-H symptoms are mild or moderate. In some embodiments, upon occurrence of mild or moderate asymptomatic ARIA-H in patients, the dose of anti-N3pGlu Aβ antibodies may be temporarily reduced from 1400 mg to 700 mg. In some embodiments, upon occurrence of ARIA-H supportive therapy including corticosteroids may be administered to the patient. In some embodiments, treatment with anti-N3pGlu Aβ antibodies may be temporarily discontinued until symptoms of ARIA-E or ARIA-H improve.

In some embodiments, the subject is administered one or more second doses of greater than 10 mg/kg to about 20 mg/kg of the anti-N3pGlu Aβ antibody. In some embodiments, the second dose is greater than 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg or about 20 mg/kg. In one embodiment, the subject is administered one or more second doses of greater than 10 mg/kg. In one embodiment, the subject is administered one or more second doses of about 20 mg/kg. In a particular embodiment, the first dose is administered once every month. In one embodiment, the subject is administered one or more second doses of greater than 10 mg/kg, wherein each second dose is administered once every 4 weeks or once every month. In one embodiment, the subject is administered one or more second doses of about 20 mg/kg, wherein each second dose is administered once every 4 weeks or once every month.

In some embodiments, the first dose of the anti-N3pGlu Aβ antibody is administered to the subject once followed by one or more second doses, wherein the second dose is administered 4 weeks after the one or more first doses and once every 4 weeks thereafter. In some embodiments, the first doses of the anti-N3pGlu Aβ antibody are administered to the subject two times (once every four weeks) followed by one or more second doses which are administered after 4 weeks of the first doses and once every 4 weeks thereafter. In some embodiments, the first doses of the anti-N3pGlu Aβ antibody are administered to the subject three times (once every four weeks) followed by one or more second doses which are administered after 4 weeks of the first doses and once every 4 weeks thereafter.

In some embodiments, the subject is treated with one or more first doses, one or more second doses of about 1400 mg, and subsequently with one or more second doses of greater than 700 mg to about 1300 mg. In one particular embodiment, the subject is treated with one or more first doses of about 700 mg, one or more second doses of about 1400 mg, and subsequently with one or more doses of about 700 mg.

In some embodiments, the anti-N3pGlu Aβ antibody slows disease progression in patients with early symptomatic Alzheimer's disease and with the presence of intermediate brain tau burden. In some embodiments, the patient is administered 700 mg of the anti-N3pG Aβ antibody every 4 weeks for the first 3 doses, followed by 1400 mg of the anti-N3pG Aβ antibody every 4 weeks, until brain amyloid plaque reaches a normal range. In some embodiments, an MRI is performed on the patient prior to increasing the dose of the anti-N3pG Aβ antibody from 700 mg to 1400 mg.

In some embodiments, the anti-N3pGlu Aβ antibody slows disease progression in patients with early symptomatic Alzheimer's disease (i.e., patients having mild cognitive impairment or mild dementia due to AD). In some embodiments, the anti-N3pGlu Aβ antibody shows clinical benefit(s) in patients who are amyloid positive and have intermediate brain tau burden. In some embodiments, the patient is administered 700 mg of the anti-N3pGlu Aβ antibody every 4 weeks for the first 3 doses, followed by 1400 mg of the anti-N3pGlu Aβ antibody every 4 weeks, until brain amyloid plaque is cleared. In some embodiments, a brain MRI is performed on the patient prior to increasing the dose of the anti-N3pG Aβ antibody from 700 mg to 1400 mg. In some embodiments, a baseline brain MRI is obtained prior to initiating treatment.

In some embodiments, the anti-N3pGlu Aβ antibody slows disease progression in patients with early symptomatic Alzheimer's disease (mild cognitive impairment or mild dementia due to AD) with biomarker evidence consistent with AD neuropathology. In some embodiments, the patient is administered 700 mg of the anti-N3pGlu Aβ antibody every 4 weeks for the first 3 doses, followed by 1400 mg of the anti-N3pGlu Aβ antibody every 4 weeks, until brain amyloid plaque is cleared. In some embodiments, a brain MRI is performed on the patient prior to increasing the dose of the anti-N3pG Aβ antibody from 700 mg to 1400 mg. In some embodiments, a baseline brain MRI is obtained prior to initiating treatment. In some embodiments, if a dose/infusion of the anti-N3pGlu Aβ antibody is missed, administration of the anti-N3pGlu Aβ antibody is resumed at the same dosing schedule as appropriate.

In some embodiments, the dosing regimen of the present disclosure includes one or more additional doses (also referred to herein as third dose(s)) after the one or more first doses of about 100 mg to about 700 mg and the one or more second doses of greater than 700 mg to about 1400 mg. In some embodiments, the third dose is administered to the subject to reduce the deposition of Aβ in the brain of the subject, prevent further deposition of Aβ in the brain of the subject, prevent further cognitive decline, prevent memory loss, or prevent functional decline. The third dose could be from about 100 mg to about 1400 mg. In some embodiments different or same antibodies are used for the first dose, the second dose, and the third doses. In some embodiments, a different Aβ targeting antibody is administered in the third dose. For instance, some embodiments of the present disclosure include i) administering to the human subject one or more first doses of about 100 mg to about 700 mg of the anti-N3pGlu Aβ antibody, wherein each first dose is administered once about every 4 weeks; ii) about four weeks after administering the one or more first doses, administering to the human subject one or more second doses of greater than 700 mg to about 1400 mg of the anti-N3pGlu Aβ antibody, wherein each second dose is administered once about every 4 weeks, and iii) subsequently administering one or more third doses of about 100 mg to about 1400 mg of the anti-N3pGlu Aβ antibody, wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2. In some embodiments, one or more third doses of the anti-N3pGlu Aβ antibodies of the present disclosure can be administered to the subject every 2 or 4 weeks, every month, every 1 year, every 2 years, every 3 years, every 4 years, every 5 years, or every 10 years. In some embodiments, the third dose is given every 2 weeks. In some embodiments, the third dose is given every 4 weeks. In some embodiments, the third dose is given every year. In an embodiment, the third dose is given every 2 years. In another embodiment, the third dose is given every 3 years. In another embodiment, the third dose of the antibody is given every 5 years. In another embodiment, the third dose of the antibody is given every 10 years. In another embodiment, the third dose of the antibody is given every 2 to 5 years. In another embodiment, the third dose of the antibody is given every 5 to 10 years.

In some embodiments, the anti-N3pGlu Aβ antibody is administered to the subject for a duration sufficient to treat or prevent the disease. In some embodiments, the anti-N3pGlu Aβ antibody (including the first doses of the antibody and the second doses of the antibody) is administered to the subject for a duration of up to about 72 weeks, optionally, once every 4 weeks or once every month. In some embodiments, the anti-N3pGlu Aβ antibody (including the first doses of the antibody and the second doses of the antibody) is administered to the subject for a duration of up to about 98 weeks, optionally, once every 4 weeks or once every month. In some embodiments, the anti-N3pGlu Aβ antibody (including the first doses of the antibody and the second doses of the antibody) is administered to the subject for a duration of up to about 124 weeks, optionally, once every 4 weeks or once every month. In some embodiments, the anti-N3pGlu Aβ antibody (including the first doses of the antibody and the second doses of the antibody) is administered to the human subject until normal level of amyloid is achieved in the subject. In some embodiments, the antibody is administered to the subject until brain amyloid plaque reaches a normal range or is cleared. In some embodiments, the second doses of the antibody of the present disclosure are administered to the subject until brain amyloid plaque reaches a normal range or is cleared. In some embodiments, the antibody is administered to the subject until the level of brain amyloid plaque stops reducing. In some embodiments, the second doses of the antibody of the present disclosure are administered to the subject until the level of brain amyloid plaque stops reducing. In some embodiments, the antibody is administered to the subject until the subject is amyloid negative. In some embodiments, the second doses of the antibody of the present disclosure are administered to the subject until the subject is amyloid negative. In some embodiments, a subject is considered amyloid negative when the amyloid plaque level in the brain of the subject is less than 24.1 CL. In some embodiments, the level of brain amyloid plaques in a subject can be measured by amyloid PET imaging scan.

In some embodiments, the dose of the anti-N3pGlu Aβ antibody is 700 mg every 4 weeks for the first 3 doses, followed by 1400 mg every 4 weeks, for up to 72 weeks or until brain amyloid plaque reaches a normal range or is cleared. In some embodiments, the dose of the anti-N3pGlu Aβ antibody is 700 mg every 4 weeks for the first 3 doses, followed by 1400 mg every 4 weeks, until brain amyloid plaque stops reducing.

In some embodiments, the anti-N3pGlu Aβ antibody (including the first doses of the antibody and the second doses of the antibody) is administered to the subject for a duration of up to about 18 months, optionally, once every 4 weeks or once every month. In some embodiments, the anti-N3pGlu Aβ antibody (including the first doses of the antibody and the second doses of the antibody) is administered to the subject for a duration of up to about 24 months, optionally, once every 4 weeks or once every month. In some embodiments, the anti-N3pGlu Aβ antibody (including the first doses of the antibody and the second doses of the antibody) is administered to the subject for a duration of up to about 30 months, optionally, once every 4 weeks or once every month.

In one embodiment, the subject is administered three first doses of 700 mg once every four weeks and then second doses of 1400 mg once every four weeks for a duration of up to 72 weeks. In some embodiments, the anti-N3pGlu Aβ antibody (including, e.g., the first doses of the antibody and the second doses of the antibody) is administered to the subject for a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks or about 76 weeks. In some embodiments, the anti-N3pGlu Aβ antibody (including, e.g., the first doses of the antibody and the second doses of the antibody) is administered to the subject for a duration of about 76 weeks, about 80 weeks, about 84 weeks, about 88 weeks, about 92 weeks, about 96 weeks, about 100 weeks, about 104 weeks, about 108 weeks, about 112 weeks, about 116 weeks, or about 120 weeks.

In a particular embodiment, the anti-N3pGlu Aβ antibody is administered to the subject for a duration of about 24 weeks. In a particular embodiment, the antibody is administered to the subject for a duration of about 28 weeks. In a particular embodiment, the antibody is administered to the subject for a duration of about 52 weeks. In a particular embodiment, the antibody is administered to the subject for a duration of about 72 weeks. In some embodiments, the antibody of the present disclosure is administered to the subject for a duration of no more than 72 weeks.

In some embodiments, the anti-N3pGlu Aβ antibody (including, e.g., the first doses of the antibody and the second doses of the antibody) is administered to the subject for a duration of from about 1 month to about 18 months. In some embodiments, the anti-N3pGlu Aβ antibody is administered to the subject for a duration of about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, or about 18 months. In some embodiments, the anti-N3pGlu Aβ antibody is administered to the subject for a duration of about 19 months, about 20 months, about 21 months, about 22 months, about 23 months, about 24 months, about 25 months, about 26 months, about 27 months, about 28 months, about 29 months, or about 30 months.

In some embodiments, the antibody is administered to the subject until brain amyloid plaque reaches a normal range. In some embodiments, the antibody is administered to the subject until brain amyloid plaque is cleared.

In a particular embodiment, the antibody is administered to the subject for a duration of about 3 months. In a particular embodiment, the antibody is administered to the subject for a duration of about 6 months. In a particular embodiment, the antibody is administered to the subject for a duration of about 12 months. In a particular embodiment, the antibody is administered to the subject for a duration of about 18 months.

In some embodiments, the human subject is administered the anti-N3pGlu Aβ antibody for a duration sufficient to treat or prevent the disease characterized by amyloid beta plaques in the brain of the human subject. In some embodiments, the human subject is administered the anti-N3pGlu Aβ antibody (including, e.g., the first dose and/or the second dose) for a duration sufficient to bring the amyloid plaque in the subject's brain to a normal range (or until brain amyloid plaque is cleared). The normal range of amyloid plaque is defined as demonstrating an amyloid plaque level of 25 centiloids or lower for two consecutive PET scans at least 6 months apart or a single PET scan demonstrating a plaque level of less than 11 centiloids. In the present disclosure, the term “normal range” of amyloid plaque in brain is used interchangeably with brain amyloid plaque is “cleared.”

In some embodiments, the antibody of the present disclosure is administered to the subject until amyloid plaque level in the subject is about 25 centiloids or lower. In some embodiments, the amyloid plaque is measured by PET imaging. In other embodiments, the antibody of the present disclosure is administered to the subject until the amyloid plaque level in the subject is about 25 centiloids or lower for two consecutive PET imaging scans. In some embodiments, the two consecutive PET imaging scans are at least 6 months apart. In some embodiments, the antibody of the present disclosure is administered to the subject until the amyloid plaque level in the subject is about 11 centiloids or lower as measured by one PET imaging.

In a particular embodiment, the subject is administered three first doses of 700 mg of the antibody of the present disclosure wherein each first dose is administered once every four weeks and then one or more second doses of 1400 mg of the antibody is administered wherein each second dose is administered once every four weeks until the amyloid plaque level in the patient is about 25 centiloids or lower.

In other embodiments, the subject is administered three first doses of 700 mg of the antibody of the present disclosure wherein each first dose is administered once every four weeks and then second doses of 1400 mg of the antibody is administered wherein each second dose is administered once every four weeks until amyloid plaque level in the patient is about 25 centiloids or lower for two consecutive PET imaging scans or about 11 centiloids or lower for one PET imaging scan. In some embodiments, the two consecutive PET imaging scans are at least 6 months apart.

In some embodiments, the subject is given no anti-N3pGlu Aβ antibody doses after amyloid plaque level in the patient is about 25 centiloids or lower for two consecutive PET imaging scans or about 11 centiloids or lower for one PET imaging scan. In some embodiments, the two consecutive PET imaging scans are at least 6 months apart.

In some embodiments, the subject may be given one or more 700 mg doses of anti-N3pGlu Aβ antibody after amyloid plaque level in the patient is about 25 centiloids or lower for two consecutive PET imaging scans or about 11 centiloids or lower for one PET imaging scan.

In some embodiments, the antibody of the present disclosure is administered to the subject until there is about 25 to about 150 centiloids reduction in amyloid plaque in the brain of the subject. See, e.g., Klunk et al., “The Centiloid Project: Standardizing Quantitative Amyloid Plaque Estimation by PET,” Alzheimer's & Dementia 11.1: 1-15 (2015) and Navitsky et al., “Standardization of Amyloid Quantitation with Florbetapir Standardized Uptake Value Ratios to the Centiloid Scale,” Alzheimer's & Dementia 14.12: 1565-1571 (2018), which are hereby incorporated by reference in their entireties.

In some embodiments, the antibody of the present disclosure is administered to the subject until there is about 50 to about 150 centiloids reduction in Aβ deposit in the brain of the subject. In some embodiments, the antibody of the present disclosure is administered to the subject until there is about 25, about 30, about 40, about 50, about 60, about 70, about 80, about 90, about 100, about 110, about 120, about 130, about 140 or about 150 centiloids reduction in Aβ deposit in the brain of the subject. In some embodiments, the antibody of the present disclosure is administered to the subject until there is about 50 centiloid reduction in Aβ plaques in the brain of the subject. In some embodiments, the antibody of the present disclosure is administered to the subject until there is about 60 centiloid reduction in Aβ plaques in the brain of the subject. In some embodiments, the antibody of the present disclosure is administered to the subject until there is about 70 centiloid reduction in Aβ plaques in the brain of the subject. In some embodiments, the antibody of the present disclosure is administered to the subject until there is about 80 centiloid reduction in Aβ plaques in the brain of the subject. In some embodiments, the antibody of the present disclosure is administered to the subject until there is about 84 centiloid reduction in Aβ plaques in the brain of the subject. In some embodiments, the antibody of the present disclosure is administered to the subject until there is about 90 centiloid reduction in Aβ plaques in the brain of the subject. In some embodiments, the antibody of the present disclosure is administered to the subject until there is about 100 centiloid reduction in Aβ plaques in the brain of the subject. In some embodiments, the antibody of the present disclosure is administered to the subject until there is about 110 centiloid reduction in Aβ plaques in the brain of the subject. In some embodiments, the antibody of the present disclosure is administered to the subject until there is about 120 centiloid reduction in Aβ plaques in the brain of the subject. In some embodiments, the antibody of the present disclosure is administered to the subject until there is about 130 centiloid reduction in Aβ plaques in the brain of the subject. In some embodiments, the antibody of the present disclosure is administered to the subject until there is about 140 centiloid reduction in Aβ plaques in the brain of the subject. In some embodiments, the antibody of the present disclosure is administered to the subject until there is about 150 centiloid reduction in Aβ plaques in the brain of the subject.

In some embodiments, the antibody of the present disclosure is administered to the subject until there is an average of about 25 to about 100 centiloids reduction in Aβ deposit in the brain of the subject. In some embodiments, the antibody of the present disclosure is administered to the subject until there is an average of about 50 to about 100 centiloids reduction in Aβ deposit in the brain of the subject. In some embodiments, the antibody of the present disclosure is administered to the subject until there is an average of about 10, about 20, about 30, about 40, about 50, about 60, about 70, about 80, about 84, about 90, about 100 centiloids reduction in Aβ deposit in the brain of the subject. In some embodiments, the antibody of the present disclosure is administered to the subject until there is an average of about 50 centiloid reduction in Aβ plaques in the brain of the subject. In some embodiments, the antibody of the present disclosure is administered to the subject until there is an average of about 60 centiloid reduction in Aβ plaques in the brain of the subject. In some embodiments, the antibody of the present disclosure is administered to the subject until there is an average of about 70 centiloid reduction in Aβ plaques in the brain of the subject. In some embodiments, the antibody of the present disclosure is administered to the subject until there is an average of about 80 centiloid reduction in Aβ plaques in the brain of the subject. In some embodiments, the antibody of the present disclosure is administered to the subject until there is an average of about 84 centiloid reduction in Aβ plaques in the brain of the subject. In some embodiments, the antibody of the present disclosure is administered to the subject until there is an average of about 90 centiloid reduction in Aβ plaques in the brain of the subject. In some embodiments, the antibody of the present disclosure is administered to the subject until there is an average of about 100 centiloid reduction in Aβ plaques in the brain of the subject.

In some embodiments, the second dose of the antibody of the present disclosure is administered to the subject until there is about 25 to about 150 centiloids reduction in Aβ deposit in the brain of the subject. In some embodiments, the second dose of the antibody of the present disclosure is administered to the subject until there is about 50 to about 150 centiloids reduction in Aβ deposit in the brain of the subject. In some embodiments, the second dose of the antibody of the present disclosure is administered to the subject until there is about 25, about 30, about 40, about 50, about 60, about 70, about 80, about 84, about 90, about 100, about 110, about 120, about 130, about 140 or about 150 centiloids reduction in Aβ deposit in the brain of the subject. In some embodiments, the second dose of the antibody of the present disclosure is administered to the subject until there is about 50 centiloid reduction in Aβ plaques in the brain of the subject. In some embodiments, the second dose of the antibody of the present disclosure is administered to the subject until there is about 60 centiloid reduction in Aβ plaques in the brain of the subject. In some embodiments, the second dose of the antibody of the present disclosure is administered to the subject until there is about 70 centiloid reduction in Aβ plaques in the brain of the subject. In some embodiments, the second dose of the antibody of the present disclosure is administered to the subject until there is about 80 centiloid reduction in Aβ plaques in the brain of the subject. In some embodiments, the second dose of the antibody of the present disclosure is administered to the subject until there is about 84 centiloid reduction in Aβ plaques in the brain of the subject. In some embodiments, the second dose of the antibody of the present disclosure is administered to the subject until there is about 90 centiloid reduction in Aβ plaques in the brain of the subject. In some embodiments, the second dose of the antibody of the present disclosure is administered to the subject until there is about 100 centiloid reduction in Aβ plaques in the brain of the subject. In some embodiments, the second dose of the antibody of the present disclosure is administered to the subject until there is about 110 centiloid reduction in Aβ plaques in the brain of the subject. In some embodiments, the second dose of the antibody of the present disclosure is administered to the subject until there is about 120 centiloid reduction in Aβ plaques in the brain of the subject. In some embodiments, the second dose of the antibody of the present disclosure is administered to the subject until there is about 130 centiloid reduction in Aβ plaques in the brain of the subject. In some embodiments, the second dose of the antibody of the present disclosure is administered to the subject until there is about 140 centiloid reduction in Aβ plaques in the brain of the subject. In some embodiments, the second dose of the antibody of the present disclosure is administered to the subject until there is about 150 centiloid reduction in Aβ plaques in the brain of the subject.

In some embodiments, the second dose of the antibody of the present disclosure is administered to the subject until there is an average of about 25 to about 100 centiloids reduction in Aβ deposit in the brain of the subject. In some embodiments, the second dose of the antibody of the present disclosure is administered to the subject until there is an average of about 50 to about 100 centiloids reduction in Aβ deposit in the brain of the subject. In some embodiments, the second dose of the antibody of the present disclosure is administered to the subject until there is an average of about 25, about 30, about 40, about 50, about 60, about 70, about 80, about 84, about 90, about 100 centiloids reduction in Aβ deposit in the brain of the subject. In some embodiments, the second dose of the antibody of the present disclosure is administered to the subject until there is an average of about 50 centiloid reduction in Aβ plaques in the brain of the subject. In some embodiments, the second dose of the antibody of the present disclosure is administered to the subject until there is an average of about 60 centiloid reduction in Aβ plaques in the brain of the subject. In some embodiments, the second dose of the antibody of the present disclosure is administered to the subject until there is an average of about 70 centiloid reduction in Aβ plaques in the brain of the subject. In some embodiments, the second dose of the antibody of the present disclosure is administered to the subject until there is an average of about 80 centiloid reduction in Aβ plaques in the brain of the subject. In some embodiments, the second dose of the antibody of the present disclosure is administered to the subject until there is an average of about 84 centiloid reduction in Aβ plaques in the brain of the subject. In some embodiments, the second dose of the antibody of the present disclosure is administered to the subject until there is an average of about 90 centiloid reduction in Aβ plaques in the brain of the subject. In some embodiments, the second dose of the antibody of the present disclosure is administered to the subject until there is an average of about 100 centiloid reduction in Aβ plaques in the brain of the subject.

In some embodiments, the antibodies, methods, dosing regimens, and/or uses of the present disclosure result in reduction of Aβ plaques in the brain of a human subject. In particular embodiments, the Aβ plaques are reduced by about 20-100% post treatment. In some embodiments, the antibody of the present disclosure is administered to the subject until there is about 20-100% reduction in Aβ plaques in the brain of the subject. In some embodiments, the antibody of the present disclosure is administered to the subject until the Aβ plaques in the brain of the subject are reduced by about 20%, about 25%, about 30%, about 35%, about 40%, about 45%, about 50%, about 75% or about 100%. In some embodiments, the antibody of the present disclosure is administered to the subject until there is about 20% reduction in Aβ plaques in the brain of the subject. In some embodiments, the antibody of the present disclosure is administered to the subject until there is about 25% reduction in Aβ plaques in the brain of the subject. In some embodiments, the antibody of the present disclosure is administered to the subject until there is about 30% reduction in Aβ plaques in the brain of the subject. In some embodiments, the antibody of the present disclosure is administered to the subject until there is about 35% reduction in Aβ plaques in the brain of the subject. In some embodiments, the antibody of the present disclosure is administered to the subject until there is about 40% reduction in Aβ plaques in the brain of the subject. In some embodiments, the antibody of the present disclosure is administered to the subject until there is about 50% reduction in Aβ plaques in the brain of the subject. In some embodiments, the antibody of the present disclosure is administered to the subject until there is about 75% reduction in Aβ plaques in the brain of the subject. In some embodiments, the antibody of the present disclosure is administered to the subject until there is about 100% reduction in Aβ plaques in the brain of the subject.

In some embodiments, the first dose and/or the second dose of antibody of the present disclosure are administered to the subject until the Aβ plaques in the brain of the subject are reduced by about 20-100%. In particular embodiments, the second doses of antibody of the present disclosure are administered to the subject until the Aβ plaques in the brain of the subject are reduced by about 20-100%. In some embodiments, the second doses of the antibody of the present disclosure are administered to the subject until the Aβ plaques in the brain of the subject are reduced by about 20%, about 25%, about 30%, about 35%, about 40%, about 45%, about 50%, about 75% or about 100%. In some embodiments, the second doses are administered to the subject until there is about 20% reduction in Aβ plaques in the brain of the subject. In some embodiments, the second doses are administered to the subject until there is about 25% reduction in Aβ plaques in the brain of the subject. In some embodiments, the second doses are administered to the subject until there is about 30% reduction in Aβ plaques in the brain of the subject. In some embodiments, the second doses are administered to the subject until there is about 35% reduction in Aβ plaques in the brain of the subject. In some embodiments, the second doses are administered to the subject until there is about 40% reduction in Aβ plaques in the brain of the subject. In some embodiments, the second doses are administered to the subject until there is about 50% reduction in Aβ plaques in the brain of the subject. In some embodiments, the second doses are administered to the subject until there is about 75% reduction in Aβ plaques in the brain of the subject. In some embodiments, the second doses are administered to the subject until there is about 100% reduction in Aβ plaques in the brain of the subject.

In some embodiments, the percentage reduction in Aβ plaques in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks. In some embodiments, Aβ plaque level in a subject is reduced by at least 60% within 24 weeks of administration of the anti-N3pGlu Aβ antibody (including both the first doses and the second doses) of the present invention.

In some embodiments, the centiloids reduction in Aβ plaques in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the average centiloids reduction in Aβ plaques in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the present disclosure results in about 15 to about 45 percent slowing of decline in the cognitive-functional composite endpoints from baseline. In some embodiments, the present disclosure results in about 15 to about 45 percent slowing of decline in the cognitive-functional composite endpoints from baseline over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks.

In some embodiments, the present disclosure results in about 15 to about 45 percent slowing of decline in the cognitive-functional composite endpoints from baseline over a duration of 76 weeks. In some embodiments, the slowing of decline in the cognitive-functional composite endpoints from baseline is provided from the mixed-model repeated-measures (MMRM) model or the Bayesian Disease Progression Model (DPM). In some embodiments, the antibody of the present disclosure is administered to the subject till it reaches about 15 to about 45 percent slowing of decline in the cognitive-functional composite endpoints from baseline. In some embodiments, the first or the second dose of the present disclosure is administered to the subject till it reaches about 15 to about 45 percent slowing of decline in the cognitive-functional composite endpoints from baseline.

In some embodiments, the administration of the anti-N3pGlu Aβ antibody of the present disclosure to the subject slows disease progression by about 15% to about 45% as compared to untreated subject, wherein the disease progression is measured by DPM. In some embodiments, the administration of the anti-N3pGlu Aβ antibody of the present disclosure to the subject slows disease progression by at least 15% as compared to untreated subject, wherein the disease progression is measured by DPM. In some embodiments, the administration of the anti-N3pGlu Aβ antibody of the present disclosure to the subject slows disease progression by at least 20% as compared to untreated subject, wherein the disease progression is measured by DPM. In some embodiments, the administration of the anti-N3pGlu Aβ antibody of the present disclosure to the subject slows disease progression by at least 25% as compared to untreated subject, wherein the disease progression is measured by DPM. In some embodiments, the administration of the anti-N3pGlu Aβ antibody of the present disclosure to the subject slows disease progression by at least 15%, at least 20%, at least 25%, at least 30%, at least 35%, at least 40% or at least 45% as compared to untreated subject, wherein the disease progression is measured by DPM.

In some embodiments, the administration of the anti-N3pGlu Aβ antibody of the present disclosure to the subject slows disease progression by about 15% to about 45% as compared to untreated subject, wherein the disease progression is measured by MMRM. In some embodiments, the administration of the anti-N3pGlu Aβ antibody of the present disclosure to the subject slows disease progression by at least 15% as compared to untreated subject, wherein the disease progression is measured by MMRM. In some embodiments, the administration of the anti-N3pGlu Aβ antibody of the present disclosure to the subject slows disease progression by at least 20% as compared to untreated subject, wherein the disease progression is measured by MMRM. In some embodiments, the administration of the anti-N3pGlu Aβ antibody of the present disclosure to the subject slows disease progression by at least 25% as compared to untreated subject, wherein the disease progression is measured by MMRM. In some embodiments, the administration of the anti-N3pGlu Aβ antibody of the present disclosure to the subject slows disease progression by at least 15%, at least 20%, at least 25%, at least 30%, at least 35%, at least 40% or at least 45% as compared to untreated subject, wherein the disease progression is measured by MMRM.

In some embodiments, the present disclosure results in about 15 to about 60 percent slowing of decline or disease progression on the Integrated Alzheimer's Disease Rating Scale (iADRS) from baseline or as compared to untreated subject. In some embodiments, the present disclosure results in about 15 to about 60 percent slowing of decline or disease progression on the Integrated Alzheimer's Disease Rating Scale from baseline or as compared to untreated subject over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks. In some embodiments, the slowing of decline as measured by iADRS is provided from the mixed-model repeated-measures (MMRM) model or the Bayesian Disease Progression Model (DPM).

In some embodiments, the present disclosure results in about 20 percent, about 25 percent, about 30 percent, about 32 percent, about 35 percent, about 40 percent, about 45 percent, about 50%, about 55%, or about 60% slowing of decline or disease progression in the Integrated Alzheimer's Disease Rating Scale from baseline or as compared to untreated subject.

In some embodiments, the present disclosure results in about 15 to about 60 percent slowing of decline on the Integrated Alzheimer's Disease Rating Scale from baseline or as compared to untreated subject over a duration of 76 weeks. In a particular embodiment, the present disclosure results in about 32 percent slowing of decline on the Integrated Alzheimer's Disease Rating Scale from baseline or as compared to untreated subject over a duration of 76 weeks. In some embodiments, the antibody of the present disclosure is administered to the subject till it reaches about 15 to about 60 percent slowing of decline on the Integrated Alzheimer's Disease Rating Scale from baseline or as compared to untreated subject. In some embodiments, the first or the second dose of the present disclosure is administered to the subject till it reaches about 15 to about 60 percent slowing of decline on the Integrated Alzheimer's Disease Rating Scale from baseline or as compared to untreated subject.

In some embodiments, the present disclosure results in about 3 to about 6 slowing of decline or disease progression on the Integrated Alzheimer's Disease Rating Scale (iADRS) from baseline or as compared to untreated subject. In some embodiments, the present disclosure results in about 3 to about 6 slowing of decline or disease progression on the Integrated Alzheimer's Disease Rating Scale from baseline or as compared to untreated subject over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks.

In some embodiments, the present disclosure results in about 3, about 4, about 5, or about 6 slowing of decline or disease progression in the Integrated Alzheimer's Disease Rating Scale from baseline or as compared to untreated subject. In some embodiments, the present disclosure results in 3 to about 6 points slowing of decline or disease progression on the Integrated Alzheimer's Disease Rating Scale from baseline or as compared to untreated over a duration of 76 weeks.

In some embodiments, the slowing of disease progression as measured by iADRS is provided from the mixed-model repeated-measures (MMRM) model or the Bayesian Disease Progression Model (DPM).

In some embodiments, the cognitive functional composite endpoint, including iADRS, of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the present disclosure results in about 20 to about 40 percent slowing of decline or disease progression on Clinical Dementia Rating Scale—Sum of Boxes (CDR-SB) from baseline or as compared to untreated subject. In some embodiments, the present disclosure results in about 20 to about 40 percent slowing of decline or disease progression on CDR-SB from baseline or as compared to untreated over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks.

In some embodiments, the present disclosure results in about 20 percent, about 25 percent, about 30 percent, about 35 percent, or about 40 percent slowing of decline or disease progression on CDR-SB from baseline or as compared to untreated subject.

In some embodiments, the present disclosure results in about 20 to about 40 percent slowing of decline on CDR-SB from baseline or as compared to untreated subject over a duration of 76 weeks. In some embodiments, the antibody of the present disclosure is administered to the subject till it reaches about 20 to about 40 percent slowing of decline or disease progression on CDR-SB from baseline or as compared to untreated subject. In some embodiments, the first or the second dose of the present disclosure is administered to the subject till it reaches about 20 to about 40 percent slowing of decline or disease progression on CDR-SB from baseline or as compared to untreated subject. In some embodiments, the slowing of disease progression as measured by CDR-SB is provided from the mixed-model repeated-measures (MMRM) model or the Bayesian Disease Progression Model (DPM).

In some embodiments, the antibodies, methods, dosing regimens, and/or uses of the present disclosure do not result in reduction of the subject's hippocampal volume. In some embodiments, administration of the antibody does not result in reduction of the subject's hippocampal volume.

In some embodiments, the antibodies, methods, dosing regimens, and/or uses of the present disclosure result in decrease or reduction in the tau levels in brain of the human subject. In some embodiments, the antibodies, methods, dosing regimens, and/or uses of the present disclosure result in decrease or reduction in plasma tau levels in the patient having a disease characterized by Aβ plaques. In some embodiments, the antibodies, methods, dosing regimens, and/or uses of the present disclosure result in decrease or reduction in P-tau 217 levels in a patient having a disease characterized by Aβ plaques. In some embodiments, the antibodies, methods, dosing regimens, and/or uses of the present disclosure result in rapid and sustained reduction in P-tau 217 levels in a patient having a disease characterized by Aβ plaques. In some embodiments, the P-tau 217 levels are reduced from about 5% to about 40% from baseline. In some embodiments, the P-tau 217 levels are reduced from about 10% to about 30% from baseline. In some embodiments, the P-tau 217 levels are reduced from about 20% to about 30% from baseline. In some embodiments, the P-tau 217 levels are reduced from about 25% to about 30% from baseline. In some embodiments, the P-tau 217 levels are reduced by 5%, 10%, 15%, 20%, 24%, 25%, 29%, 30%, 35%, or 40% from baseline. In some embodiments, the P-tau 217 levels are reduced from about 5% to about 40% from baseline post-treatment with the anti-N3pG antibody. In some embodiments, the P-tau 217 levels are reduced from about 10% to about 30% from baseline post-treatment with the anti-N3pG antibody. In some embodiments, the P-tau 217 levels are reduced from about 20% to about 30% from baseline post-treatment with the anti-N3pG antibody. In some embodiments, the P-tau 217 levels are reduced from about 25% to about 30% from baseline post-treatment with the anti-N3pG antibody. In some embodiments, the P-tau 217 levels are reduced by 5%, 10%, 15%, 20%, 24%, 25%, 29%, 30%, 35%, or 40% from baseline post-treatment with the anti-N3pG antibody. In some embodiments, the P-tau 217 levels are reduced from about 5% to about 40% from baseline at one or more time points during or after treatment with the anti-N3pG antibody of the present disclosure.

In some embodiments, the antibodies, methods, dosing regimens, and/or uses of the present disclosure result in decrease or reduction in Neurofilament light chain (NfL) levels in brain of the patient having a disease characterized by Aβ plaques. In some embodiments, the NfL levels are reduced from about 1% to about 20% as compared to placebo. In some embodiments, the NfL levels are reduced from about 5% to about 15% as compared to placebo. In some embodiments, the NfL levels are reduced from about 10% to about 15% as compared to placebo. In some embodiments, the NfL levels are reduced from by 2%, 3%, 4%, 5%, 10%, 15%, or 20% as compared to placebo. In some embodiments, the NfL levels are reduced from about 1% to about 20% as compared to placebo post-treatment with the anti-N3pG antibody. In some embodiments, the NfL levels are reduced from about 5% to about 15% as compared to placebo post-treatment with the anti-N3pG antibody. In some embodiments, the NfL levels are reduced from about 10% to about 15% as compared to placebo post-treatment with the anti-N3pG antibody. In some embodiments, the NfL levels are reduced from by 2%, 3%, 4%, 5%, 10%, 15%, or 20% as compared to placebo post-treatment with the anti-N3pG antibody. In some embodiments, the NfL levels are reduced from by 2%, 3%, 4%, 5%, 10%, 15%, or 20% as compared to placebo post-treatment with the anti-N3pG antibody. In some embodiments, the NfL levels are reduced from about 1% to about 20% as compared to placebo at one or more time points during or after treatment with the anti-N3pG antibody of the present disclosure.

In some embodiments, the antibodies, methods, dosing regimens, and/or uses of the present disclosure result in increase in Aβ_{42/40} ratio in plasma or cerebrospinal fluid (CSF) of the patient having a disease characterized by Aβ plaques. In some embodiments, the Aβ_{42/40} ratio in plasma is increased from about 1% to about 10% as compared to baseline. In some embodiments, the Aβ_{42/40} ratio in plasma is increased from about 1% to about 5% as compared to baseline. In some embodiments, the Aβ_{42/40} ratio in plasma is increased by 1%, 2%, 3%, 4%, 5%, 6%, 7%, 8%, 9%, or 10%, as compared to baseline. In some embodiments, the Aβ_{42/40} ratio in plasma is increased from about 1% to about 10% as compared to baseline post-treatment with the anti-N3pG antibody. In some embodiments, the Aβ_{42/40} ratio in plasma is increased from about 1% to about 5% as compared to baseline post-treatment with the anti-N3pG antibody. In some embodiments, the Aβ_{42/40} ratio in plasma is increased by 1%, 2%, 3%, 4%, 5%, 6%, 7%, 8%, 9%, or 10%, as compared to baseline post-treatment with the anti-N3pG antibody. In some embodiments, the Aβ_{42/40} ratio in plasma is increased from about 1% to about 10% as compared to baseline at one or more time points during or after treatment with the anti-N3pG antibody of the present disclosure.

In some embodiments, the antibodies, methods, dosing regimens, and/or uses of the present disclosure result in decrease or reduction in glial fibrillary acidic protein (GFAP) in blood of the patient having a disease characterized by Aβ plaques. In some embodiments, the GFAP levels are reduced from about 5% to about 40% from baseline. In some embodiments, the GFAP levels are reduced from about 10% to about 30% from baseline. In some embodiments, the GFAP levels are reduced from about 10% to about 20% from baseline. In some embodiments, the GFAP levels are reduced from about 10% to about 15% from baseline. In some embodiments, the GFAP levels are reduced by 5%, 10%, 12%, 15%, 20%, 24%, 25%, 29%, 30%, 35%, or 40% from baseline. In some embodiments, the GFAP levels are reduced from about 5% to about 40% from baseline post-treatment with the anti-N3pG antibody. In some embodiments, the GFAP levels are reduced from about 10% to about 30% from baseline post-treatment with the anti-N3pG antibody. In some embodiments, the GFAP levels are reduced from about 10% to about 20% from baseline post-treatment with the anti-N3pG antibody. In some embodiments, the GFAP levels are reduced from about 10% to about 15% from baseline post-treatment with the anti-N3pG antibody. In some embodiments, the GFAP levels are reduced by 5%, 10%, 12%, 15%, 20%, 24%, 25%, 29%, 30%, 35%, or 40% from baseline post-treatment with the anti-N3pG antibody. In some embodiments, the GFAP levels are reduced from about 5% to about 40% from baseline at one or more time points during or after treatment with the anti-N3pG antibody of the present disclosure. In some embodiments, the GFAP levels are reduced from about 5% to about 30% from baseline at one or more time points during or after treatment with the anti-N3pG antibody of the present disclosure. In some embodiments, the GFAP levels are reduced from about 5% to about 20% from baseline at one or more time points during or after treatment with the anti-N3pG antibody of the present disclosure. In some embodiments, the GFAP levels are reduced from about 5% to about 15% from baseline at one or more time points during or after treatment with the anti-N3pG antibody of the present disclosure. In some embodiments, the GFAP levels are reduced by 5%, 10%, 14%, 15%, 20%, 25%, 30%, 35%, or 40% from baseline at one or more time points during or after treatment with the anti-N3pG antibody of the present disclosure.

In some embodiments, the antibody of the present disclosure can be administered in simultaneous, separate, or sequential combination with an effective amount of a symptomatic agent to treat Alzheimer's disease. Symptomatic agents can be selected from cholinesterase inhibitors (ChEIs) and/or a partial N-methyl-D-aspartate (NMDA) antagonists. In a preferred embodiment the agent is a ChEI. In another preferred embodiment the agent is a NMDA antagonist or a combination agent comprising a ChEI and NMDA antagonist.

In some embodiments, the dosing regimen or the methods described herein include a step of administering solanezumab or an antibody comprising portions of solanezumab to the human patient. In some embodiments, the anti-N3pGlu Aβ antibody is administered in simultaneous, separate, or sequential combination with an effective amount of antibody having a light chain of SEQ ID NO: 15. In some embodiments, the anti-N3pGlu Aβ antibody is administered in simultaneous, separate, or sequential combination with an effective amount of antibody having a heavy chain of SEQ ID NO: 16. In some embodiments, the anti-N3pGlu Aβ antibody is administered in simultaneous, separate, or sequential combination with an effective amount of antibody having two heavy chains of SEQ ID NO: 16 and two light chains of SEQ ID NO: 15. In some embodiments, the anti-N3pGlu Aβ antibody of the present disclosure can be administered in simultaneous, separate, or sequential combination with an effective amount of solanezumab.

Additional information about solanezumab, including its CDR sequences, LCVR, HCVR sequences, and methods of making and using it can be found in the following patent documents, which are hereby incorporated by reference in their entirety:
U.S. Pat. No. 7,195,761
US Patent Application Publication No. 20060039906
U.S. Pat. No. 7,892,545
U.S. Pat. No. 8,591,894
U.S. Pat. No. 7,771,722
US Patent Application Publication No. 20070190046.

Information about using solanezumab in combination with other antibodies is found in US Patent Application Publication No. 201903824, which is hereby incorporated by reference in its entirety.

In some embodiments, solanezumab or an antibody comprising portions of solanezumab is administered to the human subject to maintain amyloid beta levels within a normal range. In embodiments, solanezumab or an antibody comprising portions of solanezumab is administered to the human subject to prevent an increase in amyloid plaque levels. In embodiments, solanezumab or an antibody comprising portions of solanezumab is administered to the human subject to reduce the rate of increase of amyloid plaque levels.

In some embodiments, the human subject may be administered dose(s) or dosing regimens for an anti-N3pGlu Aβ antibody, as described herein, in combination with dose(s) or dosing regimen for solanezumab or an antibody comprising portions of solanezumab. In some embodiments, the dose of solanezumab is 400 mg every 4 weeks, 800 mg every 4 weeks, 1200 mg every 4 weeks or 1600 mg every 4 weeks. In some embodiments, the dosing regimen of solanezumab comprises an initial dose of 400 mg, and either maintaining the patient at 400 mg or titrating up to 800 mg every 4 weeks or titrating up 1200 mg every 4 weeks or titrating up 1600 mg over time. Other embodiments may involve giving an initial dose of 1600 mg and then maintaining at that dose or titrating down to 400 mg, 800 mg, or 1200 mg. Those skilled in the art will appreciate how to titrate up or down with the dose, or to maintain the patient on a particular dose (and the timing associated with making a dosing change).

In some embodiments, the administration of solanezumab causes a reduction in the soluble Aβ that is available in the brain. This reduction may be measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks or about 80 weeks.

In some embodiments, the administration of solanezumab results in a 5% lowering of the soluble Aβ concentration. In other embodiments, administration of solanezumab results in a 10% lowering of the soluble Aβ concentration. In other embodiments, administration of solanezumab results in a 15% lowering of the soluble Aβ concentration. In other embodiments, administration of solanezumab results in a 20% lowering of the soluble Aβ concentration. In other embodiments, administration of solanezumab results in a 25% lowering of the soluble Aβ concentration. In other embodiments, administration of solanezumab results in a 30% lowering of the soluble Aβ concentration. In other embodiments, administration of solanezumab results in a 35% lowering of the soluble Aβ concentration. In other embodiments, administration of solanezumab results in a 40% lowering of the soluble Aβ concentration. In other embodiments, administration of solanezumab results in a 45% lowering of the soluble Aβ concentration. In other embodiments, administration of solanezumab results in a 50% lowering of the soluble Aβ concentration. In other embodiments, administration of solanezumab results in a greater than 50% lowering of the soluble Aβ concentration. Those skilled in the art will appreciate how to measure the concentration of the soluble Aβ concentration. See Siemers et al., “Safety and Changes in Plasma and Cerebrospinal Fluid Amyloid β After a Single Administration of an Amyloid β Monoclonal Antibody in Subjects with Alzheimer Disease.” Clinical Neuropharmacology 33.2 (2010): 67-73 and Farlow et al., “Safety and Biomarker Effects of Solanezumab in Patients with Alzheimer's Disease,” Alzheimer's & Dementia 8.4 (2012): 261-271, each of which is hereby incorporated by reference in its entirety.

Those skilled in the art will appreciate that changing the dose of solanezumab or another antibody may be based upon various factors, including PET scans, clinical observations, performance by the patient on various “tests”, etc.

In some embodiments, the disease characterized by Aβ deposit in the brain of the subject is selected from preclinical Alzheimer's disease, clinical AD, prodromal AD, mild AD, moderate AD, severe AD, Down's syndrome, clinical cerebral amyloid angiopathy, or pre-clinical cerebral amyloid angiopathy. In some embodiments, the subject is an early symptomatic AD patient. In some embodiments, the subject has prodromal AD and mild dementia due to AD.

The present disclosure includes use of biomarkers of a disease characterized by Aβ plaques in the brain of a human subject, including Alzheimer's disease. Such biomarkers include, e.g., amyloid deposits, amyloid plaque, Aβ in CSF, Aβ in the plasma, brain tau deposition, tau in plasma, or tau in cerebrospinal fluid and their use in screening, diagnosis, treatment, or prevention. Non-limiting potential uses of such biomarkers include: 1) identification of subjects destined to become affected or who are in the “preclinical” stages of a disease; 2) reduction in disease heterogeneity in clinical trials or epidemiologic studies; 3) reflection of the natural history of disease encompassing the phases of induction, latency, and detection; and 4) target subjects for a clinical trial or for treatment/prevention of a disease.

In some embodiments, the biomarkers may be used to assess whether a subject can be treated using the antibodies, the dosing regimen, or the methods described herein. In some embodiments, the biomarkers may be used to assess whether a disease (as described herein) can be prevented in the subject using the antibodies, the dosing regimen, or the methods described herein. In some embodiments, the biomarkers can be used to assess whether a subject is responsive to treatment or prevention of a disease (as described herein) using the antibodies, the dosing regimen, or the methods described herein. In some embodiments, the biomarkers can be used to stratify or classify subjects into groups and to identify which group of subjects is responsive to treatment/prevention of diseases (as described herein) using the antibodies, the dosing regimen, or the methods described herein. In some embodiments, the biomarkers may be used to assess disease state of a subject and/or the duration for administration of the antibodies or doses thereof, as described herein, to the subject.

In some embodiments, the subject has a genetic mutation that causes autosomal-dominant Alzheimer's disease or at a higher risk for developing AD by virtue of carrying one or two APOE4 alleles. In particular embodiments, the subject carries one or two APOE4 alleles, i.e., the patient is heterozygous or homozygous.

In some embodiments, the subject has a baseline MMSE (Mini-Mental State Exam) score of 20 to 28 prior to administering the anti-N3pGlu Aβ antibody.

In some embodiments, the subject has low to moderate tau burden or has been determined to have low to moderate tau burden. The subject has low to moderate tau burden if the tau burden as measured by PET brain imaging (using, e.g., ¹⁸F flortaucipir) is from ≥1.10 standardized uptake value ratio (SUVr) to ≤1.46 SUVr. In some embodiments, the subject has low to moderate tau burden or has been determined to have low to moderate tau burden and carries one or two APOE4 alleles.

In some embodiments, the subject has very low tau burden or has been determined to have very low tau burden. The subject has very low tau burden if the tau burden as measured by PET brain imaging (using, e.g., ¹⁸F flortaucipir) is less than 1.10 SUVr. In some embodiments, the subject has very low tau burden or has been determined to have very low tau burden and carries one or two APOE4 alleles.

In some embodiments, the subject has very low to moderate tau burden or has been determined to have very low tau to moderate tau burden. The subject has very low to moderate tau burden if the tau burden as measured by PET brain imaging (using, e.g., ¹⁸F flortaucipir) is ≤1.46 SUVr. In some embodiments, the subject has very low to moderate tau burden or has been determined to have very low to moderate tau burden and carries one or two APOE4 alleles.

In some embodiments, the subject does not have a high tau burden or has been determined to not have a high tau burden. In some embodiments, the human subject has high tau burden if the tau burden as measured by PET brain imaging (using, e.g., ¹⁸F flortaucipir) is greater than 1.46 SUVr. In some embodiments, a subject with high tau is not administered the antibodies of the present disclosure. In some embodiments, the subject has does not have high tau burden or has been determined to not have a high tau burden and carries one or two APOE4 alleles.

In some embodiments, the anti-N3pGlu Aβ antibody, the dosing regimen, or the method described the present disclosure is efficacious in human subjects having very low to moderate tau. In some embodiments, the anti-N3pGlu Aβ antibody, the dosing regimen, or the method described the present disclosure is efficacious in human subjects having low to moderate tau. In some embodiments, the antibody of the present disclosure is most efficacious in human subjects having a tau level i) less than or equal to about 1.14 SUVr or ii) from about 1.14 SUVr to about 1.27 SUVr. In some embodiments, the anti-N3pGlu Aβ antibody, the dosing regimen, or the method described the present disclosure is efficacious in human subjects irrespective of their tau levels.

In some embodiments, the anti-N3pGlu Aβ antibody, the dosing regimen, or the method described the present disclosure is efficacious in human subjects having very low to moderate tau and carrying one or two APOE4 alleles. In some embodiments, the anti-N3pGlu Aβ antibody, the dosing regimen, or the method described the present disclosure is efficacious in human subjects having low to moderate tau and carrying one or two APOE4 alleles. In some embodiments, the antibody of the present disclosure is most efficacious in human subjects carrying one or two APOE4 alleles and having a tau level i) less than or equal to about 1.14 SUVr or ii) from about 1.14 SUVr to about 1.27 SUVr.

In some embodiments, the methods of the present disclosure are such that the level of Aβ plaques in the brain of the subject is sustained at normal levels for at least 52 weeks after completing administration of the second dose.

The tau level of a human subject can be determined by techniques and methods familiar to the diagnosing physician or a person of ordinary skill in the art. In some embodiments, a human subject, who is suffering from a disease characterized by amyloid beta plaques, is determined to have very low to moderate tau, low to moderate tau, or no high tau using techniques and methods familiar to the diagnosing physician or a person of ordinary skill in the art. In some embodiments, such methods can also be used to prescreen, screen, diagnose, evaluate increase or reduction in brain tau burden, and/or to assess the progress achieved in the treatment or prevention of the diseases described herein. In some embodiments, the methods can also be used to stratify subjects into groups and/or to identify which group of subjects is responsive to treatment/prevention of a disease (as described herein) using the antibodies, the dosing regimen, or the methods described herein. In some embodiments, the methods or techniques used to determine/detect tau level of a human subject can be used for prescreening or screening subjects and determining which subjects are responsive to treatment/prevention of a disease (as described herein) using the antibodies, the dosing regimen, or the methods described herein.

In some embodiments, the tau level of a human subject can be determined using techniques or methods that, e.g., detect or quantitate i) brain tau deposition, ii) tau in plasma, or iii) tau in cerebrospinal fluid. In some embodiments, brain tau burden, tau in plasma, or tau in cerebrospinal fluid can be used to stratify subjects into groups and to identify which group of subjects is responsive to treatment/prevention of diseases (described herein) using the antibodies, the dosing regimen, or the methods described herein.

Tau levels in the brain of human subject can be determined using methods, such as, tau imaging with radiolabeled PET compounds (Leuzy et al., “Diagnostic Performance of RO948 F18 Tau Positron Emission Tomography in the Differentiation of Alzheimer Disease from Other Neurodegenerative Disorders,” JAMA Neurology 77.8:955-965 (2020); Ossenkoppele et al., “Discriminative Accuracy of F18-flortaucipir Positron Emission Tomography for Alzheimer Disease vs Other Neurodegenerative Disorders,” JAMA 320, 1151-1162, doi:10.1001/jama.2018.12917 (2018), which are hereby incorporated by reference in their entireties.

In some embodiments, the biomarker F18-flortaucipir, which is a PET ligand, may be used for the purposes of the present disclosure. PET tau images can be, for example, quantitatively evaluated to estimate an SUVr (standardized uptake value ratio) by published methods (Pontecorvo et al., “A Multicentre Longitudinal Study of Flortaucipir (¹⁸F) in Normal Ageing, Mild Cognitive Impairment and Alzheimer's Disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and/or to visually evaluate patients, e.g., to determine whether the patient has an AD pattern (Fleisher et al., “Positron Emission Tomography Imaging With F18-flortaucipir and Postmortem Assessment of Alzheimer Disease Neuropathologic Changes,” JAMA Neurology 77:829-39 (2020), which is hereby incorporated by reference in its entirety). Lower SUVr values indicate less tau burden while higher SUVr values indicate a higher tau burden. In an embodiment, quantitative assessment by a flortaucipir scan is accomplished through an automated image processing pipeline as described in Southekal et al., “Flortaucipir F18 Quantitation Using Parametric Estimation of Reference Signal Intensity,” J. Nucl. Med. 59:944-951 (2018), which is hereby incorporated by reference in its entirety. In some embodiments, counts within a specific target region of interest in the brain (e.g., multiblock barycentric discriminant analysis or MUBADA, see Devous et al, “Test-Retest Reproducibility for the Tau PET Imaging Agent Flortaucipir F18,” J. Nucl. Med. 59:937-943 (2018), which is hereby incorporated by reference in its entirety) are compared with a reference region wherein the reference region is, e.g., whole cerebellum, (wholeCere), cerebellar GM (cereCrus), atlas-based white matter (atlasWM), subject-specific WM (ssWM, e.g., using parametric estimate of reference signal intensity (PERSI), see Southekal et al., “Flortaucipir F18 Quantitation Using Parametric Estimation of Reference Signal Intensity,” J. Nucl. Med. 59:944-951 (2018), which is hereby incorporated by reference in its entirety). A preferred method of determining tau burden is a quantitative analysis reported as a standardized uptake value ratio (SUVr), which represents counts within a specific target region of interest in the brain (e.g., MUBADA,) when compared with a reference region (e.g., using PERSI).

In some embodiments, phosphorylated tau (P-tau; either phosphorylated at threonine 181 or 217) can be used to measure the tau load/burden for the purposes of the present disclosure (Barthelemy et al., “Cerebrospinal Fluid Phospho-tau T217 Outperforms T181 as a Biomarker for the Differential Diagnosis of Alzheimer's Disease and PET Amyloid-positive Patient Identification,” Alzheimer's Res. Ther. 12, 26, doi:10.1186/s13195-020-00596-4 (2020); Mattsson et al., “Aβ Deposition is Associated with Increases in Soluble and Phosphorylated Tau that Precede a Positive Tau PET in Alzheimer's Disease,” Science Advances 6, eaaz2387 (2020), which are hereby incorporated by reference their entireties). In a particular embodiment, antibodies directed against human tau phosphorylated at threonine at residue 217 can be used to measure the tau load/burden in a subject for the purposes of the present disclosure (see International Patent Application Publication No. WO 2020/242963, which is incorporated by reference in its entirety). The present disclosure includes, in some embodiments, the use of anti-tau antibodies disclosed in WO 2020/242963 to measure the tau load/burden in a subject. The anti-tau antibodies disclosed in WO 2020/242963 are directed against isoforms of human tau expressed in the CNS (e.g., recognizing the isoforms expressed in the CNS and not recognizing isoforms of human tau expressed exclusively outside the CNS). Such antibodies against isoforms of human tau expressed in the CNS can be used in a method of identifying/selecting a patient as one or more of: (i) having a disease disclosed herein; (ii) at risk for having a disease disclosed herein; (iii) in need of treatment for a disease disclosed herein; or (iv) in need of neurological imaging.

In some embodiments, a subject is positive for amyloid plaques when amyloid is detected in the brain by methods such as, amyloid imaging with radiolabeled PET compounds or using a diagnostic that detects Aβ or a biomarker for Aβ. Exemplary methods that can be used in the present disclosure to measure the brain amyloid load/burden include, e.g., Florbetapir (Carpenter, et al., “The Use of the Exploratory IND in the Evaluation and Development of ¹⁸F-PET Radiopharmaceuticals for Amyloid Imaging in the Brain: A Review of One Company's Experience,” The Quarterly Journal of Nuclear Medicine and Molecular Imaging 53.4:387 (2009), which is hereby incorporated by reference in its entirety); Florbetaben (Syed et al., “[¹⁸F]Florbetaben: A Review in β-Amyloid PET Imaging in Cognitive Impairment,” CNS Drugs 29, 605-613 (2015), which is hereby incorporated by reference in its entirety); and Flutemetamol (Heurling et al., “Imaging β-amyloid Using [¹⁸F] Flutemetamol Positron Emission Tomography: From Dosimetry to Clinical Diagnosis,” European Journal of Nuclear Medicine and Molecular Imaging 43.2: 362-373 (2016), which is hereby incorporated by reference in its entirety).

F18-florbetapir can provide a qualitative and quantitative measurement of brain plaque load in patients, including patients with prodromal AD or mild AD dementia. For example, the absence of significant F18-florbetapir signal on a visual read indicates patients clinically manifesting cognitive impairment have sparse to no amyloid plaques. As such, F18-florbetapir also provides a confirmation of amyloid pathology (see, e.g., Clark, et al., “Use of Florbetapir-PET for Imaging β-amyloid Pathology,” JAMA 305.3: 275-283 (2011), which is hereby incorporated by reference in its entirety). F18-florbetapir PET also provides quantitative assessment of fibrillar amyloid plaque in the brain and, in some embodiments, can be used to assess amyloid plaque reductions from the brain by antibodies of the present disclosure. The F18-florbetapir methods can also be automated (see, e.g., Joshi, et al., “A Semiautomated Method for Quantification of F 18 Florbetapir PET Images,” J. Nuclear Medicine 56.11: 1736-1741 (2015), which is hereby incorporated by reference in its entirety).

Amyloid imaging with radiolabeled PET compounds can also be used to determine if Aβ deposit in the brain of a human patient is reduced or increased (e.g., to calculate the percentage reduction in Aβ deposit post treatment or to assess the progression of AD). A person of skill in the art can correlate the standardized uptake value ratio (SUVr) values obtained from amyloid imaging (with radiolabeled PET compounds) to calculate the % reduction in Aβ deposit in the brain of the patient before and after treatment. The SUVr values can be converted to standardized centiloid units, where 100 is average for AD and 0 is average for young controls, allowing comparability amongst amyloid PET tracers, and calculation of reduction according to centiloid units (Klunk et al., “The Centiloid Project: Standardizing Quantitative Amyloid Plaque Estimation by PET,” Alzheimer's & Dementia 11.1: 1-15 (2015) and Navitsky et al., “Standardization of Amyloid Quantitation with Florbetapir Standardized Uptake Value Ratios to the Centiloid Scale,” Alzheimer's & Dementia 14.12: 1565-1571 (2018), which are hereby incorporated by reference in their entireties). In some embodiments, the change in brain amyloid plaque deposition from baseline is measured by F18-florbetapir PET scan.

Cerebrospinal fluid or plasma-based analysis of β-amyloid can also be used to measure the amyloid load/burden for the purposes of the present disclosure. For example, Aβ42 can be used to measure brain amyloid (Palmqvist, S. et al., “Accuracy of Brain Amyloid Detection in Clinical Practice Using Cerebrospinal Fluid Beta-amyloid 42: a Cross-validation Study Against Amyloid Positron Emission Tomography. JAMA Neurol 71, 1282-1289 (2014), which is hereby incorporated by reference in its entirety). In some embodiments, the ratio of Aβ42/Aβ40 or Aβ42/Aβ38 can be used as a biomarker for amyloid beta (Janelidze et al., “CSF Abeta42/Abeta40 and Abeta42/Abeta38 Ratios: Better Diagnostic Markers of Alzheimer Disease,” Ann Clin Transl Neurol 3, 154-165 (2016), which is hereby incorporated by reference in its entirety).

In some embodiments, deposited brain amyloid plaque or Aβ in CSF or plasma can be used to stratify subjects into groups and to identify which group of subjects is responsive to treatment/prevention of a disease (as described herein) using the antibodies, the dosing regimen, or the methods described herein.

As used herein, “anti-N3pGlu Aβ antibody,” “anti-N3pG antibody,” or “anti-N3pE antibody,” used interchangeably, refer to an antibody that binds preferentially to N3pGlu Aβ over Aβ1-40 or Aβ1-42. One of ordinary skill in the art will appreciate and recognize that “anti-N3pGlu Aβ antibody”, and several specific antibodies, including, “hE8L”, “B12L” and “R17L” are identified and disclosed (along with methods for making and using such antibodies) in U.S. Pat. No. 8,679,498 B2 (which is hereby incorporated by reference in its entirety). See, for example, Table 1 of U.S. Pat. No. 8,679,498 B2. Each of the antibodies disclosed in U.S. Pat. No. 8,679,498 B2, including “hE8L”, “B12L” and “R17L” antibodies, may be used as the anti-N3pGlu Aβ antibody of the present disclosure or in place of the anti-N3pGlu Aβ antibodies described in various aspects of the present disclosure. Other representative species of an anti-N3pGlu Aβ antibody include, but are not limited to, antibodies disclosed U.S. Pat. Nos. 8,961,972; 10,647,759; 9,944,696; WO 2010/009987A2; WO 2011/151076A2; WO 2012/136552A1 and equivalents thereto, e.g., under 35 U.S.C 112(f).

One of ordinary skill in the art will appreciate and recognize that “anti-N3pGlu Aβ antibody”, and several specific antibodies are identified and disclosed (along with methods for making and using such antibodies) in U.S. Pat. No. 8,961,972 (which is hereby incorporated by reference in its entirety); U.S. Pat. No. 10,647,759 (which is hereby incorporated by reference in its entirety); and U.S. Pat. No. 9,944,696 (which is hereby incorporated by reference in its entirety). Any of the anti-N3pGlu Aβ antibodies disclosed in the U.S. Pat. Nos. 8,961,972; 9,944,696; and 10,647,759 may be used as the anti-N3pGlu Aβ antibody of the present disclosure or in place of the anti-N3pGlu Aβ antibodies described in various aspects of the present disclosure.

One of ordinary skill in the art will appreciate and recognize that “anti-N3pGlu Aβ antibody”, and several specific antibodies, including, “Antibody VI”, “Antibody VII”, “Antibody VIII”, and “Antibody IX” are identified and disclosed (along with methods for making and using such antibodies) in WO2010/009987A2 (which is hereby incorporated by reference in its entirety). Each of these four antibodies (e.g., “Antibody VI”, “Antibody VII”, “Antibody VIII”, and “Antibody IX”) may be used as the anti-N3pGlu Aβ antibody of the present disclosure or in place of the anti-N3pGlu Aβ antibodies described in various aspects of the present disclosure.

One of ordinary skill in the art will appreciate and recognize that “anti-N3pGlu Aβ antibody”, and several specific antibodies, including, “Antibody X” and “Antibody XI” are identified and disclosed (along with methods for making and using such antibodies) in WO 2011/151076A2 (which is hereby incorporated by reference in its entirety). Each of these two antibodies (e.g., “Antibody X” and “Antibody XI”) may be used as the anti-N3pGlu Aβ antibody of the present disclosure or in place of the anti-N3pGlu Aβ antibodies described in various aspects of the present disclosure.

One of ordinary skill in the art will appreciate and recognize that “anti-N3pGlu Aβ antibody”, and several specific antibodies, including, “Antibody XII” and “Antibody XIII” are identified and disclosed (along with methods for making and using said antibodies) in WO 2012/136552A1 (which is hereby incorporated by reference in its entirety). Each of these two antibodies (e.g., “Antibody XII” and “Antibody XIII”) may be used as the anti-N3pGlu Aβ antibody of the present disclosure or in place of the anti-N3pGlu Aβ antibodies described in various aspects of the present disclosure.

As used herein, an “antibody” is an immunoglobulin molecule comprising two HC and two LC interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present disclosure is based on the following: Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)). Following the above method, the CDRs of the antibodies of the present disclosure were determined.

The antibodies of the present disclosure are monoclonal antibodies (“mAbs”). Monoclonal antibodies can be produced, for example, by hybridoma technologies, recombinant technologies, phage display technologies, synthetic technologies, e.g., CDR-grafting, or combinations of such or other technologies known in the art. The monoclonal antibodies of the present disclosure are human or humanized. Humanized antibodies can be engineered to contain one or more human framework regions (or substantially human framework regions) surrounding CDRs derived from a non-human antibody. Human framework germline sequences can be obtained from ImunoGeneTics (INGT) via their website, http://imgt.cines.fr, or from The Immunoglobulin FactsBook by Marie-Paule Lefranc and Gerard Lefranc, Academic 25 Press, 2001, ISBN 012441351. Techniques for generating human or humanized antibodies are well known in the art. In another embodiment of the present disclosure, the antibody, or the nucleic acid encoding the same, is provided in isolated form. As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

The anti-N3pGlu Aβ antibody of the present disclosure is administered as a pharmaceutical composition. The pharmaceutical composition comprising an antibody of the present disclosure can be administered to a subject at risk for, or exhibiting, diseases or disorders as described herein by parental routes (e.g., subcutaneous, intravenous, intraperitoneal, intramuscular). Subcutaneous and intravenous routes are preferred. In some embodiment, the anti-N3pGlu Aβ antibody is administered by intravenous infusion.

The terms “treatment,” “treating” or “to treat” and the like include restraining, slowing, or stopping the progression or severity of an existing symptom, condition, disease, or disorder in a subject. The term “subject” refers to a human.

The term “prevention” means prophylactic administration of the antibody of the present disclosure to an asymptomatic subject or a subject with pre-clinical Alzheimer's disease to prevent onset or progression of the disease.

The terms “disease characterized by deposition of Aβ” or a “disease characterized by Aβ plaques” are used interchangeably and refer to a disease that is pathologically characterized by Aβ plaques in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes brain plaque imaging, mental or cognitive assessment (e.g., Clinical Dementia Rating—summary of boxes (CDR-SB), Mini-Mental State Exam (MMSE) or Alzheimer's Disease Assessment Scale-Cognitive (ADAS-Cog)) or functional assessment (e.g., Alzheimer's Disease Cooperative Study-Activities of Daily Living (ADCS-ADL). The cognitive and functional assessment can be used to determine changes in a patient's cognition (e.g., cognitive decline) and function (e.g., functional decline). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease, and severe Alzheimer's disease. The term “pre-clinical Alzheimer's disease” is a stage that precedes clinical Alzheimer's disease, where measurable changes in biomarkers (such as CSF Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable. Pre-clinical Alzheimer's disease also includes pre-symptomatic autosomal dominant carriers, as well as patients with higher risk for developing AD by virtue of carrying one or two APOE4 alleles.

A reduction or slowing of cognitive decline can be measured by cognitive assessments such as Clinical Dementia Rating—summary of boxes, Mini-Mental State Exam or Alzheimer's Disease Assessment Scale-Cognitive. A reduction or slowing of functional decline can be measured by functional assessments such as ADCS-ADL.

As used herein, “mg/kg” means an amount, in milligrams, of antibody or drug administered to a subject based on his or her bodyweight in kilograms. A dose is given at one time. For example, a 10 mg/kg dose of antibody for a subject weighing 70 kg would be a single 700 mg dose of antibody given in a single administration. Similarly, a 20 mg/kg dose of antibody for a subject weighing 70 kg would be a 1400 mg dose of antibody given at a single administration.

In some embodiments, each dose of the anti-N3pGlu Aβ antibody is administered to the subject intravenously at a concentration of about 4 mg/mL to about 10 mg/mL over at least 30 minutes. In some embodiments, a 700 mg dose of the anti-N3pGlu Aβ antibody is reconstituted to make 40 mL of reconstituted solution, the reconstituted solution is further diluted to reach an antibody concentration of about 4 mg/mL to about 10 mg/mL, and the diluted solution is administered to the subject intravenously over a duration of 30 minutes. In some embodiments, a 1400 mg dose of the anti-N3pGlu Aβ antibody is reconstituted to make 80 mL of reconstituted solution, the reconstituted solution is further diluted to reach an antibody concentration of about 4 mg/mL to about 10 mg/mL, and the diluted solution is administered to the subject intravenously over a duration of 30 minutes.

As used herein, a human subject has “very low tau” burden if the tau burden is less than 1.10 SUVr (<1.10 SUVr) using ¹⁸F-flortaucipir based quantitative analysis where quantitative analysis refers to calculation of SUVr and SUVr represents counts within a specific target region of interest in the brain (multiblock barycentric discriminant analysis or MUBADA, see Devous et al, “Test-Retest Reproducibility for the Tau PET Imaging Agent Flortaucipir F18,” J. Nucl. Med 59:937-943 (2018)) when compared with a reference region (parametric estimate of reference signal intensity or PERSI, see, Southekal et al., “Flortaucipir F 18 Quantitation Using Parametric Estimation of Reference Signal Intensity,” J. Nucl. Med 59:944-951 (2018)).

As used herein, a human subject has “very low tau to moderate tau” burden if the tau burden is less than or equal to 1.46 SUVr (i.e., ≤1.46 SUVr) using ¹⁸F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)).

As used herein, a human subject has “low tau to moderate tau” burden if the tau burden is from greater than or equal to 1.10 to less than or equal to 1.46 (i.e., ≥1.10 SUVr to 1.46 SUVr) using ¹⁸F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)). “Low tau to moderate tau” burden can also be referred to as “intermediate” tau burden.

As used herein, a human subject has “high tau” burden if the tau burden is greater than 1.46 SUVr (i.e., >1.46 SUVr) using ¹⁸F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)).

As used herein, early symptomatic Alzheimer's disease encompasses the mild cognitive impairment stage of AD (also known as prodromal AD) and the mild dementia stage of AD. The National Institute on Aging and Alzheimer's Association (NIA-AA) created a framework to help define Alzheimer's disease (see, Jack et al., “NIA-AA Research Framework: Toward a Biological Definition of Alzheimer's Disease,” Alzheimer's & Dementia: The Journal of the Alzheimer's Association 14(4) 535-562 (2018), which is hereby incorporated by reference in its entirety).

As used herein, mild cognitive impairment is defined as cognitive performance below expected range for that individual based on all available information. This may be based on clinical judgment and/or on cognitive test performance. Cognitive performance is usually in the impaired/abnormal range based on population norms, but this is not required as long as the performance is below the range expected for that individual. In addition to evidence of cognitive impairment, evidence of decline in cognitive performance from baseline must also be present. This may be reported by the individual or by an observer or observed by change on longitudinal cognitive testing/behavioral assessments or by a combination of these. In this stage, the individual performs daily life activities independently, but cognitive difficulty may result in detectable but mild functional impact on the more complex activities of daily life, either self-reported or corroborated by a study partner. As used herein, mild dementia is defined as substantial progressive cognitive impairment affecting several domains, and/or neurobehavioral disturbance. This is documented by the individual's report or by observer (e.g., study partner) report or by change on longitudinal cognitive testing. This stage includes clear functional impact on daily life, affecting mainly instrumental activities, and the individual is no longer fully independent/requires occasional assistance with daily life activities. An individual no longer is considered to have mild AD dementia when the AD has worsened to the point of a) extensive functional impact on daily life with impairment in basic activities and b) is no longer independent and requires frequent assistance with daily life activities.

As used herein, the term “about” means up to ±10%.

The terms “subject” and “patient” are used interchangeably in the present disclosure.

The term “first dose” and “low dose” can be used interchangeably in the present disclosure. The term “second dose” and “high dose” can be used interchangeably in the present disclosure.

The phrases “slowing of decline” and “slowing disease progression” are used interchangeably in the present disclosure.

As used herein, “methods of treatment” are equally applicable to use of a composition for treating the diseases or disorders described herein and/or compositions for use and/or uses in the manufacture of a medicaments for treating the diseases or disorders described herein.

The following Examples further illustrate the present disclosure. It should be understood however, that the Examples are set forth by way of illustration and not limitation, and that various modifications may be made by one of ordinary skill in the art.

### EXAMPLES

### Example 1: Expression and Purification of Engineered N3pGlu Aβ Antibodies

Antibodies to N3pGlu Aβ are known in the art. For example, U.S. Pat. Nos. 8,679,498 and 8,961,972 (which are hereby incorporated by reference in their entireties) disclose anti-N3pGlu Aβ antibodies, method of making the antibodies, antibody formulations, and methods of treating diseases, such as, Alzheimer's disease with the antibodies.

An exemplary method of expressing and purifying the anti-N3pGlu Aβ antibodies of the present disclosure follows. An appropriate host cell, such as HEK 293 EBNA or CHO, is either transiently or stably transfected with an expression system for secreting antibodies using an optimal predetermined heavy chain to light chain (HC:LC) vector ratio or a single vector system encoding both HC and LC. Clarified media, into which the antibody has been secreted, is purified using any of many commonly used techniques. For example, the medium may be conveniently applied to a Protein A or G Sepharose FF column that has been equilibrated with a compatible buffer, such as phosphate buffered saline (pH 7.4). The column is washed to remove nonspecific binding components. The bound antibody is eluted, for example, by pH gradient (such as 0.1 M sodium phosphate buffer pH 6.8 to 0.1 M sodium citrate buffer (pH 2.5). Antibody fractions are detected, such as by SDS-PAGE, and are pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, or hydroxyapatite chromatography. The purity of the antibody after these chromatography steps can be greater than 99%. The product may be immediately frozen at −70° C. or may be lyophilized. The amino acid sequences for some of the anti-N3pGlu Aβ antibodies of the present disclosure are provided in the sequence listings.

### Example 2: Assessment of Safety, Tolerability, and Efficacy of Anti-N3pGlu Aβ Antibody

A multicenter, randomized, double-blind, placebo-controlled, Phase 2 clinical study (NCT03367403; clinicaltrials.gov) (also known as TRAILBLZER-ALZ or AACG) was designed to evaluate the safety and efficacy of an N3pGlu Aβ antibody (also referred to herein as donanemab) in AD subjects with early symptomatic AD (i.e., subjects with mild cognitive impairment or mild dementia due to AD). This study assessed, including other things, whether removal of existing amyloid plaques can slow progression of disease as determined by clinical measures and biomarkers of disease pathology and neurodegeneration over up to 72 weeks of treatment.

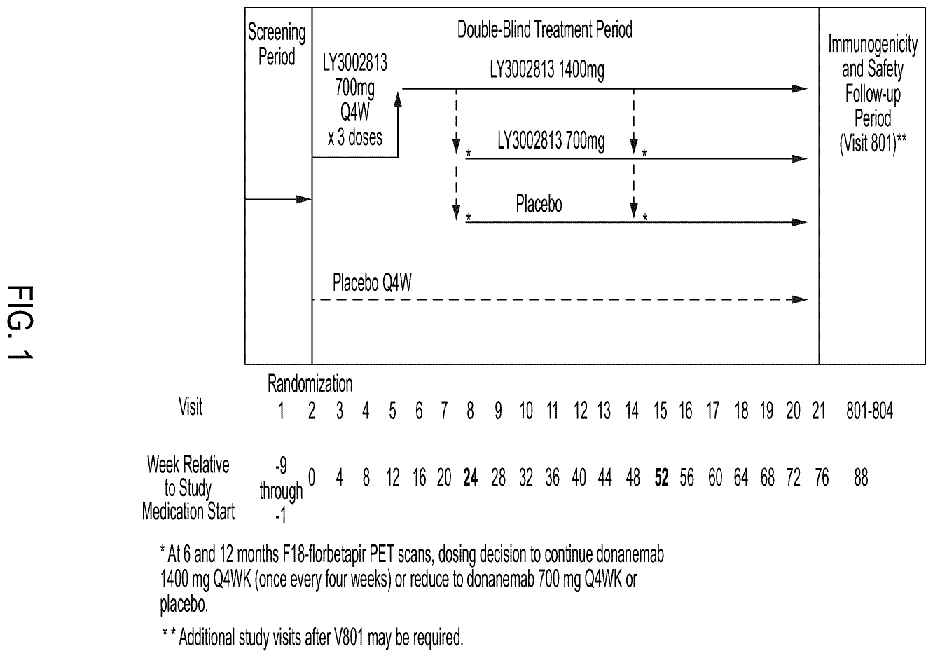

This study was a 133-week study and included a screening period of up to 9 weeks, a treatment period of up to 72 weeks with final evaluations occurring 4 weeks later at Week 76, and a 48-week immunogenicity and a safety follow-up period (see FIG. 1). FIG. 1 illustrates the study design for clinical protocol.

Treatment Arms and Duration: Approximately 1497 patients were screened and approximately 266 were randomized. Patients received the following treatments (dosing) for up to 72 weeks:
Donanemab: intravenous donanemab (700 mg Q4WK for the first 3 doses, then 1400 mg Q4WK) for up to 72 weeks; or
Placebo: intravenous placebo Q4WK for up to 72 weeks.

### Primary and Secondary Endpoints:

The primary endpoint for this study was:
Change in cognition and function as measured by the change in integrated Alzheimer's Disease Rating Scale (iADRS) score from baseline to 18 months.
   The secondary endpoints for this study were:
Change in cognition from baseline to 18 months as measured by: the change in ADAS-Cog₁₃ score, the change in Clinical Dementia Rating Scale Sum of Boxes score (CDR-SB), the change in Mini-Mental State Examination score (MMSE), and the change in Alzheimer's Disease Cooperative Study-instrumental Activities of Daily Living scale (ADCS-iADL) score.
Change in brain amyloid plaque deposition from baseline through 18 months as measured by F18-florbetapir PET scan.
Change in brain tau deposition from baseline to 18 months as measured by F18-flortaucipir PET scan.
Change in volumetric MRI measures from baseline to 18 months.

### Safety Endpoints:

The safety endpoints for this study are:
Standard safety assessments: spontaneously reported adverse events (AEs), clinical laboratory tests, vital sign and body weight measurements, 12-lead electrocardiograms (ECGs), physical, and neurological examinations
MRI (amyloid-related imaging abnormalities [ARIAs] and emergent radiological findings)
Columbia Suicide Severity Rating Scale (C-SSRS)
   Statistical Analysis: All efficacy analyses will follow the intent-to-treat (ITT) principle unless otherwise specified. An ITT analysis is an analysis of data by the groups to which subjects are assigned by random allocation, even if the subject does not take the assigned treatment, does not receive the correct treatment, or otherwise does not follow the protocol. Unless otherwise noted, all pairwise tests of treatment effects was conducted at a 2-sided alpha (a) level of 0.05; 2-sided confidence intervals (CIs) are displayed with a 95% confidence level.
   Efficacy: The primary objective of this study was to test the hypothesis that intravenous infusion of donanemab will slow the cognitive and/or functional decline associated with AD as measured by the composite measure iADRS compared with placebo in patients with early symptomatic AD. The change from baseline score on the iADRS at each scheduled postbaseline visit during the treatment period was analyzed using an MMRM model, which includes the following terms: baseline score, pooled investigator, treatment, visit, treatment-by-visit interaction, baseline-by-visit interaction, concomitant acetylcholine esterase inhibitor (AChEI) and/or memantine use at baseline (yes/no), and age at baseline. The primary time point for treatment comparison was at the end of the double-blind treatment period (Week 76). The treatment group contrast in least-squares mean progression and its associated p-value and 95% CI was calculated for the treatment comparison of donanemab vs. placebo. In addition, Bayesian posterior probability of the active treatment arm being superior to placebo by at least a margin of interest (25% slowing of placebo progression) was calculated.

Change from baseline at each scheduled postbaseline visit during the treatment period in secondary efficacy outcomes, including ADAS-Cog₁₃, ADCS-iADL, CDR-SB, and MMSE, is analyzed using the same MMRM model described for the primary analysis.

Safety: Safety is assessed by summarizing and analyzing adverse events (AEs), laboratory analytes, vital signs, MRI scans, ECGs, immunogenicity during the double-blind treatment period.
Pharmacokinetics Pharmacodynamics: Pharmacokinetic or pharmacodynamic (PK/PD) relationships between plasma donanemab concentration and SUVr, cognitive endpoints, ARIA incidence rate or other markers of PD activity was explored graphically. The relationship between the presence of antibodies to donanemab and PK, PD, safety and/or efficacy may be assessed graphically. If warranted, additional analysis may be explored to evaluate potential interactions for anti-drug antibodies, PD, and other endpoints (PET scan, ARIA-E, etc.). Additional modeling may be performed based on the results of the graphical analyses.
Dosing and Dose Justification: Donanemab (700 mg or 1400 mg) is administered every 4 weeks as an IV infusion of approximately 140 mL over a minimum of 30 minutes. Donanemab doses of 700 mg and 1400 mg administered intravenously once every 4 weeks are selected based on current preclinical pharmacology and toxicology data and clinical PK, PD, and safety data. Prior and ongoing exposures include 0.1 mg/kg, 0.3 mg/kg, 1 mg/kg, 3 mg/kg, 10 mg/kg, 20 mg/kg, and 40 mg/kg in single and/or multi-dose dosing schedules. Data from Study AACC (NCT01837641, clinicaltrials.gov) suggests that PK of donanemab is linear when the dose is not less than 10 mg/kg. Mean half-life is about 9-11 days when dose is ≥10 mg/kg, so minimal accumulation in plasma PK is predicted for 700 mg and 1400 mg Q4 week IV dosing. High levels of F18-florbetapir PET signal reductions are observed with a single dose of 20 mg/kg and are comparable to F18-florbetapir PET reductions seen with a 10 mg/kg Q2 week dosing schedule at 3 months. Based on this as well as decreased patient burden with an every 4 week dosing schedule compared with an every 2 week dosing schedule and comparable safety, 1400 mg Q4 week dosing is selected as the highest dose regimen for robust amyloid plaque lowering. The lowest rate of ARIA-E has been observed with 10 mg/kg monthly dosing. For this reason, a titration schedule (700 mg Q4 week for the first 3 doses, then 1400 mg Q4 week) is proposed to reduce ARIA incidence while allowing patients to achieve high PD effects. In addition, dose reduction rules have been established for incident ARIA-E.
Inclusion Criteria: Patients, including both men and women, 60 to 85 years of age, inclusive, at the time of informed consent were eligible for enrollment in the study. The patients may exhibit gradual and progressive change in memory function reported by patients or study partners (informants) for ≥6 months. In some instances, the patient may have an MMSE score of 20 to 28 (inclusive) at Visit 1 or an acceptable historical F18-flortaucipir PET scan within 6 months prior to Visit 1 that meets central read criterion. The patients may also meet F18-flortaucipir scan (central read) criteria and/or F18-florbetapir scan (central read) criteria.
Exclusion Criteria: Patients are excluded from study enrollment if they meet any of the following criteria: have a Modified Hachinski Ischemia Scale (MHIS; Hachinski et al. 1975) score of ≥4; lack, in the investigator's opinion, of adequate premorbid literacy, adequate vision, or adequate hearing to complete the required psychometric tests; significant neurological disease affecting the central nervous system (CNS), other than AD, that may affect cognition or ability to complete the study, including but not limited to, other dementias, serious infection of the brain, Parkinson's disease, multiple concussions, or epilepsy or recurrent seizures (except febrile childhood seizures); current serious or unstable illnesses including cardiovascular, hepatic, renal, gastroenterological, respiratory, endocrinologic, neurologic (other than AD), psychiatric, immunologic, or hematologic disease and other conditions that, in the investigator's opinion, could interfere with the analyses in this study; or have a life expectancy of <24 months; have a history of cancer within the last 5 years, with the exception of non-metastatic basal and/or squamous cell carcinoma of the skin, in situ cervical cancer, non-progressive prostate cancer, or other cancers with low risk of recurrence or spread; patients with any current primary psychiatric diagnosis other than AD if, in the judgment of the investigator, the psychiatric disorder or symptom is likely to confound interpretation of drug effect, affect cognitive assessment, or affect the patient's ability to complete the study; patients with history of schizophrenia or other chronic psychosis; have a history of long QT syndrome; are clinically judged by the investigator to be at serious risk for suicide as assessed by medical history, examination, or the C-SSRS; history of alcohol or drug use disorder (except tobacco use disorder) within 2 years before the screening visit; have a history of clinically significant multiple or severe drug allergies, or severe post-treatment hypersensitivity reactions (including but not limited to erythema multiforme major, linear immunoglobulin A dermatosis, toxic epidermal necrolysis, and/or exfoliative dermatitis); or have a known positive serologic findings for human immunodeficiency virus (HIV) antibodies. Local laws and regulations may apply to whether testing is required; have any clinically important abnormality at screening, as determined by investigator, in physical or neurological examination, vital signs, ECG, or clinical laboratory test results that could be detrimental to the patient, could compromise the study, or show evidence of other etiologies for dementia; screening MRI which shows evidence of significant abnormality that would suggest another potential etiology for progressive dementia or a clinically significant finding that may impact the patient's ability to safely participate in the study; have any contraindications for MRI, including claustrophobia or the presence of contraindicated metal (ferromagnetic) implants/cardiac pacemaker; have a centrally read MRI demonstrating presence of ARIA-E, >4 cerebral microhemorrhages, more than 1 area of superficial siderosis, any macro hemorrhage or severe white matter disease; an average (ECG in triplicate) corrected QT (QTcF) interval measurement>450 msec (men) or >470 msec (women) at screening (as determined at the investigational site; patients with a past history of Hepatitis B should have HBsAg testing at screening and are excluded if HBsAg is positive; patients with past history of Hepatitis C should have HCV RNA PCR testing at screening and are excluded if HCV RNA PCR is positive; calculated creatinine clearance<30 mL/min (Cockcroft-Gault formula; Cockcroft and Gault 1976) at screening; alanine transaminase (ALT)≥2× the upper limit of normal (ULN) of the performing laboratory, aspartate aminotransferase (AST)≥2×ULN, total bilirubin level (TBL)≥1.5×ULN, or alkaline phosphatase (ALP)≥1.5×ULN at screening; have received treatment with a stable dose of an AChEI and/or memantine for less than 2 months before randomization; changes in concomitant medications that could potentially affect cognition and their dosing should be stable for at least 1 month before screening, and between screening and randomization (does not apply to medications discontinued due to exclusions or with limited duration of use, such as antibiotics); current use of drugs known to significantly prolong the QT interval; have had prior treatment with a passive anti-amyloid immunotherapy<5 half-lives prior to randomization; have received active immunization against Aβ in any other study; have known allergies to donanemab, related compounds, or any components of the formulation; or history of significant atopy; have allergies to either monoclonal antibodies, diphenhydramine, epinephrine, or methylprednisolone; sensitivity to F18-florbetapir or F18-flortaucipir; contraindication to MRI; contraindication to PET; present or planned exposure to ionizing radiation that, in combination with the planned administration of study PET ligands, would result in a cumulative exposure that exceeds local recommended exposure limits.
Dosage Modification for ARIA-E: Donanemab dosage modifications are adjusted for the occurrence of ARIA-E in the following instances depicted in Table A. If a dosage reduction is required, the donanemab dose is reduced to the next lower dose (from 1400 mg to 700 mg or from 700 mg to placebo).

TABLE A
Donanemab Dosage Modifications for First Occurrence of ARIA-E

| ARIA-E | ARIA-E on MRI | | |
|---|---|---|---|
| SYMPTOMS | Mild | Moderate | Severe |
| No symptoms | Continue current | donanemab | Temporary |
| | dosing^{a} | Dose reduction^{a,b} | Discontinuation |
| | | | of donanemab |
| Mild | donanemab | Temporary | Temporary |
| | Dose reduction^{a,b} | Discontinuation | Discontinuation |
| | | of donanemab | of donanemab |
| Moderate | Temporary | Temporary | Temporary |
| | Discontinuation | Discontinuation | Discontinuation |
| | of donanemab | of donanemab | of donanemab |
| Severe | Temporary | Temporary | Temporary |
| | Discontinuation | Discontinuation | Discontinuation |
| | of donanemab | of donanemab | of donanemab |

^{a}Investigator may choose to temporarily discontinue donanemab after discussion with the sponsor.
^{b}If the patient has a second incidence of ARIA-E and has previously been dose reduced or temporarily discontinued from donanemab, then donanemab is permanently discontinued.

All cases of ARIA-E will require unscheduled MRI scans every 4-6 weeks until ARIA-E has resolved.
Discontinuation from Study Treatment: Possible reasons leading to permanent discontinuation of study treatment: Subject Decision (the subject or the subject's designee; for example, legal guardian requests to discontinue investigational product) or discontinuation due to a hepatic event or liver test abnormality. Subjects who are discontinued from investigational product due to a hepatic event or liver test abnormality should have additional hepatic safety data collected via CRF/electronic data entry.

Discontinuation of the investigational product for abnormal liver tests is considered when a subject meets one of the following conditions: alanine aminotransferase (ALT) or aspartate aminotransferase (AST)>8× upper limit of normal (ULN); ALT or AST>5×ULN for more than 2 weeks; ALT or AST>3×ULN and total bilirubin level (TBL)>2×ULN or international normalized ratio (INR)>1.5; ALT or AST>3×ULN with the appearance of fatigue, nausea, vomiting, right upper-quadrant pain or tenderness, fever, rash, and/or eosinophilia (>5%); alkaline phosphatase (ALP)>3×ULN; ALP>2.5×ULN and TBL>2×ULN; or ALP>2.5×ULN with the appearance of fatigue, nausea, vomiting, right quadrant pain or tenderness, fever, rash, and/or eosinophilia (>5%).

In addition, subjects are discontinued from the investigational product in the following circumstances:
Treatment with donanemab is permanently discontinued in patients with:
   a second incidence of ARIA-E after a previous dose reduction or temporary discontinuation of donanemab;
   any increase in ARIA-H accompanied by clinically significant symptoms;
   >4 new microhemorrhages, >1 new area of superficial siderosis or significant worsening of pre-existing superficial siderosis, or any macro hemorrhage regardless of symptoms; or
   an ARIA-E event reported as a significant adverse event (SAE), regardless of severity of symptoms or MRI findings.
Treatment with donanemab will also be permanently discontinued in patients with:
   Prolonged acute infusion reaction (i.e., not responsive to medication such as antihistamines, nonsteroidal anti-inflammatory drugs, and/or narcotics and/or brief interruption of infusion); or
   Adverse event or clinically significant laboratory value, ECG result, physical examination finding, MRI finding (such as symptomatic ischemic stroke),
      Temporary Discontinuation from Donanemab Study Treatment Due to ARIA-E

Temporary discontinuation from donanemab treatment is allowed for ARIA-E if the ARIA-E meets the temporary discontinuation criteria shown in Table A. In cases of ARIA-E where the protocol indicates continued dosing or a dose reduction rather than temporary discontinuation, the administration of donanemab may be temporarily discontinued.

Donanemab may be restarted following a first incidence of ARIA-E if, e.g., dosing is temporarily discontinued due to ARIA-E and there is complete resolution of symptoms and radiologic findings within 16 weeks after the temporary drug discontinuation. If ARIA-E symptoms and radiologic findings have not completely resolved within 16 weeks, then the patient is permanently discontinued from donanemab treatment.

Study drug may be restarted at either 700 mg or placebo, double blinded, depending on the original study arm to which the patient is randomized. An unscheduled safety MRI scan is required 4-6 weeks after dose restarts.

Efficacy Assessments: Cognitive and functional testing is administered using an eCOA tablet. The audio voice recordings of the rater's questions and the patient's and study partner's responses will also be collected via the eCOA tablet during administration of the cognitive and functional testing for central monitoring of rater scale administration. Cognitive and functional testing for each patient should be performed at approximately the same time on each day that testing occurs to reduce potential variability. Note that the ADAS-Cog and MMSE must be administered by a different rater than the ADCS-ADL and CDR. These 2 raters should continue doing the same scale with the same patient throughout the study. If possible, each assessment should be performed on a given patient by the same rater at each visit. The principal investigator (PI) has the responsibility of selecting the raters who will administer the instruments at the site if all training requirements have been met by those raters.

When administered, cognitive and functional testing should be performed first, before medical procedures that could be stressful for the patient (e.g., blood draws). Note that some procedures (MRI, F18-flortaucipir PET tau imaging, F18-florbetapir PET amyloid imaging) can be conducted on other days within the visit window.

### Primary Efficacy Assessments:

Integrated Alzheimer's Disease Rating Scale (iADRS; Wessels et al., “A Combined Measure of Cognition and Function for Clinical Trials: The Integrated Alzheimer's Disease Rating Scale (iADRS),” J Prev Alzheimer's Dis. 2(4):227-241 (2015), which is hereby incorporated by reference in its entirety). The iADRS represents a composite that was developed using both a theory-driven approach (incorporating measures of both cognition and function) and a data-mining approach (identifying the most sensitive combination of scales through analysis of data from the Alzheimer's Disease Neuroimaging Initiative). The iADRS is a simple linear combination of scores from 2 well-established, therapeutically sensitive, widely accepted measures in AD, the ADAS-Cog₁₃ and the ADCS-iADL, measuring the core domains of AD. All items of these 2 scales are included without additional weighting of items, yielding face validity and ease of interpretation of the composite relative to its components. The iADRS score is derived from the ADAS-Cog₁₃ and the ADCS-iADL and is the primary efficacy measure. The ADAS-Cog₁₃ and the ADCS-ADL are the actual scales administered to patients.
Secondary Efficacy Assessments: Additional clinical outcome measures should be administered in the same order at every visit, immediately following assessment of the ADAS-Cog₁₃. To minimize missing data, the rater should include each measure orally with the patient or study partner (as designated in instructions), recording responses appropriately. The same study partner should be used as the informant at all visits.
Alzheimer's Disease Assessment Scale Cognitive subscale: The ADAS-Cog₁₃ is a rater-administered instrument that was designed to assess the severity of the dysfunction in the cognitive and noncognitive behaviors characteristic of persons with AD (Rosen et al., “A New Rating Scale for Alzheimer's Disease,” Am J Psychiatry. 141(11):1356-1364 (1984), which is hereby incorporated by reference in its entirety). The ADAS-Cog₁₃ should be administered by the same rater from visit to visit to reduce potential variability. The cognitive subscale of the ADAS, the ADAS-Cog₁₃, consists of 13 items assessing areas of cognitive function most typically impaired in AD: orientation, verbal memory, language, praxis, delayed free recall, digit cancellation, and maze-completion measures (Mohs et al., “Development of Cognitive Instruments for Use in Clinical Trials of Antidementia Drugs: Additions to the Alzheimer's Disease Assessment Scale that Broaden its Scope,” The Alzheimer's Disease Cooperative Study. Alzheimer Dis Assoc Disord. 11(Suppl 2):S13-S21 (1997), which is hereby incorporated by reference in its entirety). The ADAS-Cog₁₃ allows better discrimination of differences among mild patients than the ADAS-Cog11 and is included as a secondary outcome. The ADAS-Cog₁₃ scale ranges from 0 to 85, with higher scores indicating greater disease severity.
Alzheimer's Disease Cooperative Study—Activities of Daily Living Inventory: The ADCS-ADL is a 23-item inventory developed as a rater-administered questionnaire that is to be answered by the patient's study partner (Galasko et al., “An Inventory to Assess Activities of Daily Living for Clinical Trials in Alzheimer's Disease,” The Alzheimer's Disease Cooperative Study. Alzheimer Dis Assoc Disord. 1997; 11(Suppl 2):S33-S39; Galasko et al., “Galantamine Maintains Ability to Perform Activities of Daily Living in Patients with Alzheimer's Disease,” J Am Geriat Soc. 52(7):1070-1076 (2004), which are hereby incorporated by reference in their entireties). The ADCS-ADL should be administered by the same rater from visit to visit to reduce potential variability. The ADCS-ADL subset of items (items 7 to 23) for instrumental activities of daily living (ADCS-iADL) is used as a secondary efficacy measure. The focus in the early symptomatic AD population is on the instrumental activities of daily living (iADLs) rather than the basic activities of daily living (bADLs), which are thought to be affected in more severe stages of the disease. The range for the iADL score is 0 to 56, with lower scores indicating greater disease severity. For each of the specific items, the study partner is first asked if the patient attempted the ADL during the past 4 weeks. If the patient did attempt the ADL, the study partner is asked to rate the patient's performance level based on a set of performance descriptions. Scores for each item and the overall score for the tool are calculated. The range for the total ADCS-ADL score is 0 to 78, with higher scores indicating greater level of impairment. Separate scores for the bADLs (0 to22) are also be computed.
Clinical Dementia Rating Scale: The CDR is a semi-structured interview performed with the patient and study partner (informant) that provides an index of global functioning (Berg et al., “Mild Senile Dementia of the Alzheimer's Type. 4. Evaluation of Intervention,” Ann Neurol. 31(3):242-249 (1992), which is hereby incorporated by reference in its entirety). The CDR should be administered by the same rater from visit to visit to reduce potential variability. The informant is queried about the patient's memory, orientation, judgment, and problem solving, community affairs, home and hobbies, and personal care. The patient's memory, orientation, judgment, and problem-solving ability are assessed. Higher scores indicate greater disease severity. By assigning a severity score for each of the 6 domains, a total score known as sum of boxes is obtained-hence the abbreviation, CDR-SB. The range for CDR-SB is from 0 to 18, higher values indicating greater impairment
Mini-Mental State Examination: The MMSE is a brief instrument used to assess cognitive function in patients (Folstein et al., “Mini-Mental State”. A Practical Method for Grading the Cognitive State of Patients for the Clinician,” J Psychiatr Res. 12(13):189-198 (1975), which is hereby incorporated by reference in its entirety). The MMSE should be administered by the same rater from visit to visit to reduce potential variability. The instrument is divided into 2 sections. The first section measures orientation, memory, and attention. The maximum score for the first section is 21. The second section tests the ability of the patient to name objects, follow verbal and written commands, write a sentence, and copy figures. The maximum score for the second section is 9. The range for the total MMSE score is 0 to 30, with lower scores indicating great level of impairment.
Biomarker Efficacy Measures (Double-Blind Period) F18-florbetapir PET scan: Change in amyloid burden (as assessed by F18-florbetapir PET signal) is compared in donanemab- and placebo-treated patients for those patients who undergo F18-florbetapir PET scans at baseline, Week 52 [Visit 15], and Week 76 [Visit 21] or an early discontinuation visit (ED).
F18-flortaucipir PET scan: Change in tau burden (as assessed by F18-flortaucipir PET signal) is compared in donanemab- and placebo-treated patients for those patients who undergo both baseline and endpoint (Visit 21 [Week 76] or ED) F18-flortaucipir scans.
Volumetric MRI: Magnetic resonance imaging of the brain may be performed during visits 2-14. Donanemab- and placebo-treatment effects on volumetric MRI is assessed and compared to evaluate the loss of brain volume that occurs in AD patients.
Clearance of Amyloid Plaques: Clearance of amyloid plaques (as assessed by F18-florbetapir PET signal) is compared in donanemab- and placebo-treated patients for those patients who undergo baseline, Visit 8 (Week 24), Visit 15 (Week 52) and endpoint Visit 21 (Week 76), or ED F18-florbetapir PET scans.
Accumulation of Tau Deposits: Extent of accumulation of tau paired helical filaments (PHF) plaques (as assessed by F18-flortaucipir PET signal) is compared in donanemab- and placebo-treated patients for those patients who undergo baseline and endpoint Visit 21 (Week 76), or ED F18-flortaucipir PET scans.
Biomarkers: Biomarker research is performed to address questions of relevance to drug disposition, target engagement, PD, mechanism of action, variability of patient response (including safety), and clinical outcome. Sample collection is incorporated into clinical studies to enable examination of these questions through measurement of biomolecules including deoxyribonucleic acid (DNA), ribonucleic acid (RNA), proteins, lipids, and other cellular elements. Serum, plasma, and whole blood RNA samples for biomarker research are collected during visits 2-14, where local regulations allow.

### Example 3: Results from Safety, Tolerability, and Efficacy Study

This example provides the results obtained from safety, adverse events, and efficacy of donanemab in participants with early symptomatic AD. Enrolment was based on florbetapir and flortaucipir Positron Emission Tomography (PET) scan demonstrating tau and amyloid plaque pathology, respectively. Participants received either intravenous placebo or donanemab (700 mg for doses 1-3 and 1400 mg thereafter) every 4 weeks for up to 72 weeks. The primary outcome measure was change from baseline in the integrated AD Rating Scale (iADRS; range 0 to 144, lower indicating greater cognitive deficit and impairment of activities of daily living) at 76 weeks. Secondary outcome measures included Clinical Dementia Rating Scale Sum of Boxes (CDR-SB; range 0 to 18, higher indicating greater impairment), AD Assessment Scale-Cognitive (ADAS-Cog₁₃; range 0 to 85, higher indicating greater disease severity), AD Cooperative Study-instrumental Activities of Daily Living (ADCS-iADL; range 0 to 59, lower indicating greater impairment), Mini-Mental State Examination (MMSE; range 0 to 30, lower indicating greater impairment), amyloid and tau burden as assessed by florbetapir and F18-flortaucipir PET, respectively, and volumetric magnetic resonance imaging MRI (vMRI).

Patient population and study design: This study (TRAILBLAZER-ALZ) is a multi-center, randomized, double-blind, placebo-controlled study assessing the safety, adverse events, and efficacy of donanemab in participants with early symptomatic AD (the combination of prodromal AD, the symptomatic pre-dementia phase of AD where MCI is apparent [MCI-AD], and mild AD dementia [symptoms are sufficiently severe to meet dementia and AD diagnostic criteria]) aged 60-85 years (Dubois et al., “Research Criteria for the Diagnosis of Alzheimer's Disease: Revising the NINCDS-ADRDA Criteria,” The Lancet Neurology 6:734-46 (2007), which is hereby incorporated by reference in its entirety). Screening procedures included the Mini-Mental State Examination (MMSE; range 0 to 30, lower indicating greater impairment, Folstein et al., “Mini-mental state. A Practical Method for Grading the Cognitive State of Patients for the Clinician,” J Psychiatr. Res. 12:189-98 (1975), which is hereby incorporated by reference in its entirety)), F18-flortaucipir PET scan, magnetic resonance imaging (MRI), and F18-florbetapir PET scan. The flortaucipir and F18-florbetapir PET scans were reviewed by a centralized PET imaging facility for assessment of patient's eligibility. All eligible patients were required to have evidence of pathologic tau on PET scan and quantitative tau levels below a specific upper threshold. The latter criterion addressed the concern that anti-amyloid treatments would have limited efficacy in advanced disease, as indicated by the presence of extensive tau pathology. Tau images were quantitatively evaluated to estimate an SUVr (standardized uptake value ratio) by published methods (Pontecorvo et al., “A Multicentre Longitudinal Study of Flortaucipir (¹⁸F) in Normal Ageing, Mild Cognitive Impairment and Alzheimer's Disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and visually evaluated (Fleisher et al., “Positron Emission Tomography Imaging With F18-flortaucipir and Postmortem Assessment of Alzheimer Disease Neuropathologic Changes,” JAMA Neurology 77:829-39 (2020), which is hereby incorporated by reference in its entirety) for whether they had an AD pattern.

Any image with an SUVr>1.46 was excluded as having high tau. For those images not excluded as having high tau, images with SUVr values<1.10 or images that were visually read as having a negative AD pattern were excluded for having inadequate tau levels with the exception that if the image was visually read as having an advanced tau AD pattern but SUVr value was <1.10, the case would still be included. Except for MRI, each patient was required to meet all other Visit 1 eligibility criteria prior to a screening F18-florbetapir PET scan.

Participants who met entry criteria were randomized 1:1 to receive either intravenous (IV) donanemab every 4 weeks (700 mg for the first 3 doses, 1400 mg thereafter) or IV placebo every 4 weeks, for up to 72 weeks. For between-group comparability for site factors, participant randomization was stratified by investigative site. There was no stratification by entry criteria. In participants treated with donanemab, the dose was downtitrated to 700 mg if amyloid removal in centiloids (CL) measured by florbetapir scan (at 24 and 52 weeks) was ≥11 and <25 or switched to placebo if <11 at any one measure or ≥11 and <25 for two consecutive scans. If amyloid-related imaging abnormalities-edema/effusions (ARIA-E; signal hyperintensities on an MRI in fluid-attenuated inversion recovery imaging sequences, due to parenchymal fluid accumulation or sulcal fluid effusion; Sperling et al., “Amyloid-related Imaging Abnormalities in Amyloid-Modifying Therapeutic Trials: Recommendations from the Alzheimer's Association Research Roundtable Workgroup,” Alzheimer's & Dementia 7:367-85 (2011), which is hereby incorporated by reference in its entirety) occurred during the up-titration with the first three doses of 700 mg, the dose was not escalated. Final endpoint measures and safety assessments were performed at Week 76, 4 weeks after the last infusion.

Clinical and biomarker outcome measures: The primary outcome measure was the change from baseline to 76 weeks in the change on the iADRS (range 0 to 144, lower indicating greater cognitive deficit and impairment of daily living), compared with placebo. The iADRS is a linear combination of its individual components, the AD Assessment Scale-Cognitive (ADAS-Cog₁₃; range 0 to 85, higher indicating greater disease severity; Mohs et al., “Development of Cognitive Instruments for use in Clinical Trials of Antidementia Drugs: Additions to the Alzheimer's Disease Assessment Scale that Broaden its Scope. The Alzheimer's Disease Cooperative Study,” Alzheimer Dis Assoc Disord 11 Suppl 2:S13-21 (1997), which is hereby incorporated by reference in its entirety) and the AD Cooperative Study-instrumental Activities of Daily Living (ADCS-iADL; range 0 to 59, lower indicating greater impairment; Galasko et al., “An Inventory to Assess Activities of Daily Living for Clinical Trials in Alzheimer's disease,” Alzheimer Disease and Associated Disorders 11:S33-S9 (1997) and Galasko et al., “Galantamine Maintains Ability to Perform Activities of Daily Living in Patients with Alzheimer's Disease,” Journal of the American Geriatrics Society 52:1070-6 (2004), which are hereby incorporated by reference in their entirety).

The iADRS was developed using the theoretical construct of aiming to measure core disease processes and clinical trial data was used to identify items/scales that performed best for that construct. All items of the ADAS-Cog₁₃ total score and ADCS-iADL score are included without weighting of items, yielding face validity and ease of interpretation of both the composite and its components. The iADRS allows for an overall measure of AD impairment (total score) as well as individual subscores (cognition and function). Validation of iADRS has been established and statistical properties of the composite performance have been described.

Methodologies for the secondary outcome measures, Clinical Dementia Rating Scale Sum of Boxes (CDR-SB; range 0 to 18, higher indicating greater impairment; Morris, “The Clinical Dementia Rating (CDR),” Current Version and Scoring Rules 43:2412-a (1993), which is hereby incorporated by reference in its entirety), ADAS-Cog₁₃, ADCS-iADL, MMSE, amyloid and tau burden as assessed by F18-florbetapir and F18-flortaucipir PET respectively, and volumetric MRI are detailed in the clinical protocol. Assessment of global tau load was carried out using a Tau^{IQ} algorithm (Whittington et al., “TauIQ-A Canonical Image Based Algorithm to Quantify Tau PET Scans,” J. of Nuclear Medicine (2021), which is hereby incorporated by reference in its entirety) accounting for the spatiotemporal distribution of tau.

Determination of sample size and statistical analysis: Enrollment of 250 participants randomized 1:1 to two treatment arms, with 200 participants expected to complete treatment, was determined to provide approximately 84% power to demonstrate that the active treatment arm had a ≥0.6 posterior probability of slowing iADRS progression over placebo by at least 25%. The assumption for power calculation was mean progression levels in the placebo and donanemab arms of approximately 12 and 6 points (50% slowing) over 18 months, respectively, with common standard deviation of 17. Efficacy analyses were conducted based on a modified intention-to-treat principle (unless otherwise specified) where participants had a baseline and at least one post-baseline iADRS measurement. Unless otherwise noted, all pairwise tests of treatment effects were conducted at a 2-sided alpha level of 0.05.

Baseline characteristics were summarized by treatment group and overall, with descriptive statistics for continuous and categorical measures. The primary outcome was analyzed with the use of a mixed-model repeated-measures (MMRM) analysis, with the change from baseline in the iADRS score at each scheduled postbaseline timepoint as the dependent variable. The model for the fixed effects included the following terms: baseline score, investigator, treatment, visit, treatment-by-visit interaction, baseline-by-visit interaction, concomitant acetylcholinesterase inhibitors and/or memantine use at baseline (yes/no), and age at baseline. Visit was considered a categorical variable. Secondary efficacy outcomes were assessed using a MRM analysis. Bretz's graphical approach (Bretz, et. al., “A Graphical Approach to Sequentially Rejective Multiple Test Procedures,” Statistics in Medicine, 28(4):586-604 (2009), which is hereby incorporated by reference in its entirety) was used to provide control of the study-wise type I error rate for the primary and key secondary hypotheses at alpha level of 0.05. Assuming that the primary analysis was significant, the MMRM analyses described for the primary analysis was conducted on the CDR-SB, ADAS-Cog₁₃, ADCS-iADL and MMSE scores and significance determined based on a multiplicity graph of hypotheses. Longitudinal clinical outcomes are provided with point estimates and error bars. For post-baseline categorical data, Fisher's exact test was used for treatment-group comparisons. For post-baseline continuous data collected at endpoint, analysis of covariance (ANCOVA), with independent factors for treatment and age, was used. Each principal site investigator was responsible for selecting raters, who met training requirements, to administer the instruments at the site. Raters were blinded to treatment assignment.

The Bayesian Disease Progression Model (DPM) was used to assess the rate of decline of the iADRS between the donanemab group and the placebo group across the 76 weeks of the study. The model assumes a proportional treatment effect relative to placebo and includes diffuse priors. A similar model was previously used, with the exception that in the current model the prior distributions on the parameters representing the placebo decline were not forced to be monotonic. The analysis generated a posterior probability distribution of the disease progression ratio (DPR), defined as the proportional decline of the donanemab arm relative to placebo. A DPR of less than 1 favors donanemab. The 95% credible intervals and the posterior mean of the disease progression ratios are presented. The posterior probability of the active treatment arm slowing the disease progression by at least 25% relative to placebo was pre-specified and calculated from the DPM. The DPM model was used to assess the rate of decline of the CDR-SB, ADAS-Cog₁₃, ADCS-iADL and MMSE. DPM models were not included as part of pre-specified multiplicity testing strategy for secondary endpoints.

Safety parameters (AEs, laboratory analytes, vital signs, electrocardiograms, and MRIs) were summarized using descriptive statistics for continuous variables and frequencies along with percentages for categorical variables during the treatment period.

A likelihood-based mixed effects model for repeated measures was used to handle missing data for the MMRM model. The model parameters were simultaneously estimated using restricted likelihood estimation incorporating all observed data. Estimates have been shown to be unbiased when the missing data are missing at random and when there is ignorable non-random missing data. Repeated measures analyses only used data from visits where the data was scheduled to be collected. When participants discontinued from the study early, there may have been efficacy or safety data measurements at visits where the variables were not scheduled to be collected. This data was used in all other analyses.

Population and Baseline Characteristics: Baseline demographics for the placebo and donanemab monotherapy groups, respectively, for mean age were 75.4 and 75.0 years, for female sex were 51.6% and 51.9%, for white race were 96.0% and 93.1%, and for APOE4 carrier were 74.2% and 72.5% (Table B).

TABLE B
Characteristics of Trial Participants at Baseline

| | | | Donanemab | | | |
|---|---|---|---|---|---|---|
| | Placebo | | Monotherapy | | Total † | |
| | (N = 126) | | (N = 131) | | (N = 272) | |
| Demographic | | | | | | |
| Female sex, n (%) | 65 | (51.6) | 68 | (51.9) | 145 | (53.3) |
| Mean age, years (SD) | 75.4 | (5.4) | 75.0 | (5.6) | 75.2 | (5.5) |
| Race, n (%) | | | | | | |
| Asian | 2 | (1.6) | 1 | (0.8) | 3 | (1.1) |
| Black or African | 3 | (2.4) | 5 | (3.8) | 8 | (2.9) |
| American | | | | | | |
| White | 121 | (96.0) | 122 | (93.1) | 258 | (94.9) |
| Other* | 0 | (0) | 3 | (2.3) | 3 | (1.1) |
| Ethnicity, Hispanic/Latino ^{#}, n | 3 | (2.4) | 5 | (3.8) | 9 | (3.3) |
| (%) | | | | | | |
| Education, ≥13 years, n (%) | 102 | (81.0) | 97 | (74.0) | 209 | (76.8) |
| APOE 4 carrier, n/N (%) | 92/124 | (74.2) | 95/131 | (72.5) | 197/270 | (73.0) |
| E2/E3, n (%) | 1 | (0.8) | 1 | (0.8) | 2 | (0.7) |
| E2/E4, n (%) | 2 | (1.6) | 2 | (1.5) | 4 | (1.5) |
| E3/E3, n (%) | 31 | (25.0) | 35 | (26.7) | 71 | (26.3) |
| E3/E4, n (%) | 62 | (50.0) | 68 | (51.9) | 137 | (50.7) |
| E4/E4, n (%) | 28 | (22.6) | 25 | (19.1) | 56 | (20.7) |
| AChEI use, n (%) | 74 | (58.7) | 78 | (59.5) | 162 | (59.6) |
| Scale, Mean (SD), range | | | | | | |

| | | | |
|---|---|---|---|
| iADRS | 105.9 (13.2), | 106.2 (13.0) ^{a}, | 106.2 (13.0) ^{b}, |
| | 67.0-139.0 | 60.0-130.0 | 60.0-139.0 |
| CDR-SB | 3.4 (1.7), | 3.6 (2.1), | 3.5 (1.9), |
| | 0.5-8.0 | 0.5-11.0 | 0.5-11.0 |
| ADAS-Cog₁₃ | 27.5 (7.6), | 27.6 (7.7), | 27.6 (7.6), |
| | 5.0-47.0 | 10.0-51.0 | 5.0-51.0 |
| ADCS-ADL | 67.0 (8.1), | 67.4 (8.6) ^{a}, | 67.3 (8.2) ^{b}, |
| | 40.0-78.0 | 28.0-78.0 | 28.0-78.0 |
| ADCS-iADL | 48.4 (7.5), | 48.9 (7.6) ^{a}, | 48.8 (7.5) ^{b}, |
| | 24.0-59.0 | 21.0-59.0 | 21.0-59.0 |
| MMSE | 23.7 (2.9) ^{c}, | 23.6 (3.1) ^{d}, | 23.5 (3.1) ^{e}, |
| | 16.0-29.0 | 14.0-29.0 | 13.0-30.0 |
| Amyloid PET Centiloids, | 101.1 (33.3), | 107.6 (36.0), | 104.2 (34.8), |
| Mean (SD), range | 38.7-225.2 | 41.0-251.4 | 38.7-251.4 |
| Flortaucipir PET global tau | 0.46 (0.15) ^{f}, | 0.47 (0.19) ^{g}, | 0.46 (0.17) ^{h}, |
| load, Mean (SD), range | 0.2-0.9 | 0.1-1.2 | 0.1-1.2 |

*Note:
includes Multiple & American Indian or Alaska Native.
† includes participants in the combo group.
^{#} number of participants with non-missing data, used as denominator,
^{a} Donanemab monotherapy N = 130,
^{b} Total N = 271,
^{c} Placebo N = 121,
^{d} Donanemab monotherapy N = 126,
^{e} Total N = 261,
^{f} Placebo N = 124,
^{g} Donanemab monotherapy N = 130,
^{h} Total N = 269.
APOE 4 = Apolipoprotein E allele 4; AChEI = Acetylcholinesterase Inhibitor; ADAS-Cog₁₃ = AD Assessment Scale-Cognitive 13-item Subscale; ADCS-ADL = Alzheimer's Disease Cooperative Study Activities of Daily Living Inventory; ADCS-iADL = Alzheimer's Disease Cooperative Study-instrumental Activities of Daily Living Inventory; iADRS = integrated Alzheimer's Disease Rating Scale; MMSE = Mini-Mental State Examination; CDR-SB = Clinical Dementia Rating Scale Sum of Boxes; PET = Positron Emission Tomography; N/n = Number of participants; SD = Standard Deviation.

At the time of trial initiation, the study consisted of three arms including a combination group of donanemab with BACE 1 inhibitor. This arm was discontinued early in the trial with 15 participants randomized to that group. In the modified intention-to-treat population, of the 1955 participants screened, 126 were randomized to placebo, 131 to donanemab. The mean baseline scores for iADRS were 105.9 for placebo and 106.2 for donanemab; respectively for MMSE were 23.7 and 23.6; CDR-SB were 3.4 and 3.6; F18-flortaucipir PET global tau loads were 0.46 and 0.47; amyloid PET values were 101.1 and 107.6 (Table B).

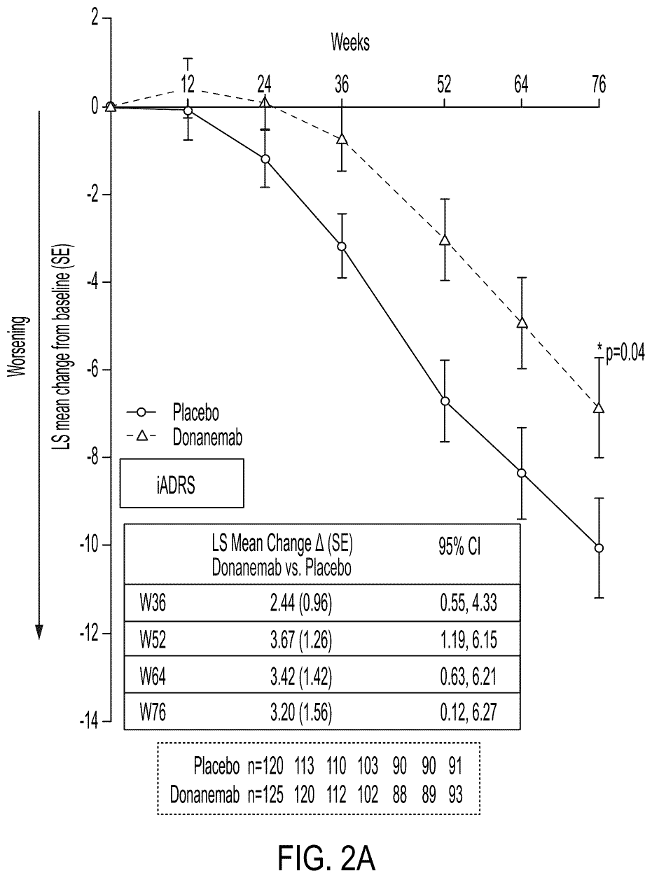

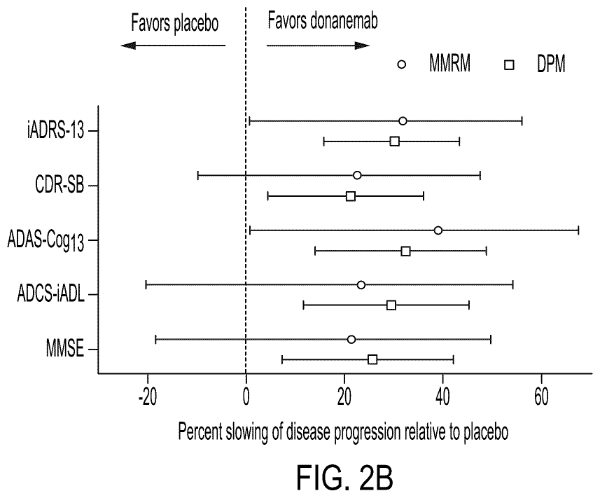

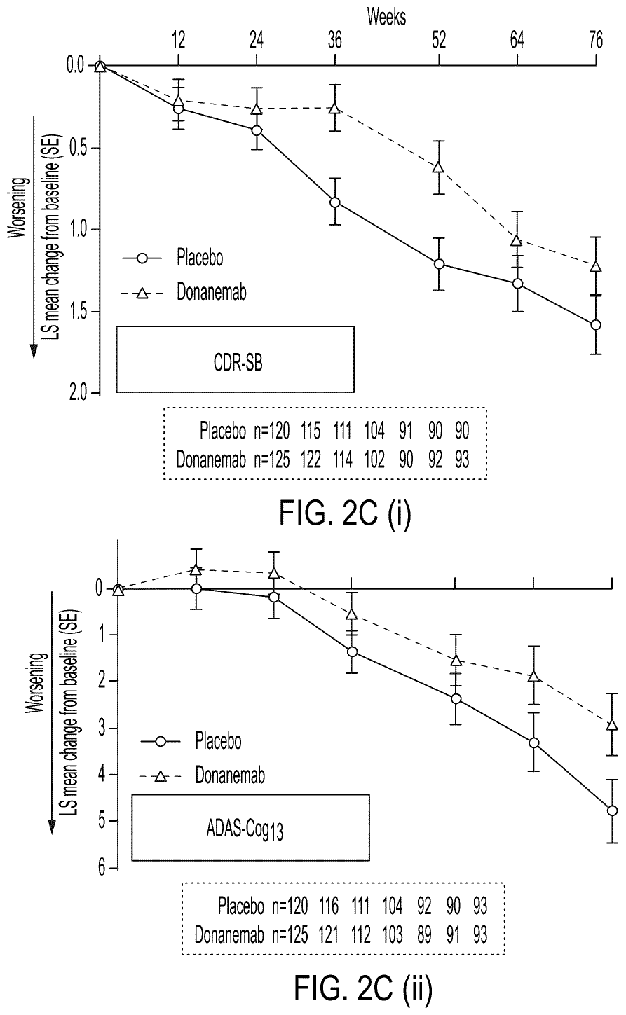

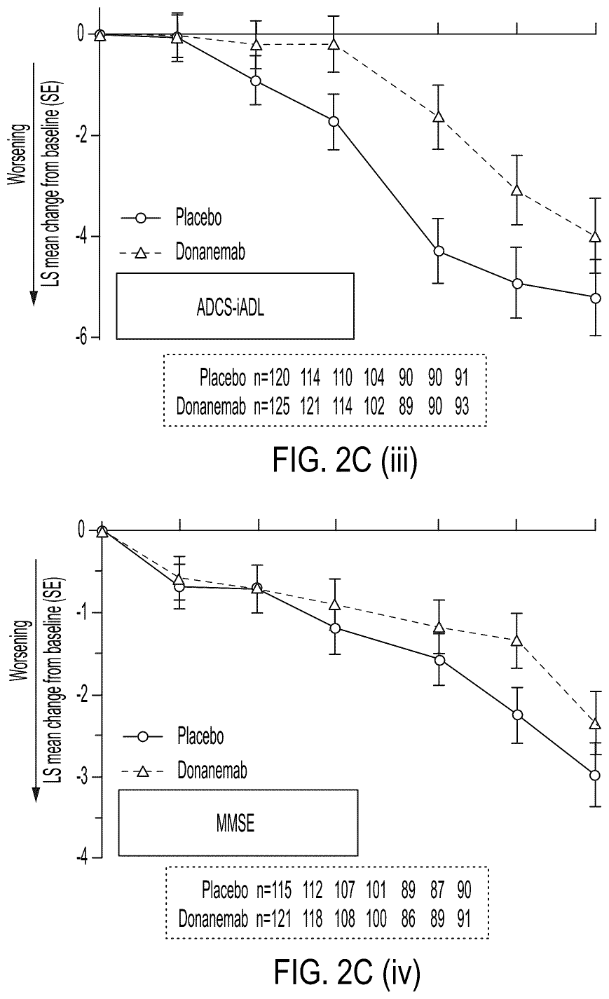

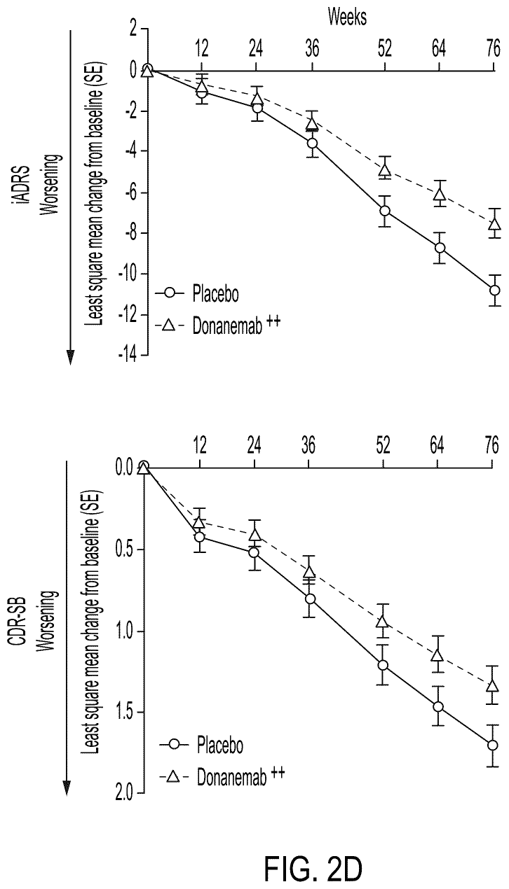

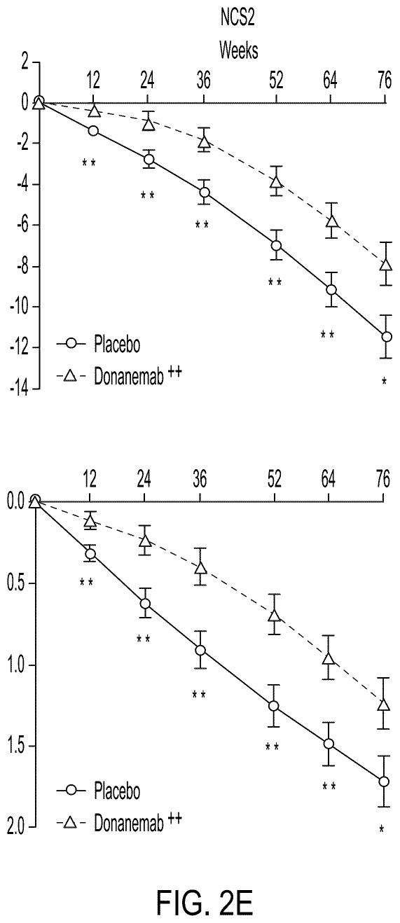

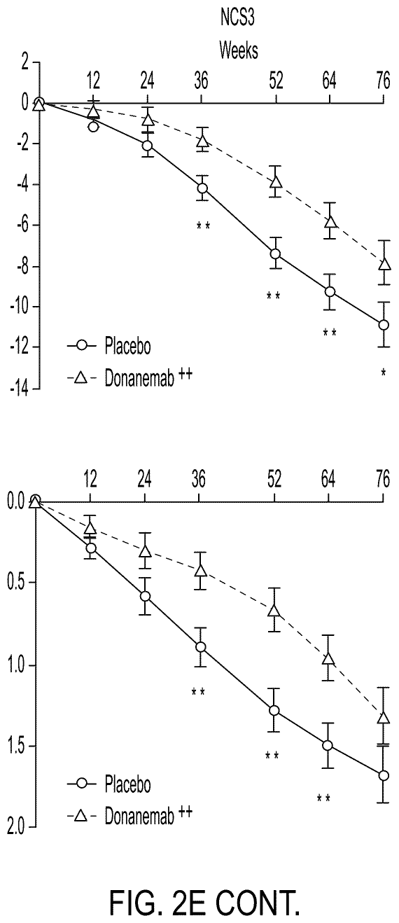

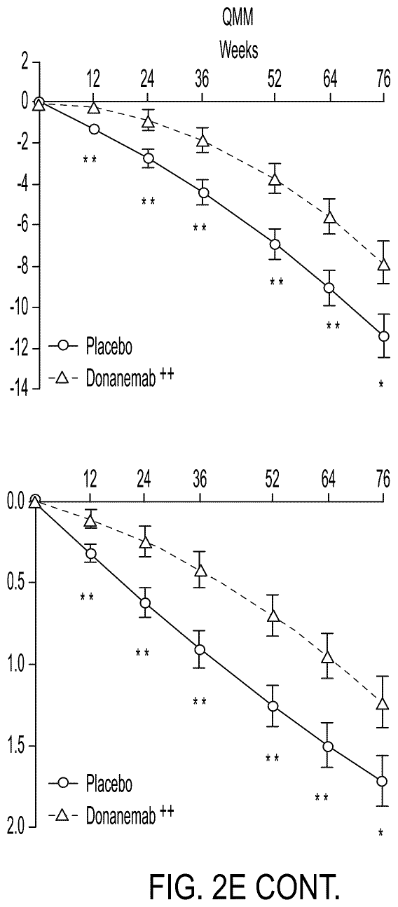

Primary Outcome: Donanemab showed significant slowing of decline in a composite measure of cognition and daily function in patients with early symptomatic Alzheimer's disease compared to placebo. Donanemab met the primary endpoint of change from baseline to 76 weeks in the Integrated Alzheimer's Disease Rating Scale (iADRS), slowing decline by 32% relative to placebo (FIGS. 2A-C), which was statistically significant. The iADRS is a clinical composite tool combining the cognitive measure ADAS-Cog₁₃ and functional measure ADCS-iADL, two commonly used measures in Alzheimer's disease. The change from baseline on iADRS at 76 weeks was −10.06 for placebo and −6.86 for donanemab treated patients (treatment difference: 3.20, 95% confidence interval [CI]: 0.12, 6.27; p=0.04) (FIGS. 2A-C and Table D). FIGS. 2A-C illustrate clinical outcomes for primary iADRS, and secondary CDR-SB, ADAS-Cog₁₃, ADCS-iADL, and MMSE. FIG. 2A shows the results for the primary outcome, the LS mean change from baseline to 76 weeks in iADRS score, analyzed with MMRM. FIG. 2B shows the percent slowing estimates from the MMRM model at the 18-month endpoint, and the Bayesian DPM model over the entire 18-month study. 95% credible intervals are shown. FIG. 2C shows the results for secondary outcomes, the LS mean change from baseline to 76 weeks in the (i) CDR-SB, (ii) ADAS-Cog₁₃, (iii) ADCS-iADL, and (iv) MMSE scores, analyzed with MMRM. In FIGS. 2A-C, A=Difference; W=Week; iADRS=integrated Alzheimer's Disease Rating Scale; ADAS-Cog₁₃=Alzheimer's Disease Assessment Scale-Cognitive subscale; ADCS-iADL=Alzheimer's Disease Cooperative Study-instrumental Activities of Daily Living scale; CDR-SB=Clinical Dementia Rating Scale Sum of Boxes; MMSE=Mini Mental State Examination; MMRM=Mixed Models for Repeated Measures; DPM=Disease Progression Model; LS=Least Squares; CI=Confidence Interval; n=Number of participants; SE=Standard Error.

FIG. 2D illustrates clinical results for primary iADRS and secondary CDR-SB outcomes using a Bayesian model of disease progression from TRAILBLZER-ALZ (AACG study, Example 2). In FIG. 2D, iADRS=Integrated Alzheimer's Disease rating Scale; CDR-SB=Clinical Dementia Rating−Sum of Boxes; ++ indicates posterior probability of at least 0% slowing >99%.

FIG. 2E shows the frequentist analyses of the clinical results of Example 2 for iADRS primary efficacy outcomes and CDR-SB secondary outcomes (*=p<0.05 vs. placebo; **=p<0.01 vs. placebo) using Natural Cubic Spline with 2 degrees of freedom (NCS2), Natural Cubic Spline with 3 degrees of freedom (NCS3), and quadratic mixed model (QMM) from TRAILBLZER-ALZ (AACG study, Example 2). The Natural Cubic Spline (NCS) model provides a type of smoothing function to the data and can adequately estimate longitudinal trajectories under a variety of shapes (e.g., linear, quadratic, etc.) for each treatment group. The degrees of freedom of the model can be prespecified to establish the level of smoothing of the data. The quadratic mixed model has many similar features to the MMRM but makes additional assumptions on the estimates of the longitudinal mean values such that the longitudinal trajectory of each treatment arm is smoothed over the scheduled or observed visit time to allow for a linear or quadratic shape. In FIG. 2E, iADRS=Integrated Alzheimer's Disease rating Scale; CDR-SB=Clinical Dementia Rating−Sum of Boxes; ++ indicates posterior probability of at least 0% slowing >99%, NCS2=Natural Cubic Spline with 2 degrees of freedom; NCS3=Natural Cubic Spline with 3 degrees of freedom; QMM=quadratic mixed model.

TABLE C
Shows the tabulated data for prespecified (MMRM and Bayesian DPM) and
exploratory (NCS2, NCS3, and QMM) statistical methods relative to placebo
for the results of the clinical study of Example 2 (study AACG).

| | Prespecified Methods | | Exploratory Methods | | |
|---|---|---|---|---|---|
| | MMRM | DPM ^{a} | NCS2 ^{a} | NCS3 | QMM |
| | LS Mean | LS Mean | LS Mean | LS Mean | LS Mean |
| | Change | Change | Change | Change | Change |
| | Difference | Difference | Difference | Difference | Difference |
| | (SE) | (SE) | (SE) | (SE) | (SE) |
| Measure | % Slowing | % Slowing | % Slowing | % Slowing | % Slowing |
| iADRS | 3.20 (1.560) * | 3.31 (0.875) ++ | 3.56 (1.464) * | 3.04 (1.522) * | 3.57 (1.461) * |
| (primary) | 31.8 | 30.4 | 31.2 | 28.0 | 31.4 |
| CDR-SB | −0.36 (0.239) | 0.37 (0.150) ++ | −0.48 (0.218) * | −0.36 (0.237) | −0.48 (0.218) * |
| | 22.6 | 21.6 | 27.8 | 21.6 | 28.2 |
| ADAS-Cog₁₃ | −1.86 (0.898) * | 1.67 (0.547) ++ | −1.71 (0.813) * | −1.92 (0.873) * | 1.71 (0.812) * |
| | 38.9 | 32.1 | 32.1 | 36.0 | 32.0 |
| ADCS-iADL | 1.21 (1.009) | 1.72 (0.554) ++ | 1.74 (0.956) | 1.10 (1.000) | 1.74 (0.955) |
| | 23.4 | 30.0 | 28.2 | 19.6 | 28.4 |
| MMSE | 0.64 (0.525) | 0.88 (0.461) ++ | 0.81 (0.461) | 0.67 (0.504) | 0.81 (0.462) |
| | 21.3 | 25.6 | 27.7 | 21.0 | 27.7 |

Abbreviations: AchEI = acetylcholinesterase inhibitor; ADAS-Cog₁₃ = Alzheimer's Disease Assessment Scale - 13-item Cognitive subscale; ADCS-iADL = Alzheimer's Disease Cooperative Study - instrumental Activities of Daily Living subscale; CDR-SB = Clinical Dementia Rating Scale - Sum of Boxes; CI = confidential interval; iADRS = integrated Alzheimer's Disease Rating Scale; LS = least squares; MMSE = Mini-Mental State Examination; NCS2 = natural cubic spline with 2 degrees of freedom; NCS3 = natural cubic spline with 3 degrees of freedom; Pr = probability; QMM = quadratic mixed model; SE = standard error; SUVr = standardized uptake value ratio.
For DPM,
++ indicates posterior probability of at least 0% slowing >99%; for all other analyses,
* p < .05;
** p < .01 for treatment effect of donanemab versus placebo.
^{a} Note that DPM and NCS2 analyses shown here included age, AChEI use at baseline, and pooled investigative site in the model to be consistent with MMRM analysis, while the DPM and NCS2 analyses included in the Study AACG study report only adjusted for age and AChEI use at baseline.

TABLE D
Primary iADRS, and Secondary CDR-SB, ADAS-Cog₁₃, ADCS-iADL, and MMSE Clinical Outcomes

| | | | | |
|---|---|---|---|---|
| iADRS | CDR-SB | ADAS-Cog₁₃ | ADCS-iADL | MMSE |

Week 12

| | | | | | | | | | | |
|---|---|---|---|---|---|---|---|---|---|---|
| Placebo, LS mean | −0.07 | (0.684) | 0.26 | (0.126) | −0.00 | (0.438) | −0.08 | (0.455) | −0.68 | (0.272) |
| change (SE) | | | | | | | | | | |
| Donanemab, LS | 0.42 | (0.673) | 0.21 | (0.124) | −0.40 | (0.433) | −0.02 | (0.448) | −0.58 | (0.267) |
| mean change (SE) | | | | | | | | | | |
| LS mean change | 0.49 | (−1.24, 2.22) | −0.05 | (−0.37, 0.27) | −0.40 | (−1.51, 0.71) | 0.06 | (−1.07, 1.20) | 0.10 | (−0.58, 0.78) |
| difference (95% CI) | | | | | | | | | | |

Week 24

| | | | | | | | | | | |
|---|---|---|---|---|---|---|---|---|---|---|
| Placebo, LS mean | −1.18 | (0.666) | 0.39 | (0.123) | 0.18 | (0.451) | −0.91 | (0.481) | −0.70 | (0.295) |
| change (SE) | | | | | | | | | | |
| Donanemab, LS | 0.12 | (0.663) | 0.26 | (0.123) | −0.33 | (0.450) | −0.21 | (0.478) | −0.71 | (0.292) |
| mean change (SE) | | | | | | | | | | |
| LS mean change | 1.30 | (−0.38, 2.99) | −0.13 | (−0.44, 0.18) | −0.51 | (−1.66, 0.65) | 0.69 | (−0.52, 1.91) | −0.01 | (−0.77, 0.74) |
| difference (95% CI) | | | | | | | | | | |

Week 36

| | | | | | | | | | | |
|---|---|---|---|---|---|---|---|---|---|---|
| Placebo, LS mean | −3.17 | (0.730) | 0.83 | (0.139) | 1.36 | (0.445) | −1.73 | (0.556) | −1.19 | (0.314) |
| change (SE) | | | | | | | | | | |
| Donanemab, LS | −0.73 | (0.732) | 0.26 | (0.140) | 0.53 | (0.446) | −0.19 | (0.558) | −0.89 | (0.312) |
| mean change (SE) | | | | | | | | | | |
| LS mean change | 2.44 | (0.55, 4.33) | −0.56 | (−0.92, −0.20) | −0.84 | (−1.98, 0.30) | 1.54 | (0.09, 2.99) | 0.30 | (−0.51, 1.11) |
| difference (95% CI) | | | | | | | | | | |

Week 52

| | | | | | | | | | | |
|---|---|---|---|---|---|---|---|---|---|---|
| Placebo, LS mean | −6.70 | (0.929) | 1.21 | (0.160) | 2.37 | (0.536) | −4.28 | (0.635) | −1.56 | (0.321) |
| change (SE) | | | | | | | | | | |
| Donanemab, LS | −3.03 | (0.933) | 0.62 | (0.160) | 1.53 | (0.540) | −1.64 | (0.637) | −1.17 | (0.321) |
| mean change (SE) | | | | | | | | | | |
| LS mean change | 3.67 | (1.19, 6.15) | −0.59 | (−1.01, −0.16) | −0.84 | (−2.25, 0.58) | 2.64 | (0.96, 4.33) | 0.40 | (−0.44, 1.23) |
| difference (95% CI) | | | | | | | | | | |

Week 64

| | | | | | | | | | | |
|---|---|---|---|---|---|---|---|---|---|---|
| Placebo, LS mean | −8.34 | (1.038) | 1.33 | (0.171) | 3.30 | (0.621) | −4.91 | (0.689) | −2.25 | (0.339) |
| change (SE) | | | | | | | | | | |
| Donanemab, LS | −4.92 | (1.038) | 1.06 | (0.170) | 1.87 | (0.619) | −3.07 | (0.687) | −1.34 | (0.335) |
| mean change (SE) | | | | | | | | | | |
| LS mean change | 3.42 | (0.63, 6.21) | −0.27 | (−0.72, 0.18) | −1.43 | (−3.09, 0.23) | 1.85 | (0.01, 3.69) | 0.91 | (0.02, 1.79) |
| difference (95% CI) | | | | | | | | | | |

Week 76

| | | | | | | | | | | |
|---|---|---|---|---|---|---|---|---|---|---|
| Placebo, LS mean | −10.06 | (1.141) | 1.58 | (0.178) | 4.77 | (0.660) | −5.20 | (0.743) | −2.98 | (0.390) |
| change (SE) | | | | | | | | | | |
| Donanemab, LS | −6.86 | (1.135) | 1.22 | (0.176) | 2.91 | (0.659) | −3.98 | (0.738) | −2.35 | (0.386) |
| mean change (SE) | | | | | | | | | | |
| LS mean change | 3.20 | (0.12, 6.27) | −0.36 | (−0.83, 0.12) | −1.86 | (−3.63, −0.09) | 1.21 | (−0.77, 3.20) | 0.64 | (−0.40, 1.67) |
| difference (95% CI) | | | | | | | | | | |

Results for the mean change from baseline for the primary iADRS, and secondary ADAS-Cog₁₃, ADCS-iADL, CDR-SB, and MMSE clinical outcomes, analyzed with MMRM. iADRS = integrated Alzheimer's Disease Rating Scale; ADAS-Cog₁₃ = Alzheimer's Disease Assessment Scale-Cognitive subscale; ADCS-iADL = Alzheimer's Disease Cooperative Study-instrumental Activities of Daily Living scale; CDR-SB = Clinical Dementia Rating Scale Sum of Boxes; MMSE = Mini Mental State Examination; MMRM = Mixed Models for Repeated Measures; LS = Least Squares; CI = Confidence Interval; SE = Standard Error

Percent slowing estimates of disease progression relative to placebo, from the MMRM model at the 18-month endpoint and the Bayesian DPM over the entire 18-months, showed slowing of decline in the iADRS with both methods (FIG. 2B). The posterior probability of at least 25% slowing of disease progression relative to placebo on the iADRS was calculated as 0.78 from the Bayesian DPM.

Secondary Outcome: Donanemab also showed consistent improvements in all prespecified secondary endpoints measuring cognition and function compared to placebo but did not reach nominal statistical significance on every secondary endpoint. In the donanemab group, the observed difference from placebo in change from baseline at 76 weeks for CDR-SB was −0.36 (95% CI: −0.83 to 0.12), for ADAS-Cog₁₃ was −1.86 (95% CI: −3.63 to −0.09), for ADCS-iADL was 1.21 (95% CI: −0.77 to 3.20), and for MMSE was 0.64 (95% CI: −0.40 to 1.67) (FIG. 2C and Table E).

TABLE E
Primary iADRS, and secondary CDR-SB, ADAS-Cog₁₃, ADCS-iADL, and MMSE clinical outcomes

| | | | | |
|---|---|---|---|---|
| iADRS | CDR-SB | ADAS-Cog₁₃ | ADCS-iADL | MMSE |

Week 12

| | | | | | | | | | | |
|---|---|---|---|---|---|---|---|---|---|---|
| Placebo, LS mean | −0.07 | (0.684) | 0.26 | (0.126) | −0.00 | (0.438) | −0.08 | (0.455) | −0.68 | (0.272) |
| change (SE) | | | | | | | | | | |
| Donanemab, LS | 0.42 | (0.673) | 0.21 | (0.124) | −0.40 | (0.433) | −0.02 | (0.448) | −0.58 | (0.267) |
| mean change (SE) | | | | | | | | | | |
| LS mean change | 0.49 | (−1.24, 2.22) | −0.05 | (−0.37, 0.27) | −0.40 | (−1.51, 0.71) | 0.06 | (−1.07, 1.20) | 0.10 | (−0.58, 0.78) |
| difference (95% CI) | | | | | | | | | | |

Week 24

| | | | | | | | | | | |
|---|---|---|---|---|---|---|---|---|---|---|
| Placebo, LS mean | −1.18 | (0.666) | 0.39 | (0.123) | 0.18 | (0.451) | −0.91 | (0.481) | −0.70 | (0.295) |
| change (SE) | | | | | | | | | | |
| Donanemab, LS | 0.12 | (0.663) | 0.26 | (0.123) | −0.33 | (0.450) | −0.21 | (0.478) | −0.71 | (0.292) |
| mean change (SE) | | | | | | | | | | |
| LS mean change | 1.30 | (−0.38, 2.99) | −0.13 | (−0.44, 0.18) | −0.51 | (−1.66, 0.65) | 0.69 | (−0.52, 1.91) | −0.01 | (−0.77, 0.74) |
| difference (95% CI) | | | | | | | | | | |

Week 36

| | | | | | | | | | | |
|---|---|---|---|---|---|---|---|---|---|---|
| Placebo, LS mean | −3.17 | (0.730) | 0.83 | (0.139) | 1.36 | (0.445) | −1.73 | (0.556) | −1.19 | (0.314) |
| change (SE) | | | | | | | | | | |
| Donanemab, LS | −0.73 | (0.732) | 0.26 | (0.140) | 0.53 | (0.446) | −0.19 | (0.558) | −0.89 | (0.312) |
| mean change (SE) | | | | | | | | | | |
| LS mean change | 2.44 | (0.55, 4.33) | −0.56 | (−0.92, −0.20) | −0.84 | (−1.98, 0.30) | 1.54 | (0.09, 2.99) | 0.30 | (−0.51, 1.11) |
| difference (95% CI) | | | | | | | | | | |

Week 52

| | | | | | | | | | | |
|---|---|---|---|---|---|---|---|---|---|---|
| Placebo, LS mean | −6.70 | (0.929) | 1.21 | (0.160) | 2.37 | (0.536) | −4.28 | (0.635) | −1.56 | (0.321) |
| change (SE) | | | | | | | | | | |
| Donanemab, LS | −3.03 | (0.933) | 0.62 | (0.160) | 1.53 | (0.540) | −1.64 | (0.637) | −1.17 | (0.321) |
| mean change (SE) | | | | | | | | | | |
| LS mean change | 3.67 | (1.19, 6.15) | −0.59 | (−1.01, −0.16) | −0.84 | (−2.25, 0.58) | 2.64 | (0.96, 4.33) | 0.40 | (−0.44, 1.23) |
| difference (95% CI) | | | | | | | | | | |

Week 64

| | | | | | | | | | | |
|---|---|---|---|---|---|---|---|---|---|---|
| Placebo, LS mean | −8.34 | (1.038) | 1.33 | (0.171) | 3.30 | (0.621) | −4.91 | (0.689) | −2.25 | (0.339) |
| change (SE) | | | | | | | | | | |
| Donanemab, LS | −4.92 | (1.038) | 1.06 | (0.170) | 1.87 | (0.619) | −3.07 | (0.687) | −1.34 | (0.335) |
| mean change (SE) | | | | | | | | | | |
| LS mean change | 3.42 | (0.63, 6.21) | −0.27 | (−0.72, 0.18) | −1.43 | (−3.09, 0.23) | 1.85 | (0.01, 3.69) | 0.91 | (0.02, 1.79) |
| difference (95% CI) | | | | | | | | | | |

Week 76

| | | | | | | | | | | |
|---|---|---|---|---|---|---|---|---|---|---|
| Placebo, LS mean | −10.06 | (1.141) | 1.58 | (0.178) | 4.77 | (0.660) | −5.20 | (0.743) | −2.98 | (0.390) |
| change (SE) | | | | | | | | | | |
| Donanemab, LS | −6.86 | (1.135) | 1.22 | (0.176) | 2.91 | (0.659) | −3.98 | (0.738) | −2.35 | (0.386) |
| mean change (SE) | | | | | | | | | | |
| LS mean change | 3.20 | (0.12, 6.27) | −0.36 | (−0.83, 0.12) | −1.86 | (−3.63, −0.09) | 1.21 | (−0.77, 3.20) | 0.64 | (−0.40, 1.67) |
| difference (95% CI) | | | | | | | | | | |

Results for the mean change from baseline for the primary iADRS, and secondary ADAS-Cog₁₃, ADCS-iADL, CDR-SB, and MMSE clinical outcomes, analyzed with MMRM. iADRS = integrated Alzheimer's Disease Rating Scale; ADAS-Cog₁₃ = Alzheimer's Disease Assessment Scale-Cognitive subscale; ADCS-iADL = Alzheimer's Disease Cooperative Study-instrumental Activities of Daily Living scale; CDR-SB = Clinical Dementia Rating Scale Sum of Boxes; MMSE = Mini Mental State Examination; MMRM = Mixed Models for Repeated Measures; LS = Least Squares; CI = Confidence Interval; SE = Standard Error

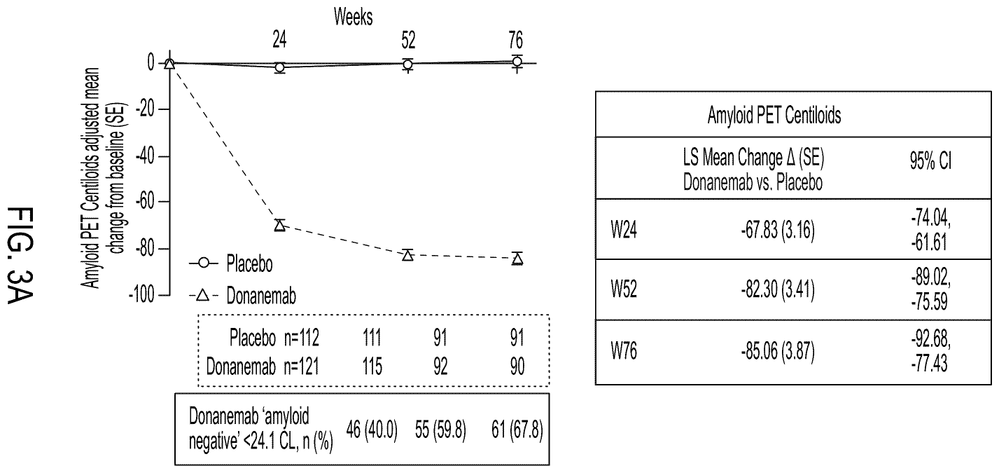

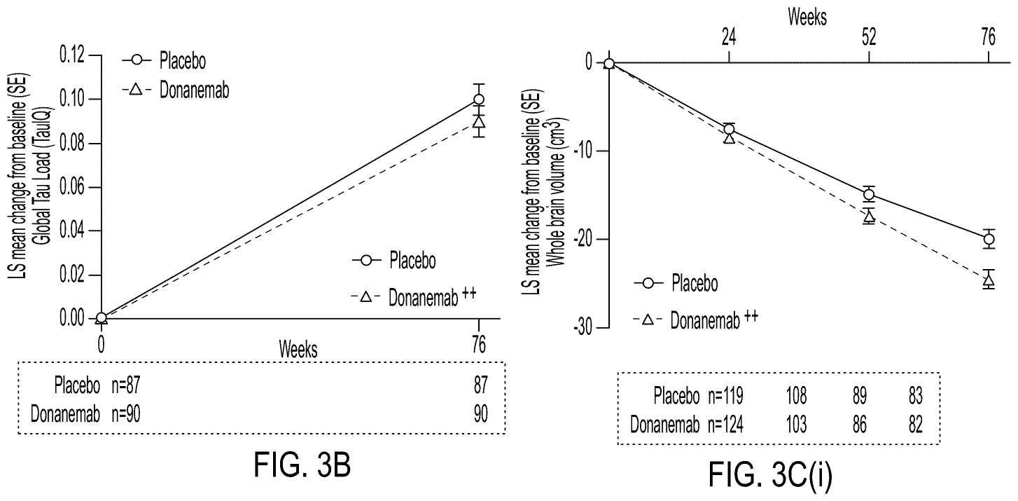

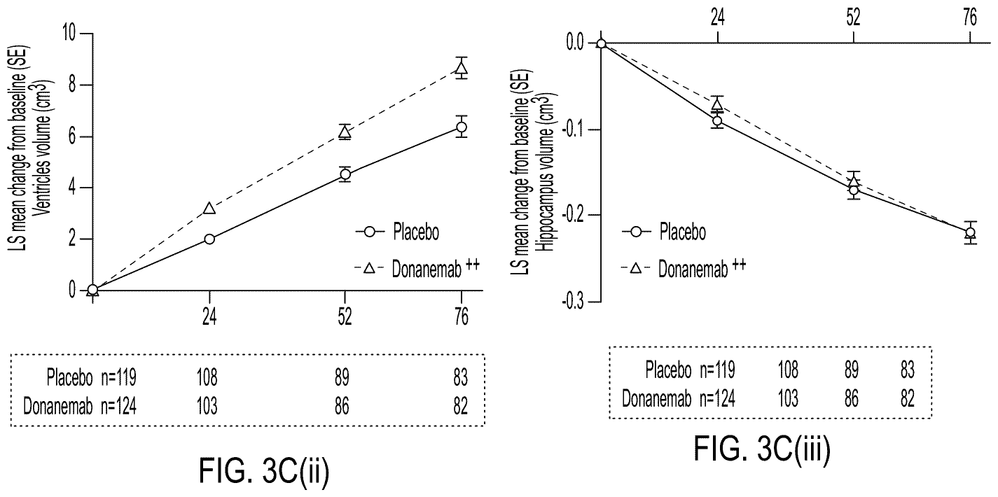

Biomarkers: By targeting N3pGlu Aβ, donanemab treatment has been shown to rapidly result in high levels of amyloid plaque removal, as measured by amyloid imaging. For PET amyloid, participants treated with donanemab showed an 85 CL amyloid plaque reduction at 76 weeks, compared with placebo (placebo=0.93, donanemab=−84.13) (FIG. 3A). A separation of 68 CL reduction was evident in the donanemab group by 24 weeks, compared with placebo (placebo=−1.82, donanemab=−69.64; 65% reduction from baseline in donanemab group). The percentage of participants that were ‘amyloid negative’ (defined as <24.1 CL amyloid plaque) in the donanemab group, at 24, 52, and 76 weeks was 40.0%, 59.8%, and 67.8%, respectively (FIG. 3A). Approximately 27% and 55% of donanemab participants dosed at week 28 and week 56, respectively, achieved adequate amyloid lowering to reduce to placebo infusion. In this study, patients stopped receiving donanemab and switched to placebo once their amyloid plaque level was below 25 centiloids for two consecutive measures or below 11 centiloids at any one measure.

Evaluation of global tau load assessed by F18-flortaucipir PET revealed no difference between groups from baseline to 76 weeks (FIG. 3B). Hippocampal volume change, assessed with vMRI, showed no difference between groups (FIG. 3C(iii)). There was a greater whole brain volume decrease and greater ventricular volume increase in participants treated with donanemab at 52, compared with placebo (FIG. 3C(i) & (ii)). FIGS. 3A-C show the outcomes for secondary biomarkers. FIG. 3A shows results for the secondary outcomes, change from baseline to 76 weeks, in brain amyloid plaque deposition, as measured by F18-florbetapir PET scan in centiloids (CL). FIG. 3B shows the global tau load, as measured by F18-flortaucipir PET scan. ‘Amyloid negative’/<24.1 CL=the average CL level for similar aged otherwise healthy individuals. FIG. 3C shows vMRI for (i) whole brain, (ii) ventricles, and (iii) hippocampus. In FIG. 3, A=Difference; W=Week; LS=Least squares; CI=Confidence Interval; CL=Centiloids; n=Number of participants; SE=Standard Error.

Adverse Events: There was no difference in the incidence of death or serious adverse events (SAEs) between donanemab and placebo groups. A total of 113 of 125 participants (90.4%) in the placebo group and 119 of 131 (90.8%) in the donanemab group had at least one treatment-emergent adverse event (TEAE) during the double-blind period in the safety population. The incidence of ARIA-E was significantly higher in the donanemab group (27%), compared with placebo (0.8%). Symptomatic ARIA-E was reported by 6.1% of all participants in the donanemab group (22% of participants with ARIA-E), compared with 0.8% in the placebo group. Most ARIA-E cases occurred by week 12 of dosing initiation. Serious symptomatic ARIA-E requiring hospitalization occurred in 2 participants treated with donanemab (1.5%). Both participants had symptoms of confusion and one reported difficulty expressing themselves, all of which fully resolved. ARIA-E fully resolved in both cases, with a mean ARIA-E resolution time of 18 weeks. The incidence of superficial siderosis (a type of ARIA with hemorrhage (ARIA-H)) of the central nervous system, nausea, and infusion-related reactions (IRR) were all significantly higher in the donanemab group, compared with placebo. Treatment discontinuation due to ARIA-E occurred with 7 participants (5.3%) in the donanemab group; 2 (1.5%) discontinued the study due to ARIA-E. No brain macrohemorrhages were seen in either group. IRR were reported in 7.6% of participants on donanemab and 0% on placebo. Serious IRR or hypersensitivity occurred in 3 participants (2.3%) treated with donanemab. The incidence of treatment-emergent anti-drug antibodies (TE-ADAs) in participants treated with donanemab was approximately 90%.

These results show that in an amyloid-plaque-specific intervention in patients with early symptomatic AD, amyloid removal in the donanemab arm was accompanied by a slowing of disease progression, compared with placebo. The 3.20 treatment difference at 76 weeks on the iADRS scale should be interpreted in the context of not only the score range across the entire disease spectrum (0 to 144) but also, importantly, the dynamic range of iADRS within the participant population (26 points) and the decline in the placebo group (−10.06).

The results provided here are unexpected and surprising in several aspects. The dosing regimen of donanemab provides high amounts of amyloid removal early in the trial with almost 60% of participants having an ‘amyloid negative’ scan by 52 weeks. This is the first study to screen all participants with F18-flortaucipir PET scans, likely narrowing the range of underlying pathology, which in turn, likely decreased variance of the clinical decline.

The tau PET screening of the patients excluded subjects with high tau. Patients with high tau may be less responsive to anti-amyloid treatment or may have a disease that is more resistant to anti-amyloid treatments.

As proposed by the European Prevention of Alzheimer's Dementia project, analysis of treatment differences for the iADRS, ADAS-Cog₁₃, ADCS-iADL, CDR-SB, and MMSE scores, was performed using a relatively novel disease progression model. Given better sensitivity for detecting treatment effects (Solomon et al., “European Prevention of Alzheimer's Dementia Longitudinal Cohort Study (EPAD LCS): Study Protocol,” BMJ Open 8:e021017 (2018), which is hereby incorporated by reference in its entirety), this model can allow for substantial gains in statistical power (Wang et al., “A Novel Cognitive Disease Progression Model for Clinical Trials in Autosomal-dominant Alzheimer's disease,” Statistics in Medicine 37:3047-55 (2018), which is hereby incorporated by reference in its entirety) and in this trial revealed similar estimates of disease slowing to the single point-estimate of the MMRM model.

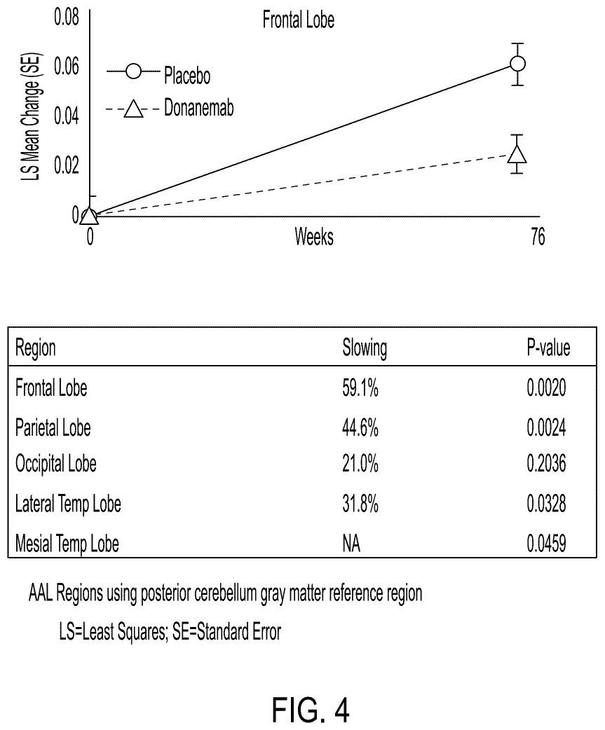

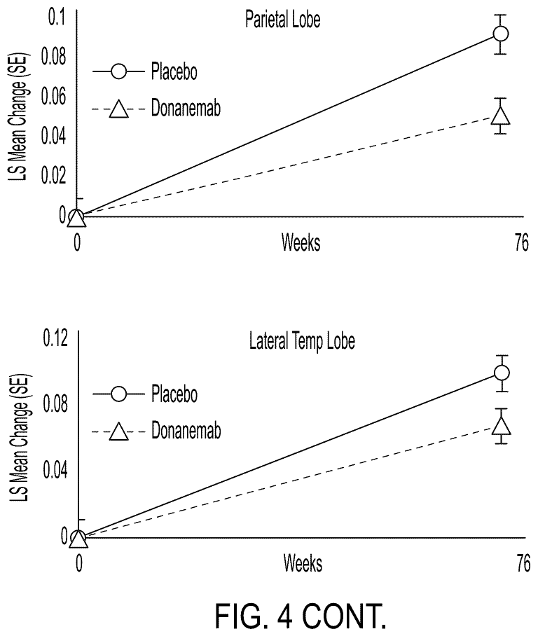

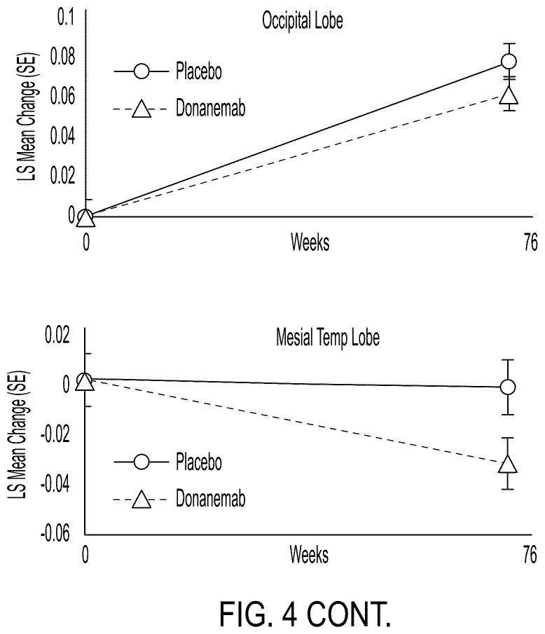

Regarding the observed lack of treatment effect on global tau load, it is conceivable that tau changes by PET will lag substantially relative to amyloid changes and that an 18-month time is too short to detect imaging changes. Modelling in autosomal dominant subjects suggest a lag of 10-20 years from first detectable PET amyloid changes and first detectable tau PET changes (Barthelemy et al., “A Soluble Phosphorylated Tau Signature Links Tau, Amyloid and the Evolution of Stages of Dominantly Inherited Alzheimer's Disease,” Nat. Med. 26:398-407 (2020), which is hereby incorporated by reference in its entirety). The lack of impact on global tau may prompt questions about whether targeting amyloid-β reduction affects biological disease progression. However, additional pre-specified analyses of brain regions suggest a reduction in tau accumulation in various regions of the brain (e.g., frontal, parietal, occipital, and temporal lobe regions), in the donanemab group compared with placebo (FIG. 4).

A robust decrease or prevention of further increase of tau accumulation is seen in, e.g., the frontal lobes of the brain. Occipital lobe has some of the highest baseline signal and may therefore provide a ceiling effect on the ability to show a decrease in the increasing tau load. FIG. 4 shows regional SUVr analyses of tau accumulation with cerebellar gray reference. The frontal lobe tau load, as measured by F18-flortaucipir, using a cerebellar reference region is correlated with the iADRS and CDR-SB change over the following 76 weeks in symptomatic early AD subjects. FIG. 5 shows that lower frontal tau burden is associated with less decline in the patients. High frontal lobe tau burden is associated with a fast decline in the patients. In other words, patients with low frontal lobe tau burden experience slower decline (as measured by iADRS or CDR-SB) as compared to patients with high frontal lobe tau burden.

This measurement reflects global changes in tau load and further exploration may show subregions that could be more sensitive to change. Optimal methods for region selection and analysis for quantifying tau changes and response to therapy remain in their infancy.

There was no significant change in hippocampal volume in contrast with the recent BACE inhibitor studies that showed significant volume changes (Wessels et al., “Efficacy and Safety of Lanabecestat for Treatment of Early and Mild Alzheimer Disease: The AMARANTH and DAYBREAK-ALZ Randomized Clinical Trials,” JAMA Neurology 77:199-209 (2020), which is hereby incorporated by reference in its entirety). Observations of greater whole brain volume decrease, and greater ventricular volume increase with donanemab treatment, compared with placebo, may be interpreted in the context of protein removal rather than atrophy. Global volumetric MRI changes are typically attributed to atrophy in natural history studies of AD, but it remains unclear if they represent true atrophy in the context of rapid structural removal of protein aggregates, as seen in this study, and in another anti-amyloid therapy study (Sur et al., “BACE Inhibition Causes Rapid, Regional, and Non-progressive Volume Reduction in Alzheimer's Disease Brain,” Brain 143:3816-26 (2020), which is hereby incorporated by reference in its entirety).

ARIA-E and ARIA-H have been associated with amyloid plaque-removing treatments (Sperling et al., “Amyloid-related imaging abnormalities in amyloid-modifying therapeutic trials: Recommendations from the Alzheimer's Association Research Roundtable Workgroup,” Alzheimer's & Dementia 7:367-85 (2011); Sevigny et al., “The Antibody Aducanumab Reduces Aβ Plaques in Alzheimer's Disease,” Nature 537:50-6 (2016); Ostrowitzki et al., “Mechanism of Amyloid Removal in Patients With Alzheimer Disease Treated With Gantenerumab,” Archives of Neurology 69:198-207 (2012); Salloway et al., “Two Phase 3 Trials of Bapineuzumab in Mild-to-Moderate Alzheimer's Disease,” New England Journal of Medicine 370:322-33 (2014); Salloway et al., “A Phase 2 Multiple Ascending Dose Trial of Bapineuzumab in Mild to Moderate Alzheimer Disease,” Neurology 73:2061-70 (2009); and Sperling et al., “Amyloid-related Imaging Abnormalities in Patients with Alzheimer's Disease Treated with Bapineuzumab: A Retrospective Analysis,” Lancet Neurol. 11:241-9 (2012), which are hereby incorporated by reference in their entireties).

In the Phase 1b study, the incidence of ARIA-E was 26.1% among participants treated with donanemab, with 2 participants reporting symptomatic ARIA-E (4.3%). In this study, a similar incidence of ARIA-E was found in the donanemab group (27%), with 6.1% reporting symptomatic ARIA-E. Incidence of ARIA-E was more prevalent in APOE4 carriers as seen in other trials of plaque targeting antibodies (Sevigny et al., “The Antibody Aducanumab Reduces Aβ Plaques in Alzheimer's Disease,” Nature 2016; 537:50-6; Ostrowitzki et al., “Mechanism of Amyloid Removal in Patients With Alzheimer Disease Treated With Gantenerumab,” Archives of Neurology 69:198-207; Salloway et al., “Two Phase 3 Trials of Bapineuzumab in Mild-to-Moderate Alzheimer's Disease,” NEJM 2014; 370:322-33 (2014); and Sperling et al., “Amyloid-related Imaging Abnormalities in Patients with Alzheimer's Disease Treated with Bapineuzumab: A Retrospective Analysis,” Lancet Neurol. 11:241-9 (2012), which is hereby incorporated by reference in its entirety). The incidence of treatment-emergent anti-drug antibodies (TE-ADAs) in participants treated with donanemab (approximately 90%) was similar to findings from Phase 1 (>85%).

These results demonstrate that in participants with early symptomatic AD, treatment with donanemab resulted in amyloid plaque clearance, and a slowing of cognitive and functional decline as measured by the iADRS scale.

### Example 4: Efficacy Associated with Baseline Tau PET Patient Stratification

The anti-N3pGlu Aβ antibody, donanemab, is found to be most efficacious in a subject with the lowest baseline flortaucipir level. The antibody may be less efficacious in subjects having high tau (>1.46 SUVr). In other words, subjects having high tau (>1.46 SUVr) may be less responsive to Aβ therapies, particularly, therapies based on anti-N3pGlu antibodies, including, e.g., donanemab.

Tau levels (e.g., for the purpose of stratification of the human subject suffering from AD) are determined based on an initial visual assessment of a flortaucipir scan, followed by a quantitative analysis. Visual assessment relies on a 3-tier read (tAD−, tAD+, tAD++) based on the presence of tracer uptake in specific regions of the neocortex. Quantitative analysis refers to calculation of SUVr, which represents counts within a specific target region of interest in the brain (e.g., multiblock barycentric discriminant analysis or MUBADA) when compared with a reference region (parametric estimate of reference signal intensity or PERSI). Lower SUVr values indicate less tau burden while higher SUVr values indicate greater tau burden.

A scan in the low to moderate tau group (e.g., having a SUVr from ≥1.10 to ≤1.46), as shown in Table F, is eligible for administration of anti-N3pGlu Aβ antibodies in study AACG.

VisualAssessment: Methods for visual assessment of human subjects are described in Fleisher et al., “Positron Emission Tomography Imaging With [¹⁸F]flortaucipir and Postmortem Assessment of Alzheimer Disease Neuropathologic Changes,” JAMA Neurol. 77(7):829-839 (2020), which is hereby incorporated by reference in its entirety. Briefly, a flortaucipir scan is negative (tAD−) if there is no increased neocortical tracer activity in any region of the brain or activity is isolated to the frontal lobe or regions of the temporal lobe that do not include the posterolateral temporal (PLT) region. Positive scans fall into two categories based on the regions of increased neocortical tracer activity. A flortaucipir scan that has neocortical tracer activity limited to the posterolateral temporal (PLT) or occipital regions is classified as tAD+.

Finally, if the flortaucipir scan shows increased tracer activity in the parietal or precuneus region or there is activity in the frontal region along with activity in PLT or occipital regions, it is classified as tAD++. Quantitative analysis is performed on all tAD+ and tAD++ scans.

Quantitative Analysis: Quantitative analysis is accomplished through an automated image processing pipeline. A previously developed neocortical target volumes of interest (VOI) (MUBADA, see Devous et al., “Test-Retest Reproducibility for the Tau PET Imaging Agent Flortaucipir F18,” J. Nucl. Med 2018; 59:937-943 (2018), which is hereby incorporated by reference in its entirety) is applied to each scan and the derived counts are normalized to a patient-specific reference region (PERSI). Other target and reference regions are also extracted through the pipeline. The PERSI reference region is a subject-specific, data-driven technique that identifies voxels with nonspecific flortaucipir uptake within an atlas-defined white matter region (see, e.g., Southekal et al, “Flortaucipir F18 Quantitation Using Parametric Estimation of Reference Signal Intensity,” J. Nucl. Med 59:944-951 (2018), which is hereby incorporated by reference in its entirety)). The MUBADA target region was developed using a statistical method to maximize separation of diagnostic groups based on image characteristics (see Devous et al, “Test-Retest Reproducibility for the Tau PET Imaging Agent Flortaucipir F18,” J. Nucl. Med 59:937-943 (2018), which is hereby incorporated by reference in its entirety). When applied to F18-flortaucipir images from a large dataset of 202 subjects (55 Aβ− older cognitively normal, 43 Aβ− MCI, 54 Aβ+ MCI, 16 Aβ− AD, and 34 Aβ+ AD) the analysis yielded 2 dimensions (aka components). The first dimension (which explained 95% of the variance) provided maximal separation of groups by diagnosis and amyloid status and was converted into a VOI that is now referred to as the MUBADA VOI (see, e.g., Devous et al., “Test-Retest Reproducibility for the Tau PET Imaging Agent Flortaucipir F18,” J. Nucl. Med 2018; 59:937-943 (2018), which is hereby incorporated by reference in its entirety)).

The MUBADA VOI ratioed to the PERSI reference region was then applied to 204 subjects and the resulting values were divided into 4 tau-burden quartiles: 1) very low; 2) low; 3) moderate; and 4) high. The cutoff SUVr values separating very low and low was 1.10; low and moderate was 1.23; moderate and high was 1.46. These values were used to screen subjects according to the algorithm described above.

Subjects with tAD+ and tAD++ scans with SUVr>1.46 were not administered anti-N3pGlu Aβ antibodies based on the hypothesis that cognitive decline in patients with high tau was driven primarily by their tauopathy and thus would not respond to anti-amyloid therapy.

TABLE F
Tau Assessment Criteria

| | Quantitative |
|---|---|
| | Classification |
| Visual Classification Criteria | Criteria (PERSI) |
| tAD− | Not measured |
| no increased neocortical activity | |
| or activity isolated to the MLT, | |
| ALT or frontal regions | |
| tAD+ | Very low tau |
| increased neocortical activity | SUVr < 1.10 |
| in PLT or occipital | Low to moderate tau |
| | 1.10 ≤ SUVr ≤ 1.46 |
| | High tau |
| | SUVr > 1.46 |
| tAD++ | Very low tau |
| increased neocortical activity | SUVr < 1.10 |
| in parietal (precuneus), or | Low to moderate tau |
| in frontal region in combination with | 1.10 ≤ SUVr ≤ 1.46 |
| PLT/occipital/parietal | High tau |
| | SUVr > 1.46 |

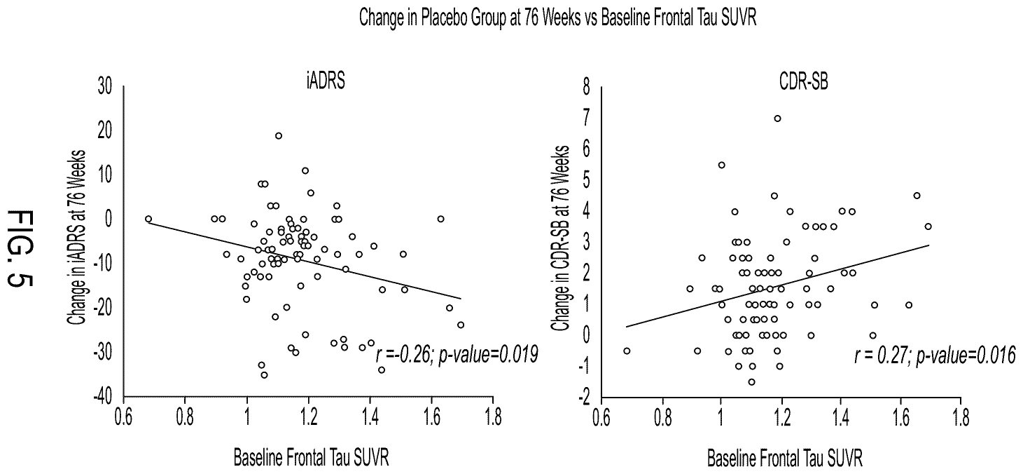

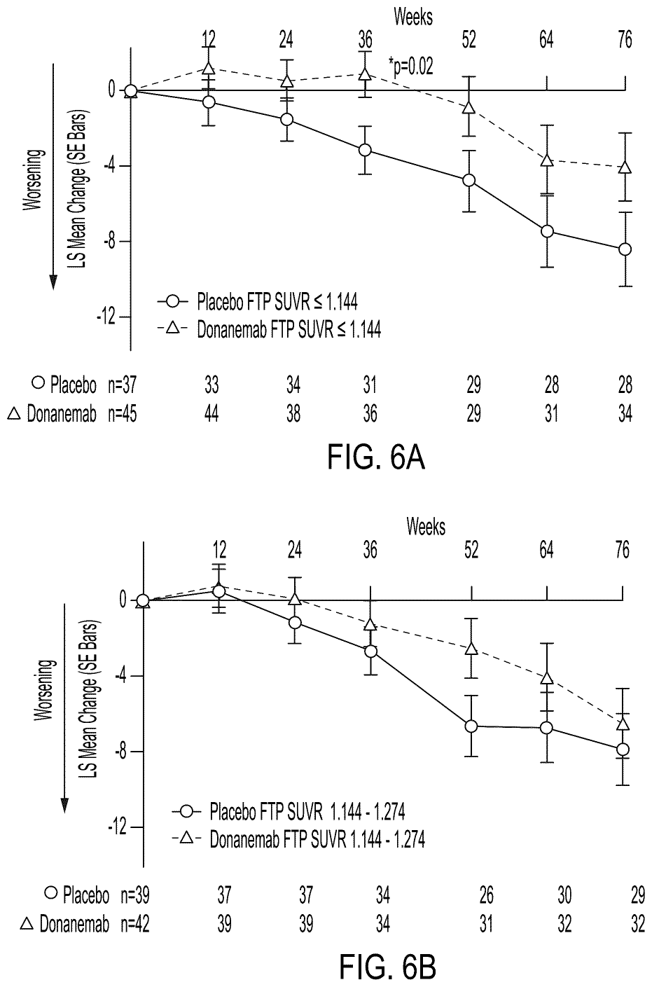

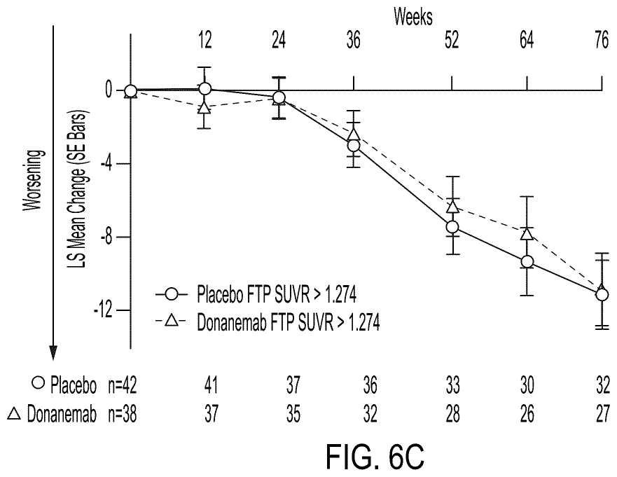

As shown in FIGS. 6A-C below, the anti-N3pGlu Aβ antibody, donanemab, was found to be most efficacious in the treatment sub-group with the lowest baseline flortaucipir signal. Based on FIG. 6, it may be hypothesized that patients having high tau (>1.46 SUVr) are unlikely to be responsive to therapy.

The data demonstrate that the anti-N3pGlu Aβ antibody, donanemab, was most efficacious in human subjects having a tau level less than or equal to about 1.14 SUVr or less than or equal to about 1.27 SUVr (FIGS. 6A and 6B). The change in scale scores was not statistically significant in the donanemab treatment group compared to placebo in the graphs on the farthest right, defined by baseline tau PET SUVr values greater than 1.274 SUVr (FIG. 6C). FIGS. 6A-C show baseline tau subgroup analysis based on iADRS (FTP=F18-flortaucipir).

### Example 5: Efficacy and Safety Associated with Carriers of the Allele Apolipoprotein E4 (APOE4)

The phase 2 clinical trial (NCT03367403; clinicaltrials.gov)—disclosed above in Examples 2, 3, and 4—also included examination of efficacy and safety of the anti-N3pGlu Aβ antibody (donanemab) in the subgroup of participants that have one or two alleles of APOE4.

This phase 2 clinical trial was a randomized, placebo-controlled, double-blind, multi-center Phase 2 study assessing the safety, tolerability, and efficacy of donanemab in patients with early symptomatic AD. The clinical change from baseline to 76 weeks was assessed for all enrolled patients with intermediate tau pathology levels using the Integrated AD Rating Scale (iADRS; primary endpoint), a composite tool measuring cognition and daily function, and the Clinical Dementia Rating Scale-Sum of Boxes (CDR-SB; secondary endpoint). Baseline characteristics showed that 72.5% and 74.2% of patients treated respectively with donanemab or placebo were APOE4 carriers. Additional analysis of iADRS and key secondary endpoints were conducted focusing on this subgroup population.

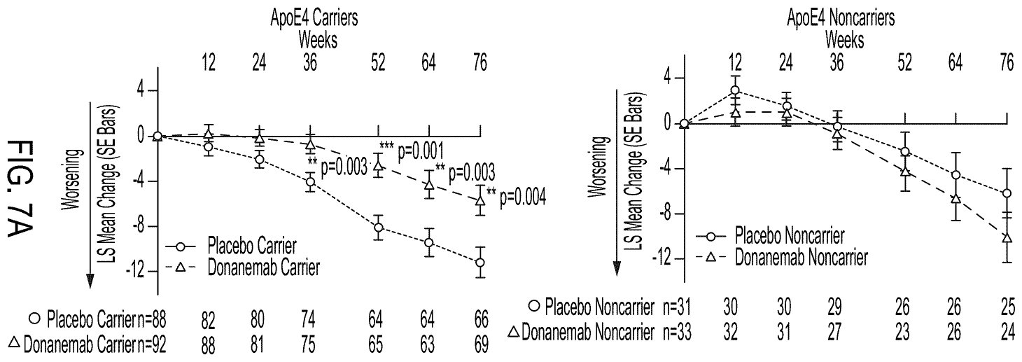

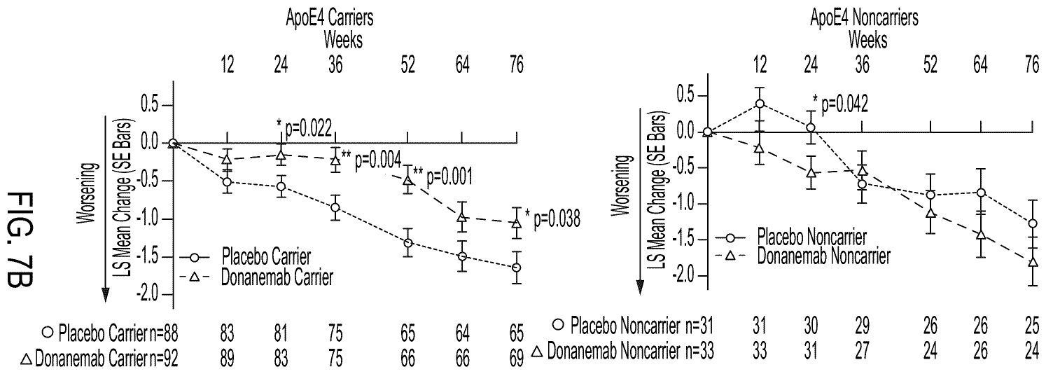

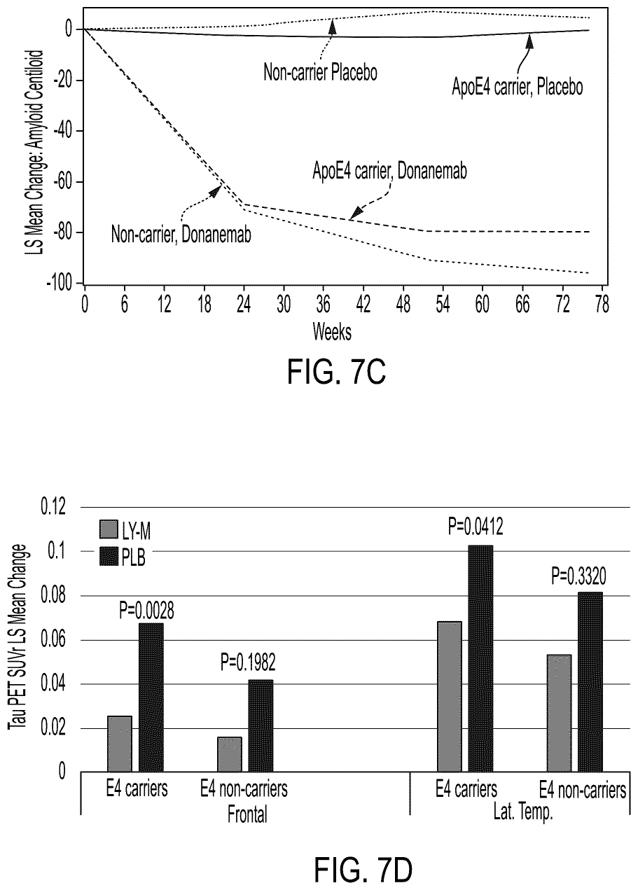

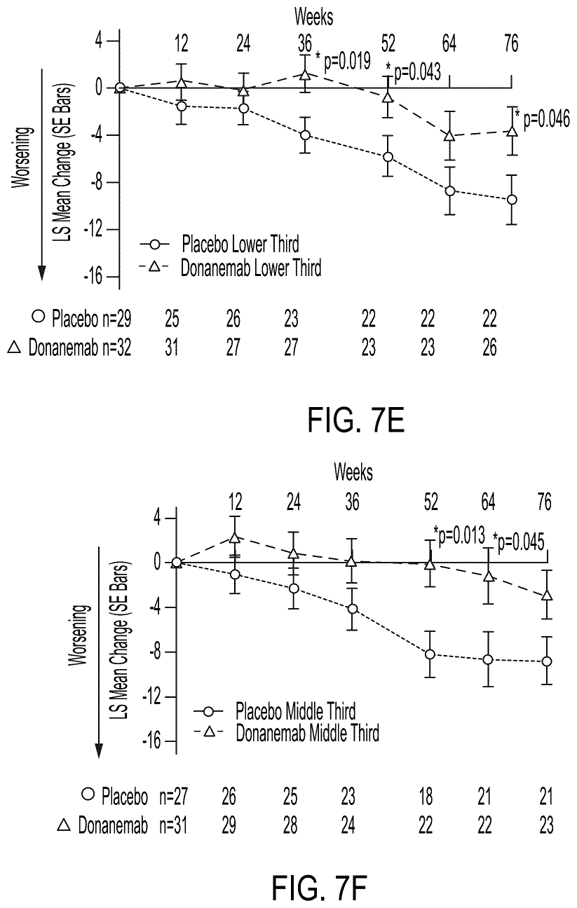

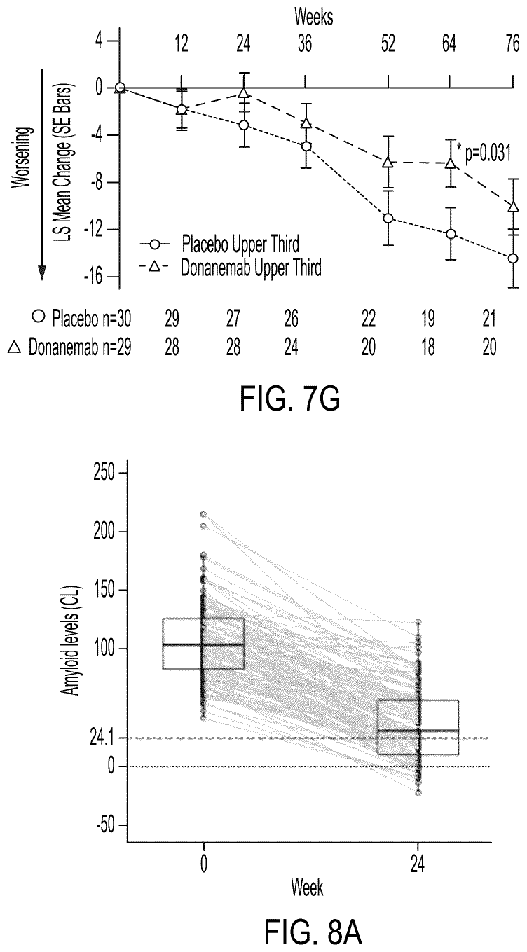

Results: Compared to placebo, donanemab treatment resulted in a 49% slowing of cognitive decline as measured with the iADRS (p=0.004) (FIG. 7A), and a 36% slowing of cognitive decline in the CDR-SB (p=0.038) in APOE4 carriers at 76 weeks (FIG. 7B).

The donanemab treatment differences between carriers and non-carriers was significantly greater for carriers (iADRS: p=0.001; CDR-SB: p=0.046). Additional key secondary endpoints showed a consistent and strong efficacy of donanemab compared to placebo in APOE4 carriers. See Table G and H below.

TABLE G
Secondary Endpoints for APOE4 Carriers
APOE4 Carriers

| | Treatment | | |
|---|---|---|---|
| Scale | Difference | % Slowing | P-Value |
| iADRS | 5.49 | 49.0 | 0.004 |
| CDR-SB | −0.59 | 36.0 | 0.038 |
| ADAS-Cog13 | −2.76 | 53.8 | 0.011 |
| ADCS-iADL | 2.61 | 44.1 | 0.030 |
| MMSE | 0.75 | 25.3 | 0.230 |

TABLE H
Secondary Endpoints for APOE4 Non-carriers
APOE4 Non-carriers

| | Treatment | | |
|---|---|---|---|
| Scale | Difference | % Slowing | P-Value |
| iADRS | −3.92 | −38.8 | 0.203 |
| CDR-SB | 0.52 | −28.9 | 0.270 |
| ADAS-Cog13 | 0.73 | −16.9 | 0.679 |
| ADCS-iADL | −3.11 | −53.9 | 0.116 |
| MMSE | 0.04 | 1.4 | 0.968 |

The safety profile for APOE4 carriers was consistent with the overall donanemab treatment population. Slowing of tau PET increases after treatment with donanemab was numerically larger in APOE4 carriers dosed with donanemab than non-carriers.

Amyloid-related imaging abnormalities (ARIA) with edema or effusions, most asymptomatic, were more common in APOE4 carriers (33.7%) than in noncarriers (8.3%). ARIA with hemosiderin deposits, like microhemorrhages, occurred in 34.5% of APOE4 carriers receiving donanemab. Censoring carrier subjects with ARIA did not change the significance of the placebo treatment difference for iADRS (p=0.020) and CDR-SB (p=0.050).

Analyses of the study population demonstrated higher efficacy of donanemab in APOE4 carriers than noncarriers, with significant slowing of disease progression measured in the both iADRS and CDR-SB.

FIGS. 7A-B show that donanemab exhibited higher efficacy in APOE4 carriers than non-carriers. FIG. 7A shows that donanemab exhibited higher efficacy in APOE4 carriers than non-carriers on iADRS scale. FIG. 7B shows that donanemab exhibited higher efficacy in APOE4 carriers than non-carriers on CDR-SB scale. FIG. 7C shows amyloid changes (centiloids) by APOE4 status of the patients in dosed and placebo arms. FIG. 7D shows change in Tau PET SUVR by APOE4 status of the patient. The left graph shows the frontal brain lobe data for carriers (referred to in the figure as E4 carriers) and non-carriers (referred to in the figure as E4 non-carriers) for APOE4. The right graph shows the lateral temporal brain lobe data for carriers (referred to in the Figure as E4 carriers) and non-carriers (referred to in the Figure as E4 non-carriers) for APOE4. FIGS. 7E-G show baseline tau subgroup analysis based on iADRS for APOE4 carriers in both the donanemab treated arm and the placebo arm. The lower third shows patients with baseline F18-flortaucipir (FTP) SUVR≤1.144 for both placebo and donanemab arms. The middle third shows patients with baseline FTP SUVR from 1.144 to 1.268 for both placebo and donanemab arms. The upper third shows patients with baseline FTP SUVR>1.268 for both placebo and donanemab arms.

### Example 6. Dynamics of Amyloid Reduction After Donanemab Treatment

Donanemab treatment resulted in rapid 24-week amyloid reduction and the rate of reduction was directly proportional to the amount of baseline amyloid. After 6 months of donanemab treatment participants with greater plaque removal showed less tau progression in frontal, parietal, and temporal brain regions and greater amyloid plaque changes associated with less cognitive decline.

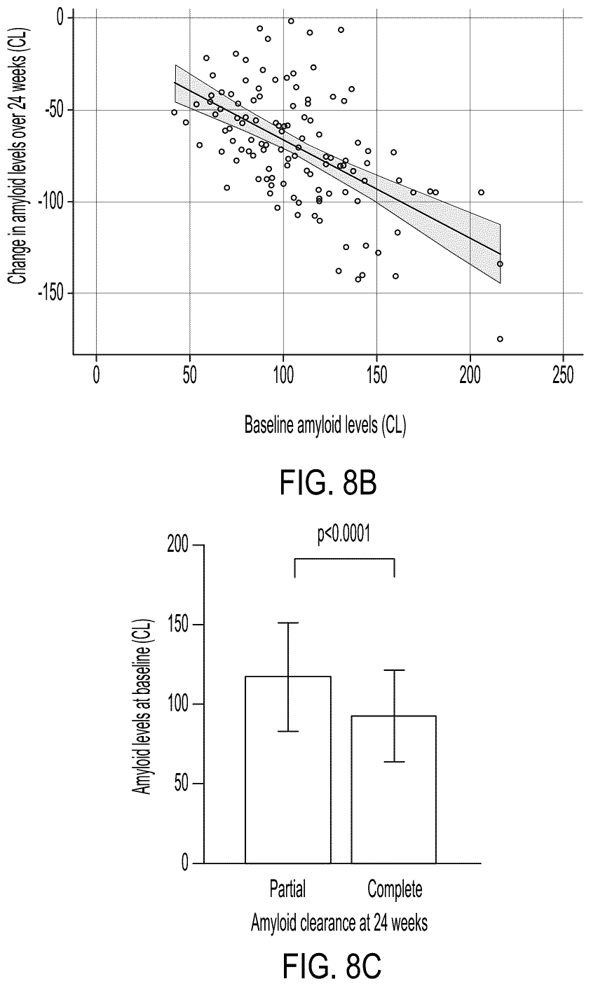

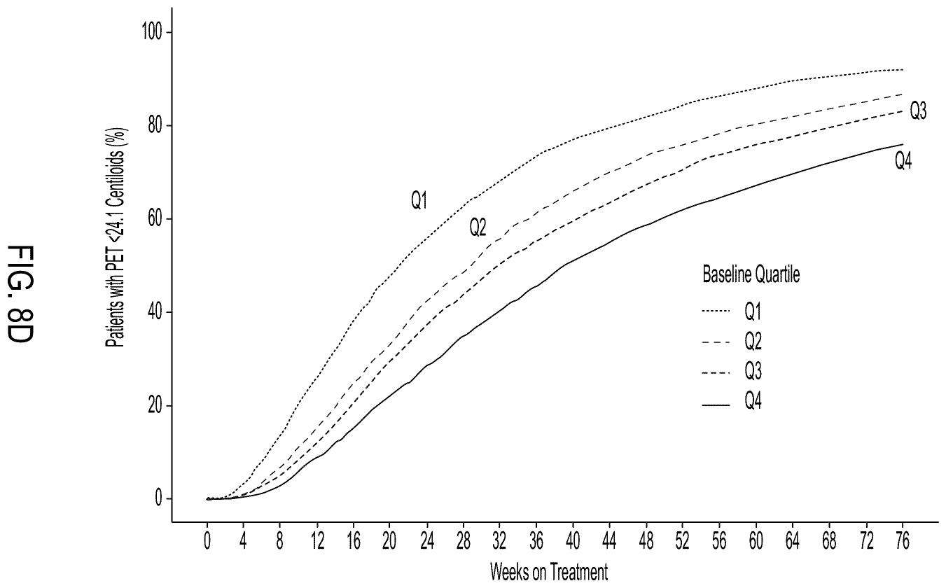

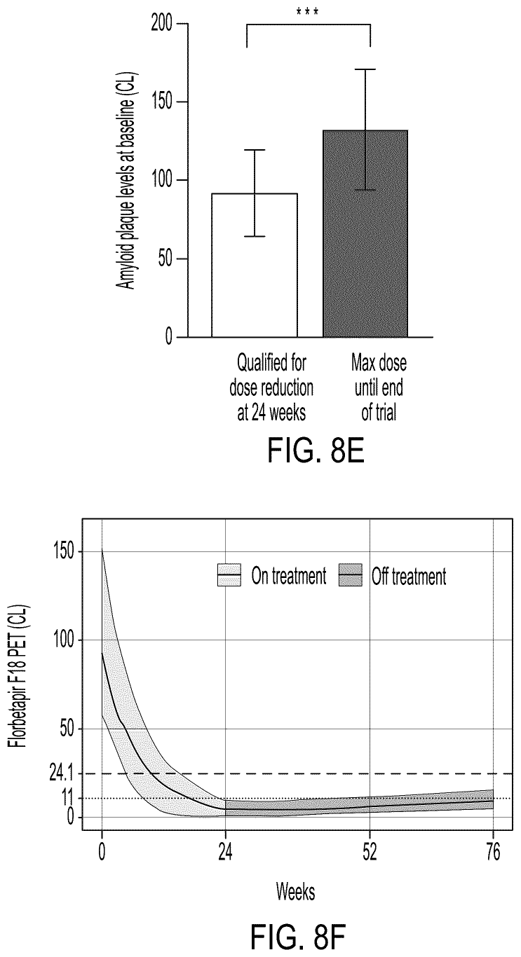

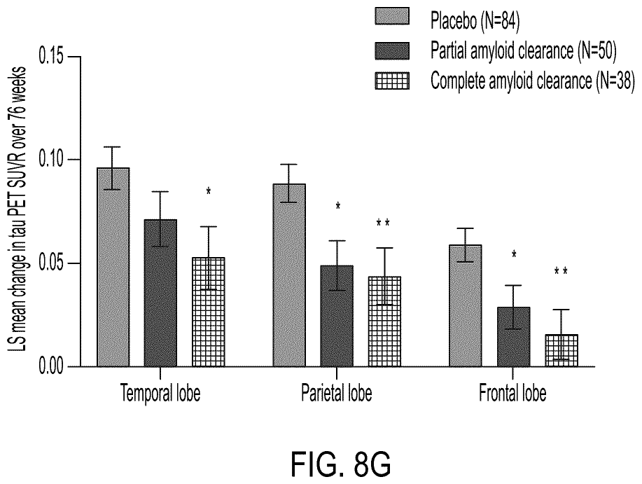

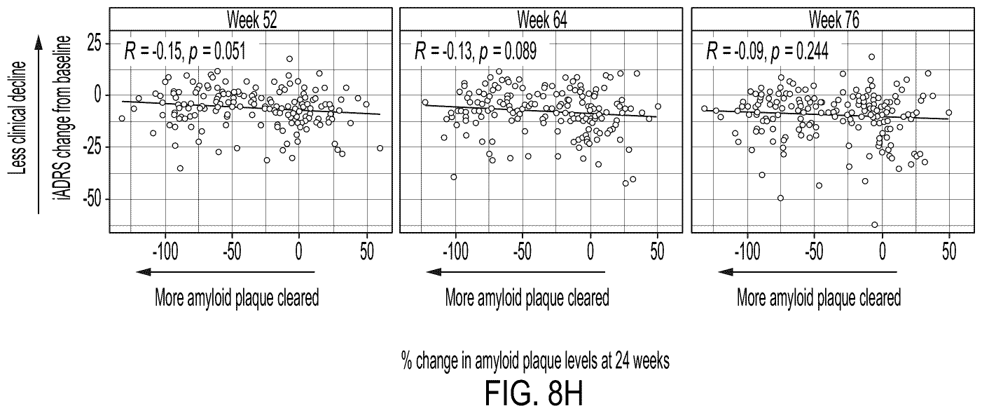

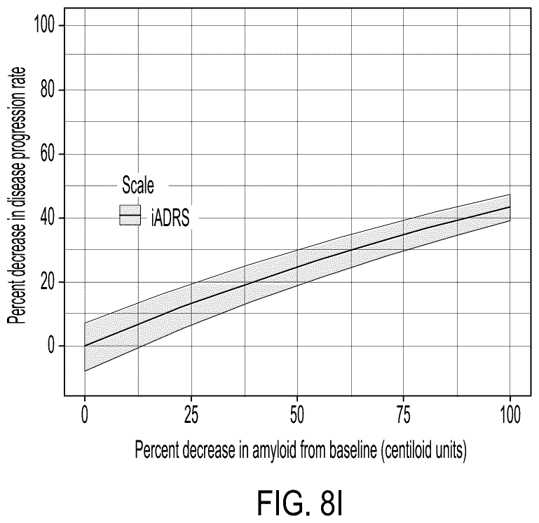

FIG. 8A shows that donanemab induced a rapid amyloid reduction in patients. The figure shows individual 24-week amyloid reduction trajectories for patients treated with donanemab.

Individual amyloid trajectories, shown in FIG. 8A, are based on the baseline and 24-week amyloid measurements (centiloid units, CL) observed in the clinical study TRAILBLAZER-ALZ (AACG, Clinicaltrials.gov identifier NCT03367403). Participants (N=115) were treated with donanemab and completed both baseline and 24-week F18-florbetapir PET scans. Complete amyloid removal (also referred to herein as amyloid negative and shown in FIG. 8A as dashed line) is defined as an amyloid plaque level of <24.1 CL (Mintun et al., “Donanemab in Early Alzheimer's Disease,” New England Journal of Medicine 384(18) (2021): 1691-1704, 2021, which is hereby incorporated by reference in its entirety). Donanemab induced a rapid and significant amyloid plaque reduction. All participants demonstrated amyloid reduction ranged from −1.8 CL to −174.8 CL. Mean amyloid reduction rate across all participants was −2.9 CL/week. The group average mean approached the complete amyloid removal threshold of 24.1 CL in the first 24 weeks. Individual trajectories also imply that individuals with a greater baseline amyloid plaque level are further from complete amyloid removal in the first 24 weeks of treatment, as illustrated by upper points on the plot. Conversely, participants with lower baseline amyloid plaque levels are closer to complete amyloid removal in the first 24 weeks of treatment, as illustrated by lower points on the plot.

FIG. 8B shows the association between baseline amyloid level (X-axis) and change in amyloid levels over 24 weeks (Y-axis) for participants treated with donanemab in TRAILBLAZER-ALZ. Amyloid plaque reduction is associated with baseline amyloid plaque level.

The relationship between baseline amyloid level and change in amyloid levels over 24 weeks of treatment with donanemab, shown in FIG. 8B, is based on the baseline and 24-week amyloid measurements (centiloid units, CL) observed in the clinical study TRAILBLAZER-ALZ (AACG, Clinicaltrials.gov identifier NCT03367403). Participants (N=115) were treated with donanemab and underwent both baseline and 24-week F18-florbetapir PET scans in this analysis. A robust correlation (Pearson correlation coefficient r=−0.57, p<0.001) with the total amyloid plaque level at baseline and the total amount of plaque removed in the first 24 weeks was observed. A greater baseline amyloid plaque level resulted in more amyloid plaque removal. Conversely, with lower baseline amyloid plaque levels, less plaque was removed.

Lower amyloid plaque level at baseline leads, on average, to earlier complete amyloid removal. FIG. 8C shows association between baseline amyloid level (Y-axis) and the amyloid removal reached at 24 weeks (X-axis) for participants treated with donanemab in TRAILBLAZER-ALZ. Participants with complete amyloid removal at 24 weeks had lower baseline amyloid plaque levels. In FIG. 8C, bars show mean+/−standard deviation; CL=Centiloids; PET=Positron Emission Tomography; Q=quartile.

The relationship between baseline amyloid level and the amyloid clearance level (partial or complete) obtained at 24 weeks, shown in FIG. 8C, is based on the baseline and 24-week amyloid measurements observed in the clinical study TRAILBLAZER-ALZ. Participants (N=115) were treated with donanemab and underwent both baseline and 24-week florbetapir PET scans and were included in this analysis. Patients were divided into two groups by the 24-week amyloid plaque level. Complete amyloid removal (also referred to herein as amyloid negative) is defined as an amyloid plaque level of <24.1 CL; partial amyloid removal is defined as an amyloid plaque level of ≥24.1 CL. Two-sample t-test is used to compare two groups. Participants who reached complete amyloid removal at 24 weeks had on average significantly (p<0.0001) lower baseline amyloid plaque levels than those who had partial amyloid removal at 24 weeks.

It was observed that participants with lower amyloid plaque baseline levels achieved complete amyloid removal faster (FIG. 8D). FIG. 8D shows modeled relationship of time to achieve plaque removal as a function of baseline amyloid plaque level. FIG. 8D represents the time to achieve complete amyloid plaque removal (defined as a PET measurement of <24.1 CL) in patients with varying levels of amyloid deposition at baseline. Simulations, shown in FIG. 8D, were conducted using an exposure-response model developed with data from clinical studies TRAILBLAZER-ALZ (AACG, Clinicaltrials.gov identifier NCT03367403) and AACD (Clinicaltrials.gov identifier NCT02624778). The model is an indirect-response model, with donanemab activity modeled as increasing the elimination rate constant associated with amyloid plaque level. To conduct the simulation, 10000 virtual patients were simulated to receive 3 doses of 700 mg donanemab IV, spaced 4 weeks apart, followed by 1400 mg donanemab Q4W for 17 doses, as in the dosing regimen used in TRAILBLAZER-ALZ. Patients were divided into quartiles (Q1-Q4) by baseline amyloid β plaque load (CL) value where Q1 is 38.7-81.6 centiloids; Q2 is 81.6-100.3 centiloids, Q3=100.3-126.3 centiloids, Q4 is 126.4-251.4 centiloids. At the end of 76 weeks of treatment, the model-estimated percentage of patients achieving amyloid removal (by quartile) was 92.1% (Q1), 86.8% (Q2), 83.1% (Q3), and 76.0% (Q4).

The analysis in FIG. 8D shows that patients with lower baseline amyloid levels were more likely to achieve amyloid clearance within 76 weeks of treatment than those who start therapy with higher baseline amyloid levels. For example, 92.1% patients in Q1 achieved complete amyloid clearance whereas 76.0% patients achieved amyloid clearance in Q4. Lower amyloid baseline patients appear to achieve plaque removal more quickly than higher amyloid baseline patients, as the time required for 50% of patients in each quartile to achieve amyloid removal corresponds with the relative amount of amyloid at baseline in each quartile.

The association between baseline amyloid level and donanemab dosing regimen is illustrated in FIG. 8E. The figure shows association between baseline amyloid level (Y-axis) and the donanemab dosing used. Participants with lower amyloid plaque baseline levels qualified for earlier dose reduction. In FIG. 8E, bars show mean+/−standard deviation; CL=Centiloids; Max=maximum; PET=Positron Emission Tomography; *** p<0.001.

In participants who were treated with donanemab, if the amyloid plaque level (as assessed by F18-florbetapir PET performed at 24 and 52 weeks) is 11 CL to less than 25 CL, indicating removal of amyloid plaques, the dose was lowered to 700 mg. If the amyloid plaque level was less than 11 CL on any individual scan or was 11 CL to less than 25 CL on two consecutive scans, donanemab-treated participants were switched to placebo. Two sub-groups are included in FIG. 8E: participants who stayed on the maximum dose until end of trial and participants who qualified for dose reduction at 24 weeks. Two-sample t-test was used to compare two groups. Participants meeting dose change criteria displayed significantly lower baseline amyloid plaque levels than those who remained on maximum treatment until the end of the trial.

Response rates to treatment with donanemab was dependent on baseline amyloid plaque levels and stopping dosing treatment does not result in significant amyloid reaccumulation over 1 year. FIG. 8F shows a model-predicted change in amyloid plaque levels after cessation of treatment in patients who achieved amyloid clearance within 6 months.

Using the model described above in FIG. 8F, the change in amyloid plaque levels was simulated in 2000 patients using the dosing regimen utilized in TRAILBLAZER-ALZ. Of these 2000 patients, the subset who achieved amyloid plaque levels of ≤11 CL are examined graphically to evaluate the projected time-course of amyloid plaque levels over the remainder of the trial. A value of 11 CL was used as the cut-off for this simulation, as this was the criteria used in TRAILBLAZER-ALZ for discontinuing donanemab treatment. Median (solid line) and 90% prediction intervals (shaded region) are plotted for the on- and off-treatment periods.

The impact of withdrawing treatment after a patient achieved <11 CL on plaque re-accumulation was investigated by simulations using a treatment exposure-response model (FIG. 8F). In a group of patients simulated to achieve PET signal<11 CL by week 24, withdrawal of donanemab treatment did not result in a substantial increase in PET signal through the end of the simulation (week 76), likely due to the extremely low rate of plaque accumulation estimated by the model (approximately 6.7 CL/year). The assumption of the model is that the rate of plaque formation/accumulation following donanemab treatment is similar to that at baseline. The implication of the model is that there is limited additional benefit to continued donanemab administration after complete amyloid clearance is achieved, as the relatively low rate of amyloid accumulation (6.7 CL/year) suggests that a patient achieving 11 CL while on donanemab would require more than 13 years to return to the model estimated baseline of 101 CL.

Donanemab treatment led to decreased tau accumulation over 76 weeks with less tau accumulation in participants with complete amyloid plaque clearance at 24 weeks. FIG. 8G shows the impact on tau PET for those reaching complete amyloid plaque removal at 24 weeks, compared to participants with partial amyloid removal or on placebo. TRAILBLAZER-ALZ study participants who had [¹⁸F] flortaucipir PET scans at baseline and 76 weeks are included in the analysis. Participants receiving donanemab (green bars in FIG. 8G) are designated as having partial or complete amyloid removal based on the amyloid plaque level at 24 weeks. Complete amyloid removal is defined as an amyloid plaque level of <24.1 CL, and the partial amyloid removal cohort included any donanemab-treated participants that did not reach that threshold by 24 weeks. Tau PET accumulation is measured by F18-flortaucipir regional SUVR in temporal, parietal, and frontal brain regions, using cerebellar-crus as the reference region. P-values indicate statistical significance versus placebo regional tau PET changes over 76 weeks (grey). In FIG. 8G, bars show mean+/−standard error; LS=least square; PET=Positron Emission Tomography; SUVR=Standardized Uptake Value Ratio; *p<0.05; **p<0.01 vs. placebo.

Less accumulation of aggregated tau as measured by F18-flortaucipir PET was observed at week 76 across temporal, parietal, and frontal brain regions in TRAILBLAZER-ALZ trial participants treated with donanemab. There is a numerically greater effect on tau changes (even less accumulation) for those participants that achieve complete amyloid clearance at 24 weeks in the study. These data highlight the value of rapid amyloid plaque clearance and support a model of amyloid-induced tauopathy and the connection of these biomarkers to the onset and/or progression of Alzheimer's disease.

FIG. 8H shows percent change in amyloid plaque levels at 24 weeks vs. iADRS change from baseline. Greater amyloid clearance at 24 weeks was associated with less clinical decline.

The percent change from baseline in CL value was calculated at week 24 for each patient and plotted against the change from baseline in iADRS (indicating a decrease in clinical disease progression) at weeks 52, 64, and 76 TRAILBLAZER-ALZ (FIG. 8H). Both donanemab and placebo treated patients were included in the plot. A simple linear regression line was fit to demonstrate the relationship between plaque reduction at week 24 and the clinical outcome of iADRS, and the Pearson correlation coefficient was calculated. A negative correlation coefficient is indicative of a linear relationship between more amyloid plaque removed and less clinical decline. At the timepoints of week 52, 64, and 76, the correlation coefficients were −0.15, −0.13, and −0.09, respectively. This analysis provides modest correlations which suggest that more amyloid plaque removed is associated with less clinical decline.

FIG. 8I shows the relationship between decrease in amyloid plaque and slowing in disease progression rate using a model integrating PK, PET, and clinical endpoint (iADRS) data. In the figure, iADRS=Integrated Alzheimer's Disease Rating Scale; mean and 90% CI; CI=Confidence interval; PET=Positron Emission Tomography; PK=pharmacokinetics.

A model was developed to describe the relationship between change in amyloid plaque levels with the change in the rate of disease progression as measured by the iADRS scale. This model is based upon a disease progression model described by Conrado et al., “An Updated Alzheimer's Disease Progression Model: Incorporating Non-linearity, Beta Regression, and a Third-level Random Effect in NONMEM,” Journal of Pharmacokinetics and Pharmacodynamics 41(6) 581-598, 2014, which is hereby incorporated by reference in its entirety. For this study, the Conrado model was modified to include a drug effect, which is modeled as attenuating the slope of the disease progression correlating to the percent change in amyloid plaque level from baseline as predicted by the exposure-response model for donanemab and amyloid plaque. FIG. 8I was generated using the model estimated slope of disease progression in the TRAILBLAZER-ALZ population, along with the model-estimated effect of amyloid plaque reduction on the disease slope. The 90% confidence interval for the relationship was estimated using the respective standard errors of the model parameters. The model predicted relationship was plotted as a solid line, and the 90% confidence interval was represented by a shaded region in FIG. 8I.

FIG. 8I demonstrates the modeled relationship between change in amyloid due to donanemab treatment with the change in rate of disease progression relative to placebo patients. This relationship is based on an exposure-response model linking serum donanemab concentrations with changes in amyloid levels, and, as a result of the change in amyloid level, a subsequent change in disease progression. The model suggests that complete removal of amyloid plaque may equate to a >40% reduction in the rate of disease progression. The model suggests a continuous relationship between reduction in amyloid plaque level and change in the disease progression rate. The continuous nature of the relationship suggests that even less than complete plaque removal will slow the rate of disease progression for patients, increasing the duration over which patients may maintain sufficient cognitive and functional activity to allow them to maintain an independent lifestyle.

### Example 7: Amyloid Clearance Results in Rapid and Sustained Reduction in Plasma Levels of Human Tau Phosphorylated at Threonine 217 (P-tau 217)

Amyloid clearance in subjects resulted in rapid and sustained reduction in plasma P-tau217 levels. The plasma P-tau217 was correlated with baseline amyloid plaque levels measured with F18-florbetapir PET and the baseline neurofibrillary tangles measured with F18-flortaucipir PET. Treatment with donanemab drives a rapid reduction of plasma P-tau217 detected within 12 weeks. Change in plasma P-tau217 was positively correlated with reduction in amyloid plaque by PET, slowing of tau neurofibrillary tangle growth by PET, and slowing of clinical progression as shown by the Conrado model. Moreover, similar to trends observed with slowing regional tau-PET, early complete amyloid plaque clearance suggests greater reduction in plasma P-tau217.

An immunoassay directed against human tau phosphorylated at threonine at residue 217 (P-tau217) was used to measure tau load/burden in human K2EDTA plasma for patients in TRAILBLAZER-ALZ (see, e.g., International Patent Application Publication No. WO 2020/242963, which is hereby incorporated by reference in its entirety). The anti-tau antibodies disclosed in WO 2020/242963 are directed against isoforms of human tau expressed in the CNS (e.g., recognizing the isoforms expressed in the CNS and not recognizing isoforms of human tau expressed exclusively outside the CNS).

A Quanterix Simoa® HD-X Analyzer™ was used for the p-Tau 217 immunoassay. The analyzer uses P-tau217 immunoassay reagents (capture antibody: Fab clone against P-tau217; detection antibody: antibody clone against tau protein; calibrator and control: two synthetic peptides coupled with a PEG linker representing the epitopes recognized by capture and detection antibodies. See, e.g., International Patent Application Publication No. WO 2020/242963, which is hereby incorporated by reference in its entirety) using Single Molecule Array (Simoa®) technology. This assay may detect low levels of P-tau217 in human plasma and is a fully automated immunoassay.

In a first step, target antibody-coated capture beads were combined with human plasma sample. Target molecules present in the sample were captured by the antibody coated capture beads. After washing, biotinylated detector antibodies were mixed with the capture beads. The detector antibodies bind to the captured target. Following a second wash, a conjugate of streptavidin-β-galactosidase (SBG) was mixed with the capture beads. SBG binds to the biotinylated detector antibodies, resulting in enzyme labeling of captured target. Following a third wash, the capture beads were resuspended in a resorufin β-D-galactopyranoside (RGP) substrate solution and transferred to a Simoa® Disc. Individual capture beads were then sealed within microwells in the array. If target was captured and labeled, the β-galactosidase hydrolyzed the RGP substrate into a fluorescent product that provided the signal for measurement. A single-labeled target molecule results in a sufficient fluorescent signal to be detected and counted in 30 seconds by the Simoa® optical system. At low target concentration, the percentage of bead-containing wells in the array that have a positive signal is proportional to the amount of target present in the sample. At a higher target concentration, when most of the bead-containing wells have one or more labeled target molecules, the total fluorescence signal is proportional to the amount of target present in the sample. The concentration of target in unknown samples is interpolated from the calibration curve using a log-log power regression without weighting.

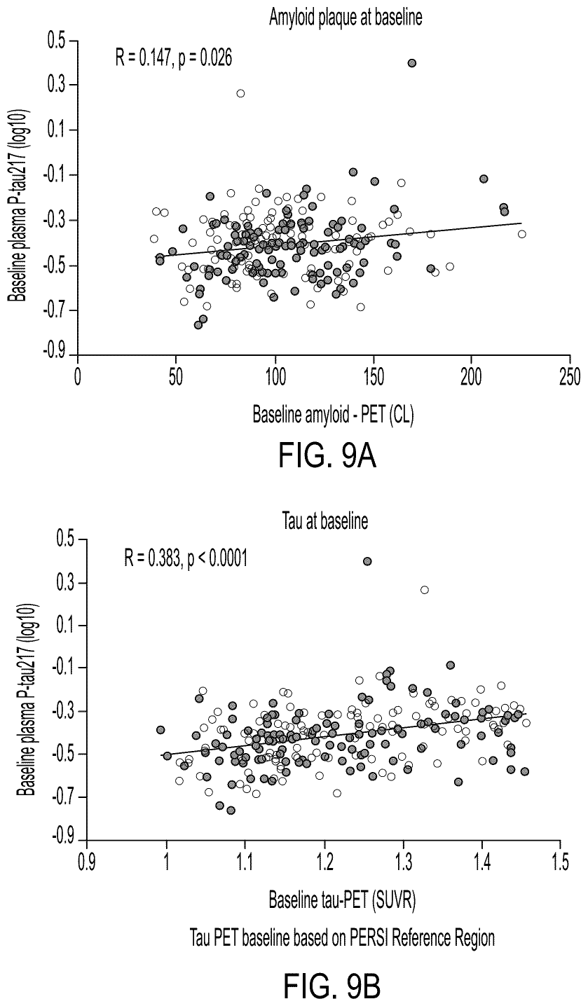

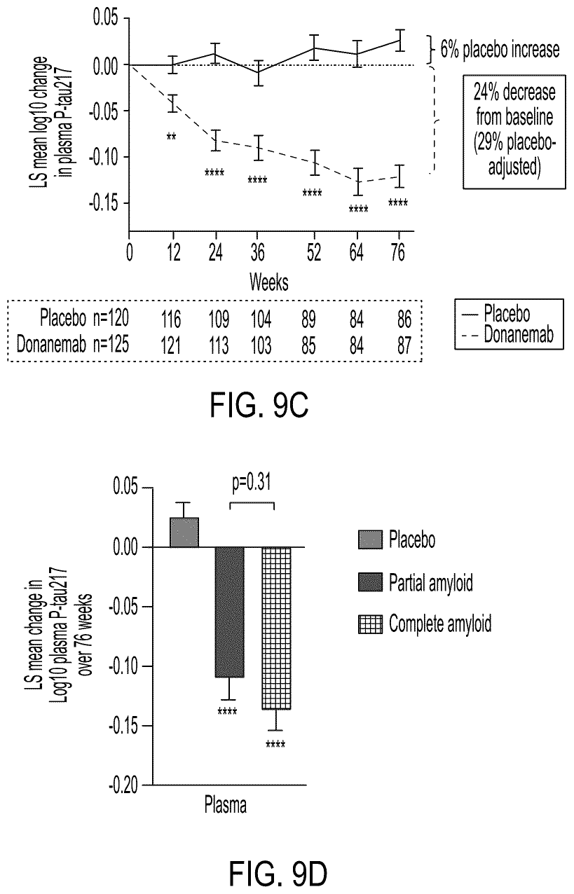

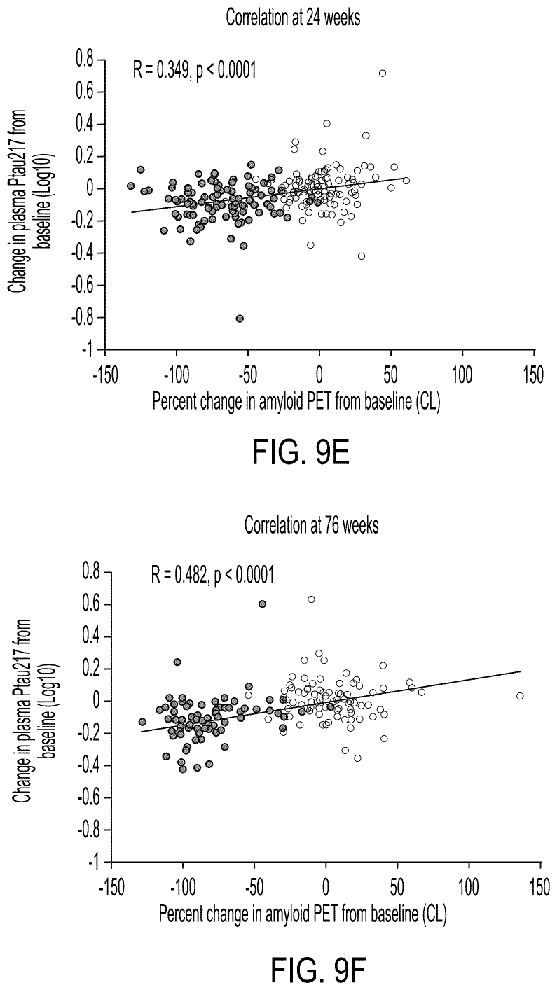

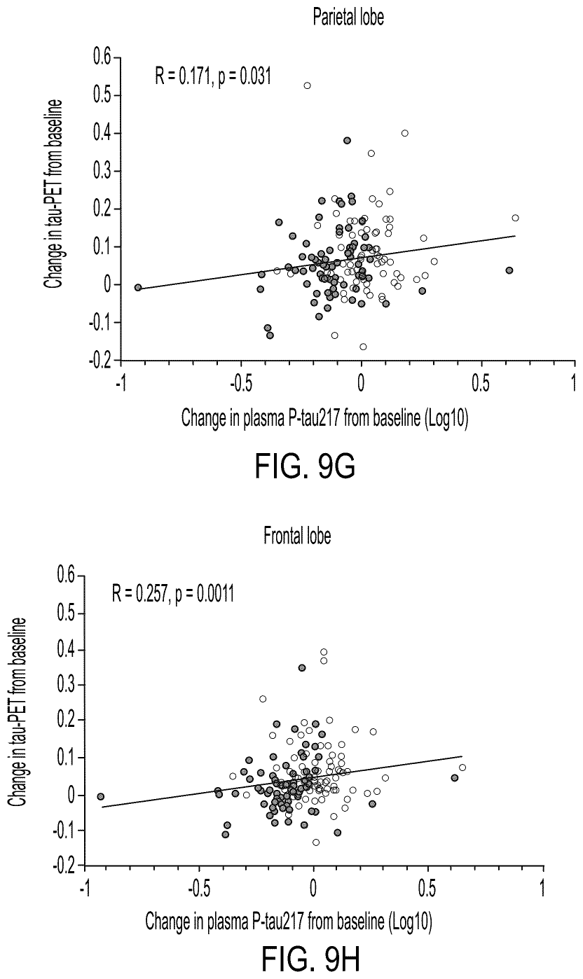

FIGS. 9A-B show that the baseline plasma P-tau217 is correlated with baselines amyloid plaque level and neurofibrillary tangles. FIG. 9A shows a scatter plot of baseline amyloid PET centiloid and baseline plasma P-tau217. The hollow circles depict patients in TRAILBLAZER-ALZ on placebo and green solid circles show patients in TRAILBLAZER-ALZ who received donanemab. P-tau217 values were normalized by log 10 transformation. Correlation between the two variables was assessed using Spearman's rank correlation. At baseline, β-amyloid as measured by F18-florbetapir PET showed a positive correlation with plasma P-tau217 level (R=0.147, p=0.026). FIG. 9B shows a scatter plot of baseline tau PET MUBADA SUVR and baseline plasma P-tau217. The hollow circles depict patients in TRAILBLAZER-ALZ on placebo and green solid circles show patients in TRAILBLAZER-ALZ who received donanemab. P-tau217 values were normalized by log 10 transformation. Correlation between the two variables was assessed using Spearman's rank correlation. At baseline, brain tau as measured by F18-florbetapir PET showed a positive correlation with plasma P-tau217 level (R=0.383, p<0.0001). In FIGS. 9A-B, CL=Centiloids; SUVR=Standardized Uptake Value Ratio; PET=Positron Emission Tomography; p=p-value; R=correlation coefficient; SUVR=Standardized Uptake Value Ratio.

The immunoassay data shows that plasma P-tau217 was significantly decreased with donanemab treatment in human subjects. FIG. 9C shows mixed model with repeated measures (MMRM) to compare the P-tau217 change from baseline between treatment arms. The figure shows that donanemab delivers early reduction plasma P-tau 217. FIG. 3A (provided above) shows that amyloid plaque is significantly lowered with donanemab treatment. P-tau217 showed a fast clearance post treatment, starting from 12 weeks measurement. At week 76, the donanemab treated group showed a 24% decrease comparing to baseline (P<0.0001) while the placebo treated group showed a 6% increase (p=0.03). Comparing to placebo-treated group, the donanemab treated group showed a 29% decline in terms of P-tau217 accumulation. In FIG. 9C, LS=Least Square; p=p-value, ** p<0.01; **** p<0.0001 vs placebo.

The change of plasma P-tau217 was associated with amyloid plaque removal status at 24 weeks. FIG. 9D shows mixed model with repeated measures (MMRM) to compare the P-tau217 change from baseline across placebo, donanemab treated with partial amyloid removal, and donanemab treated with complete amyloid removal groups. Complete amyloid removal was defined as a florbetapir PET centiloid level<24.1 (also referred to herein as amyloid negative). The amyloid clearance status was determined using F18-florbetapir PET scan at 24 weeks. In FIG. 9D, bars show mean+/−standard error, LS=least squares; p=p-value; ****p<0.0001 vs. placebo.

Consistent with the finding from brain tau PET, amyloid removal was associated with P-tau217 reduction. At 76 weeks, though statistically non-significant (p=0.34), complete amyloid removal at 24 weeks showed a numerically larger P-tau217 reduction than partial amyloid removal. Both treated groups showed a statistically significant decrease in P-tau217 accumulation comparing to placebo group (p<0.0001).

FIGS. 9E and 9F show that decreased plasma P-tau217 is associated with amyloid clearance. FIGS. 9E and 9F show a scatter plot of amyloid PET centiloid change from baseline with P-tau217 change from baseline values at 24 and 76 weeks, respectively. P-tau217 values were normalized by log 10 transformation. Correlations between the two sets of variables were assessed using Spearman's rank correlation. In the figures, hollow circles show patients in TRAILBLAZER-ALZ on placebo and green solid circles show patients in TRAILBLAZER-ALZ who received donanemab; CL=centiloids; PET=Positron Emission Tomography; p=p-value; r=correlation coefficient.

At both time points (24 weeks and 76 weeks), β-amyloid change from baseline values, as measured by F18-florbetapir PET, showed positive correlations with P-tau217 level (r=0.349 and 0.482 respectively. p<0.001 for both correlation coefficients).

Decreased plasma P-tau217 was associated with less neurofibrillary tangles at 76 weeks. FIGS. 9G and 9H show a scatter plot of tau PET regional SUVR (frontal and parietal) change from baseline with P-tau217 change from baseline values at 76 weeks. P-tau217 values were normalized by log 10 transformation. Correlations between the two sets of variables were assessed using Spearman's rank correlation. In the figures, hollow circles show patients in TRAILBLAZER-ALZ on placebo and solid circles show patients in TRAILBLAZER-ALZ who received donanemab; PET=Positron Emission Tomography; p=p-value; r=correlation coefficient.

Both frontal and parietal lobe SUVR change from baseline values show positive correlations with P-tau217 change from baseline values (r=0.171, p=0.031 and r=0.257, p=0.0011 respectively).

A PK/PD model demonstrates a relationship between plasma P-tau217 and slowing of clinical decline. This model was developed to describe the relationship between change in plasma P-tau217 levels with the change in the rate of disease progression as measured by the iADRS scale. This model was based upon a disease progression model described by Conrado et al (Conrado et al., “An Updated Alzheimer's Disease Progression Model: Incorporating Non-linearity, Beta Regression, and a Third-level Random Effect in NONMEM,” Journal of Pharmacokinetics and Pharmacodynamics 41(6) 581-598, 2014, which is hereby incorporated by reference in its entirety).

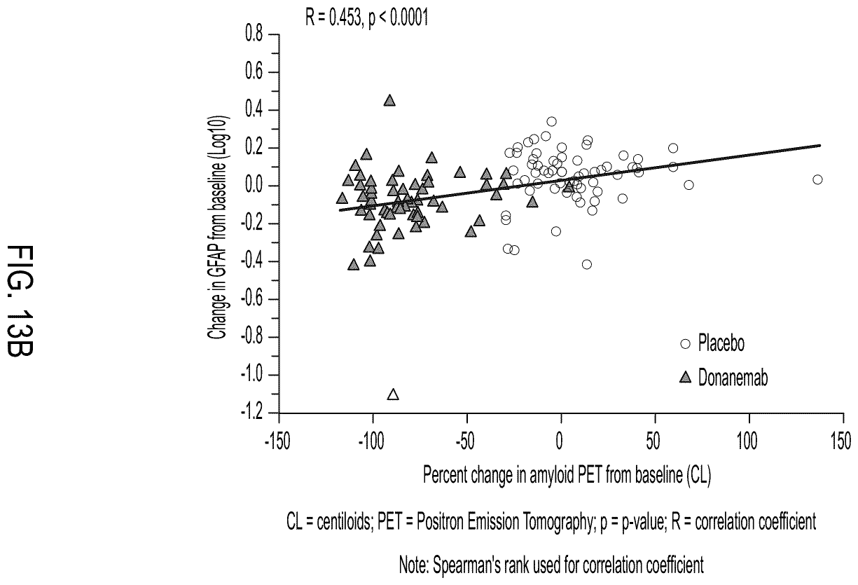

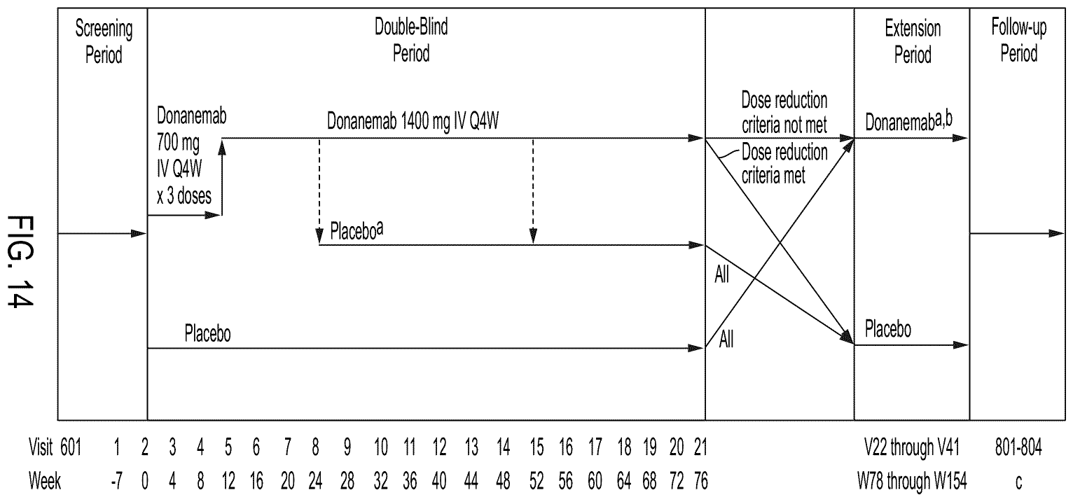

The model shows P-tau217 reduction is statistically significant (FIG. 91) as a predictor of slowing of clinical decline (p<0.001). In FIG. 91, iADRS=Integrated Alzheimer's Disease Rating Scale; mean and 90% CI; CI=Confidence interval; PK=pharmacokinetics; p=p-value.

### Example 8: TRAILBLAZER-ALZ 3 Trial Design and Rationale

Objectives: TRAILBLAZER-ALZ 3 (herein referred to as TB3, NCT05026866), is a multicenter, randomized, double-blind, placebo-controlled, event-driven Phase 3 trial of decentralized design with central raters designed to assess the impact of donanemab versus placebo in cognitively unimpaired participants with evidence of AD pathology (preclinical AD). FIG. 10 illustrates the trial design for the clinical protocol. SP stands for study period. If donanemab meets defined success factors, then participants who were randomized to placebo and complete SP III may have access to donanemab in SP IV, open label extension.
Overview and Primary Endpoints: Approximately 3300 participants who meet entry criteria will be randomized in a 1:1 ratio to either donanemab (700 mg intravenously (IV) once every 4 weeks (Q4W) for the first 3 doses, then 1400 mg IV Q4W for the next 6 doses) or placebo (IV Q4W for 9 doses). Participants will be followed until approximately 434 participants experience a primary outcome event of clinical progression (an increase at 2 consecutive visits in the Clinical Dementia Rating Global Score (CDR-GS or gCDR) from CDR-GS=0 at baseline), so the total duration of study participation will vary for each participant, with continued follow-up from 3-5 years. Treatment with donanemab will be stratified by APOE4 allelic dose of 0-2. For example, an individual may have 0, 1, or 2 copies of the E4 allele. Individuals are APOE4 negative if they have 0 copies of APOE4 allele, heterozygous if they have 1 copy of APOE4 allele, or homozygous if they have 2 copies of APOE4 allele. Such stratification by APOE4 dose allows for equal amounts of homozygous and heterozygous E4 carriers in each treatment arm, rather than classifying individuals by APOE4 carrier status.

This trial will use a Decentralized Clinical Trial (DCT) Model with visits conducted remotely in whole or in part, with a goal of increasing the number of eligible participants, including those from under-represented groups. All clinical and cognitive assessments will be conducted remotely by central raters. The DCT Model includes the use of technology, flexible locations and central staffing to optimize the likelihood of strong participant retention and increased standardization with centralized raters for clinical assessments. Participants and Partners are to be assigned a Central Study Coordinator (CSC) who will serve as their central contact throughout the study.

### Inclusion Exclusion Criteria:

### Inclusion Criteria Include:

Males & females aged 55-80 years old;
Telephone interview for cognitive status—Modified (TICS-m) score of intact cognitive functioning; and
Eligible plasma P-tau217 result (SIMOA assay).

### Exclusion Criteria Include:

Mild cognitive impairment (MCI)/dementia or other neurodegenerative disease affecting cognition;
Current or previous use of prescription medications for treatment for MCI or dementia;
Current serious or unstable illnesses;
History of cancer with high risk of recurrence and preventing completion of the trial;
History of clinically significant multiple or severe drug allergies, or severe post-treatment hypersensitivity reactions;
Prior treatment with an anti-amyloid immunotherapy;
Any clinically important abnormality at screening on MRI or clinical laboratory test
Any contraindications for MRI; and
A centrally read MRI demonstrating presence of ARIA-E (Amyloid-related imaging abnormalities with effusion or edema), >4 cerebral microhemorrhages, more than 1 area of superficial siderosis, any macrohemorrhage or severe white matter disease at screening.
   Other Efficacy Assessments: Secondary endpoints to assess clinical progression include International Shopping List Test, Continuous Paired Associate Learning, International Daily Symbol Substitution Test-Medicines, Category Fluency, Face Name Association Test, Behavioral Pattern Separation-Object test, Cogstate Brief Battery, CDR-Sum of Boxes, Cognitive Function Index, Montreal Cognitive Assessment, and Cognitive composite score (involving a combination of individual assessments). Optional addenda may include Florbetapir-18F PET scan, (N=200), Flortaucipir-18F PET scan (N=500), and APOE disclosure.
   Safety Assessments: To evaluate safety and tolerability of donanemab, this study will monitor spontaneously reported adverse events (AEs), MRI (for ARIA and emergent radiological findings), infusion-related reactions, and Columbia Suicide Severity Rating Scale. MRI to assess/monitor for ARIA may be conducted at baseline, after the first dose, before increasing the dose from 700 mg to 1400 mg, at 4 weeks, at 12 weeks, at 20 weeks, and every 1-2 weeks throughout the dosing period (e.g., double blind treatment period) or as determined by the investigator. MRI scheduling may be reinitiated during the open-label extension period to assess/monitor for ARIA.
   Biomarkers: Serum, plasma, and whole-blood RNA samples for biomarker research will be collected at screening and throughout the study. Biomarker analysis will be performed to address questions of relevance to drug disposition, target engagement, pharmacodynamics, mechanism of action, and variability of participant response (including safety). Plasma P-tau217 and other blood-based biomarkers will be used to further inform clinical outcomes and response to therapy. To assess the effect of donanemab on cerebral amyloid plaque burden and cerebral neurofibrillary tangle burden relative to placebo in the preclinical AD population, a subset of participants will undergo florbetapir and/or flortaucipir PET imaging.
   Potential Impact Conclusions: TB3 represents an innovative decentralized trial design with central raters. It includes a time-to-clinical-event model, a blood-based AD biomarker selection criterion, and potentially supportive AD biomarker endpoints. The results of this trial may help address the question of whether donanemab treatment with rapid lowering of cerebral amyloid plaque can delay or even prevent progression to the clinical stages of AD.

### Example 9: Biomarkers for TRAILBLAZER-ALZ

Additional biomarker data was generated using Simoa® Neurology 4-Plex E Advantage Kit (additional information about the assay/kit is provided at “quanterix.com/wp-content/uploads/2020/12/Neurology-4-Plex-E-Data-Sheet-HD-X.pdf” which is hereby incorporated by reference in its entirety). Briefly, the Simoa® Neuro 4-plex E assay is a digital immunoassay for quantitative determination of Amyloid Beta 40 (Aβ₄₀), Amyloid Beta 42 (Aβ42), Neurofilament Light Chain (NfL), and Glial Fibrillary Acidic Protein (GFAP) in human plasma. The Simoa® HD-X Analyzer™ uses ready-to-use Neuro 4-plex E immunoassay reagents to perform jobs (one job equals a single value, so duplicate runs are two jobs) using Single Molecule Array (SiMoA) technology. In the two-step Simoa® Neuro 4-plex E assay, target antibody-coated paramagnetic beads are combined with sample and biotinylated detector antibody in the same incubation. Target molecules present in the sample are captured by the antibody coated beads and bind with the biotinylated antibody detector simultaneously. Following a wash, a conjugate of streptavidin-β-galactosidase (SBG) is mixed with the beads. SBG binds to the biotinylated detector antibodies, resulting in enzyme labeling of the captured target. Following a final wash, the beads are resuspended in a resorufin β-D-galactopyranoside (RGP) substrate solution and transferred to the Simoa® Disc. Individual beads are then sealed within microwells in the array. If the target has been captured and labeled on the bead, β-galactosidase hydrolyzes the RGP substrate in the microwell into a fluorescent product that provides the signal for measurement. A single-labeled target molecule results in a sufficient fluorescent signal to be detected and counted in 30 seconds by the Simoa® optical system. At low target concentration, the percentage of bead-containing wells in the array that have a positive signal is proportional to the amount of target present in the sample. At a higher target concentration, when most of the bead-containing wells have one or more labeled target molecules, the total fluorescence signal is proportional to the amount of target present in the sample. The concentration of target in unknown samples is interpolated from the calibration curve using a four/five-parameter logistic regression with 1/y² weighting.

### Neurofilament Light Chain (NfL)

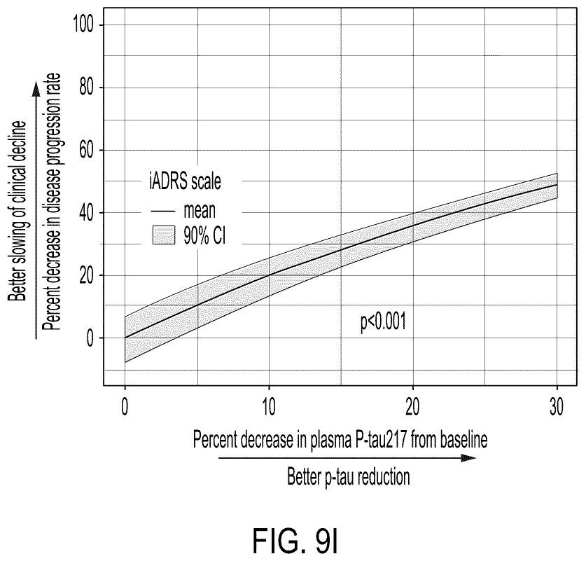

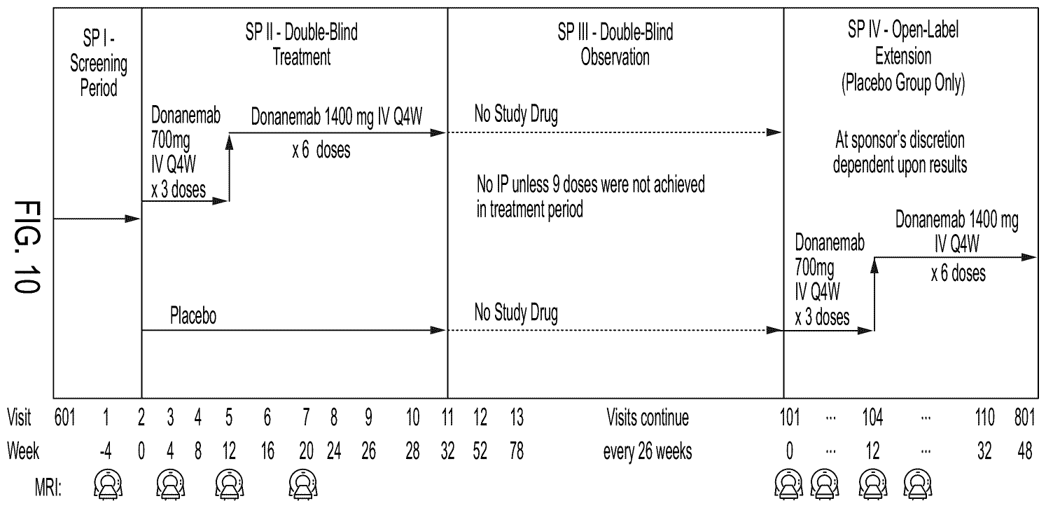

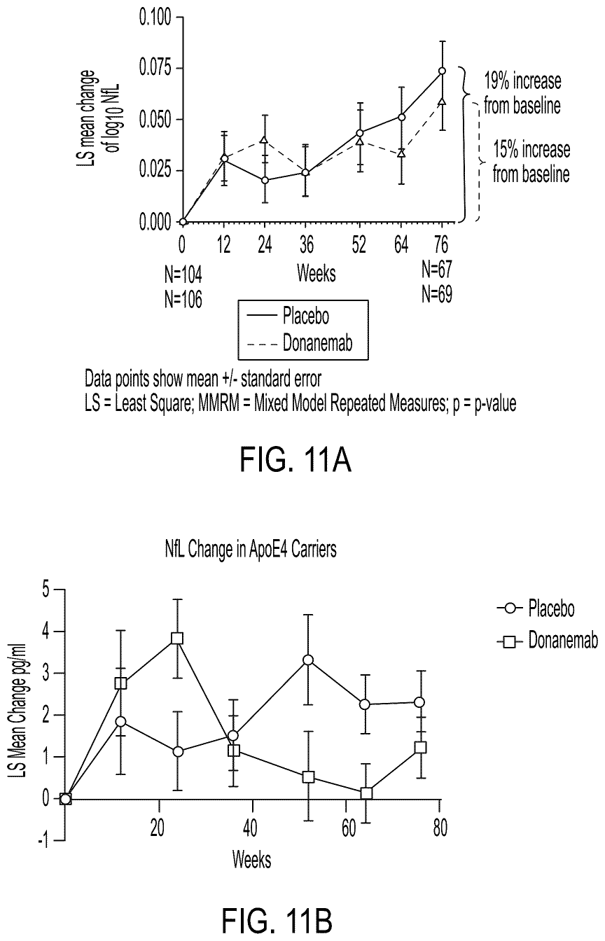

Neurofilament light chain (NfL) is an important biomarker (plasma or CSF) that can show neuronal injury through many disease mechanisms but in particular is elevated in Alzheimer's disease as well. Therapies that can reduce NfL are presumed to reduce neuronal injury and portend improved outcomes for that disease. The data obtained from the Simoa® Neuro 4-plex E assay demonstrates that NfL, in the whole donanemab treated population, can be reduced by at least 4% compared to placebo over the course of treatment/dosing regimen (FIG. 11A shows reduction in plasma Nf1). This reduction effect may be enhanced in APOE4 carriers as illustrated in FIG. 11B which shows significant lowering at time points later in the trial. The plasma data in the figure is the first demonstration of NfL reduction with an anti-Aβ antibody in Alzheimer's Disease in an APOE4 carrier population. FIG. 11B shows change from baseline in NfL in APOE4 carriers (LS-Mean Estimates from MMRM Model.

Amyloid Beta (Aβ)

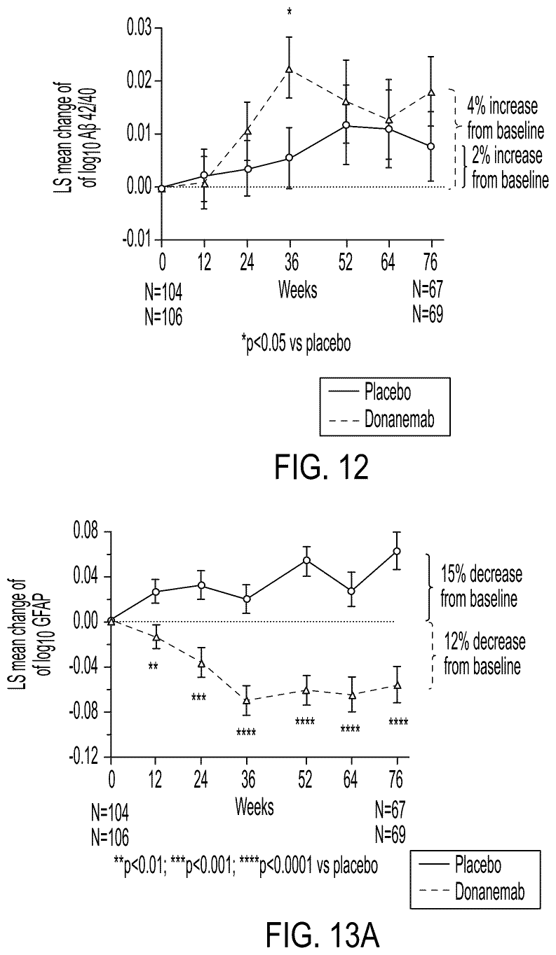

Aβ_{42/40} ratio is known to decrease slowly over time in plasma and CSF as amyloid plaque is deposited in the brain parenchyma. Therapies that remove amyloid plaque may normalize this ratio, which is interpreted as increasing back toward a higher level. Data obtained from TRAILBLAZER-ALZ shows that donanemab treatment can increase this ratio and show significant improvement in at least one timepoint (FIG. 12). FIG. 12 shows increase in Aβ42/40 Ratio.

It is notable that donanemab does not interact with any soluble species, unlike some other antibodies, so the elevation of this ratio provides a definitive result in the ability of amyloid plaque clearance to normalize this biomarker without any confounding effects previously demonstrated with other antibodies due to their binding of other soluble blood species.

### Glial Fibrillary Acidic Protein (GFAP)

The normalization and reduction of the GFAP biomarker is an important first for plaque lowering antibodies. GFAP is an intermediate cytoskeletal protein that is upregulated in reactive astrocytes and has been recognized as a pathological feature in many diseases but also well described in AD. GFAP is associated with amyloidosis and recent studies have linked GFAP in blood with amyloid deposition. The data shown in FIG. 13A shows for first time the ability of a therapeutic (donanemab) to reduce this pathological response of astrocytes to amyloid through a reduction of GFAP as measured in the blood. FIG. 13A shows GFAP is significantly lowered with donanemab treatment. Additionally, P-tau 217 and GFAP show similar relationship with amyloid plaque clearance in TRAILBLAZER-ALZ. FIG. 13B shows correlation between plaque clearance and GFAP at 76 weeks.

Other ways to document this reduction in pathology would theoretically exist but could include PET tracer like C11 deprenyl. Astrocytes play an important role in maintenance of the blood brain barrier through interaction of the capillaries via astrocytic end-plate connections. In addition, astrocytes play an important role in glutamate toxicity by regulating and assisting with the removal or uptake of glutamate in the synaptic cleft. The improvement of this biomarker portends a clinical benefit and an improvement of the amyloid induced pathology that develops over the course of amyloid deposition. The improvement of this biomarker may indicate improved blood-brain barrier function which is impaired in Alzheimer's disease and improvement in neuronal function or survival. The benefit of amyloid reduction and normalization of GFAP levels are not limited to these examples. Other examples may include improvement of health of astrocytes with survival of primary and secondary branches, scar formation, immune-cell recruitment, synaptic remodeling, metabolic regulation, circadian rhythm, calcium dynamics, neurotransmitter regulation, and antioxidant buffering. In addition, these data may suggest that GFAP may be an important therapeutic target for the treatment of AD.

### Example 10: Assessment of Safety, Tolerability, and Efficacy of Donanemab in Early Symptomatic Alzheimer's Disease

A multicenter, randomized, double-blind, placebo-controlled, Phase 3 clinical study (NCT04437511; clinicaltrials.gov; herein referred to as “TRATLBLAZER-ALZ 2,” “AACI,” or “Study AACI”) is designed to evaluate the safety and efficacy of a humanized N3pG Aβ antibody (donanemab) in patients with early symptomatic AD (i.e., prodromal AD and mild dementia due to AD) with the presence of brain tau pathology. AACI will assess whether removal of existing amyloid plaque can slow the progression of the disease as assessed by clinical outcomes for cognition and function, and by imaging biomarker measures of disease pathology and neurodegeneration over 76 weeks of double-blind observation.

AACI expands the patient population compared to the prior Phase 2 (i.e., TRAILBLAZER-ALZ, also known as AACG, Clinicaltrials.gov identifier NCT03367403)) study by including participants with high tau pathology. The primary analysis will test the low-medium tau pathology population and the overall population (low-medium and high tau pathology) and includes a long-term extension period designed to further evaluate donanemab efficacy and safety over time.

This study will assess, including other things, whether removal of existing amyloid plaque can slow the progression of disease as determined by clinical measures and biomarkers of disease pathology and neurodegeneration over up to 76 weeks of treatment. Participant randomization will be stratified by investigative site and tau pathology (low-medium versus high). This is a parallel, double-blind treatment study with 2 treatment groups. The study includes a screening visit, which can last up to 49 days, at which participants are required to have F18-flortaucipir PET tau imaging results consistent with elevated tau in order to be randomized to the double-blind period. The duration of the double-blind period of the study is 76 weeks and includes up to 72 weeks of treatment with endpoint measures at the end of the double-blind period (Week 76), to assess the safety, tolerability, and efficacy of donanemab versus placebo. Approximately 1800 participants will be randomized in the trial, across Cohorts 1 and 2. Cohort 1 will consist of no greater than approximately 300 randomized participants and will include participants randomized prior to a prespecified date. Approximately 1000 low-medium tau participants will be randomly assigned to donanemab in Cohort 2. It is anticipated that approximately 1500 participants overall (low, medium, and high tau pathology) will be randomized in the study to achieve approximately 1000 low-medium tau under the assumption that approximately one-third of participants have high tau pathology in Cohort 2.

Participants who meet entry criteria will be randomized in a 1:1 ratio to one of the following treatment groups: donanemab, 700 mg intravenously (IV) Q4W for first 3 doses and then 1400 mg IV Q4W; or placebo. The maximum total duration of study participation for each participant, including screening and the post-treatment follow-up periods, is up to 205 weeks, and includes a screening period of up to 7 weeks, randomization at visit 2, a double-blind treatment period (visits 2-21) of up to 72 weeks, final endpoint measures and safety assessments for the double-blind period performed at visit 21 (V21, week 76, 4 weeks following the participant's last dose of donanemab) with evaluation for amyloid plaque reduction by PET scan at V21, with a possible extension part of 78 weeks (maximum duration of treatment period with donanemab is 150 weeks), immunogenicity and safety follow-up visits beginning 12 weeks after their last dose of donanemab, and a follow-up period of up to 44 weeks. In the extension part of the study (visits 22-41), participants randomized to donanemab during the double-blind period who do not meet dose reduction criteria by V21 will continue receiving donanemab. Participants who remained on 700 mg during the double-blind period will have the opportunity to dose escalate to 1400 mg at V25 or after. Participants randomized to donanemab during the double-blind period who meet dose reduction criteria by V21 will be assigned to receive placebo starting at V22. Participants randomized to placebo during the double-blind period will be assigned to receive donanemab starting at V22 and will follow the same dose titration as participants during the double-blind period. All participants, by design, may have the opportunity to receive donanemab at some point in the study; however, assignment in the extension period is double-blind.

Participants whose amyloid plaque reduction as measured by florbetapir F18 PET scans at Visit 8 (Week 24), Visit 15 (Week 52), Visit 21 (Week 76), Visit 28 (Week 102), or Visit 35 (Week 130) meets dose reduction criteria will have a double-blind dose reduction of donanemab to IV placebo for the remaining duration of the study.

FIG. 14 illustrates the study design for clinical protocol. Abbreviations for FIG. 14: IP=investigational product; IV=intravenous; PET=positron emission tomography; Q4W=every 4 weeks; V=Visit. “a” in FIG. 14: Dosing decisions are based on reduction in amyloid burden as determined by the [F18]-florbetapir PET scan at Weeks 24, 52, 76, 102, and 130. “b” in FIG. 14: Donanemab dosing schedule in the extension period up to 78 weeks (maximum duration of treatment period with donanemab is 150 weeks). “c” in FIG. 14: The follow-up period begins 12 weeks after the final administration of investigational product, and Visits 801 through 804 are scheduled as described above. Investigator will be notified if participant is not required to complete all follow-up visits (V801-804). Notes: V601 is optional. For participants who do not complete V601, the procedures will be included in V1. Randomization occurs at V2. FIG. 14 does not differentiate between Cohorts 1 and 2, as both cohorts follow the same study design.

Inclusion Criteria: Male and female participants are eligible to be included in the study only if all of the following criteria apply:
1. 60 to 85 years of age inclusive, at the time of signing the informed consent.
2. Gradual and progressive change in memory function reported by the participant or informant for ≥6 months.
3. An MMSE score of 20 to 28 (inclusive) at screening or visit 1.
4. Meet [F¹⁸]-flortaucipir scan (central read) criteria.
5. Meet [F¹⁸]-florbetapir scan (central read) criteria.
6. Have a study partner who will provide written informed consent to participate, is in frequent contact with the participant (defined as at least 10 hours per week) and will accompany the participant to study visits or be available by telephone at designated times. A second study partner may serve as backup. The study partner(s) is/are required to accompany the participant for signing consent. One study partner is requested to be present or available by phone on all days the C-SSRS/Self-Harm Supplement Form is administered. The study partner must be present on all days the cognitive and functional scales are administered. If a participant has a second study partner, it is preferred that 1 study partner be primarily responsible for the CDR and the Alzheimer's Disease Cooperative Study—Activities of Daily Living Inventory (ADCS-ADL) assessments. Visits requiring the following assessments and scales must have a study partner available by telephone if not accompanying participant at a visit for the following assessments: AEs and concomitant medications; relevant portions of the C-SSRS/Self-Harm Supplement Forms; CDR; and ADCS-ADL. If a study partner must withdraw from study participation, a replacement may be allowed at the investigator's discretion. The replacement will need to sign a separate informed consent on the first visit that he or she accompanies the participant.
7. Have adequate literacy, vision, and hearing for neuropsychological testing in the opinion of the investigator at the time of screening.
8. Are reliable and willing to make themselves available for the duration of the study and are willing to follow study procedures.
9. Stable concomitant symptomatic AD medications and other medication that may impact cognition for at least approximately 30 days prior to randomization (does not apply to topical, as needed [prn], or discontinued medications).
10. Capable of giving signed informed consent which includes compliance with the requirements and restrictions listed in the informed consent form (ICF) and in the protocol.
   Exclusion Criteria: Participants are excluded from the study if any of the following criteria apply: significant neurological disease affecting the central nervous system other than AD, that may affect cognition or ability to complete the study, including but not limited to, other dementias, serious infection of the brain, Parkinson's disease, multiple concussions, or epilepsy or recurrent seizures (except febrile childhood seizures); current serious or unstable illnesses including cardiovascular, hepatic, renal, gastroenterologic, respiratory, endocrinologic, neurologic (other than AD), psychiatric, immunologic, or hematologic disease and other conditions that, in the investigator's opinion, could interfere with the analyses in this study; or has a life expectancy of <24 months; current serious or unstable illnesses including cardiovascular, hepatic, renal, gastroenterologic, respiratory, endocrinologic, neurologic (other than AD), psychiatric, immunologic, or hematologic disease and other conditions that, in the investigator's opinion, could interfere with the analyses in this study; or has a life expectancy of <24 months; participants with any current primary psychiatric diagnosis other than AD if, in the judgment of the investigator, the psychiatric disorder or symptom is likely to confound interpretation of drug effect, affect cognitive assessment, or affect the participant's ability to complete the study; participants with history of schizophrenia or other chronic psychosis are excluded; are, in the judgment of the investigator, actively suicidal and therefore deemed to be at significant risk for suicide; history of alcohol or drug use disorder (except tobacco use disorder) within 2 years before the screening visit; a history of clinically significant multiple or severe drug allergies, significant atopy, or severe posttreatment hypersensitivity reactions (including but not limited to erythema multiforme major, linear immunoglobulin A dermatosis, toxic epidermal necrolysis, and/or exfoliative dermatitis); have any clinically important abnormality at screening, as determined by investigator, in physical or neurological examination, vital signs, ECG, or clinical laboratory test results that could be detrimental to the participant, could compromise the study, or show evidence of other etiologies for dementia; screening MRI which shows evidence of significant abnormality that would suggest another potential etiology for progressive dementia or a clinically significant finding that may impact the participant's ability to safely participate in the study; have any contraindications for MRI, including claustrophobia or the presence of contraindicated metal (ferromagnetic) implants/cardiac pacemaker; have a centrally read MRI demonstrating presence of ARIA-E, >4 cerebral microhemorrhages, more than 1 area of superficial siderosis, any macrohemorrhage or severe white matter disease at screening; have a sensitivity to florbetapir F18 or flortaucipir F18 or contraindication to PET; have poor venous access; present or planned exposure to ionizing radiation that, in combination with the planned administration of study PET ligands, would result in a cumulative exposure that exceeds local recommended exposure limits; alanine aminotransaminase (ALT)≥2.5× the upper limit of normal (ULN) of the performing laboratory, aspartate aminotransferase (AST)≥2.5×ULN, total bilirubin level (TBL)≥1.5×ULN, or alkaline phosphatase (ALP)≥2×ULN at screening; have had prior treatment with a passive anti-amyloid immunotherapy; have received active immunization against Aβ in any other study; are currently enrolled in any other interventional clinical trial involving an investigatory product or any other type of medical research judged not to be scientifically or medically compatible with this study; or have known allergies to donanemab, related compounds, or any components of the formulation. Note: Concurrent use of passive anti-amyloid immunotherapies other than donanemab, such as gantenerumab, lecanemab, or aducanumab, is not permitted during the study.
   Dose Modification: Dose modification of the investigational product (IP) is not permitted in this study, except for participants whose amyloid plaque reduction meets dose reduction criteria or under the conditions described below. The goal of the study is for the participant to be titrated to the target dose. For participants who develop ARIA during the titration period (that is, before the fourth infusion of study drug of the double-blind or of the extension period), the investigator may decide to:
temporarily suspend dosing, then determine if the participant should remain on the pre-suspension dose either temporarily beyond the first 3 doses or throughout the remainder of the treatment period,
continue the same dose either temporarily beyond the first 3 doses or throughout the remainder of the treatment period, or
continue the dosing schedule described above.
   Discontinuation from Study Temporary Discontinuation from Donanemab Treatment due to ARIA-E: Same as described above in Example 2.
   Primary Endpoints: The primary objective of this study is to test the hypothesis that IV infusion of donanemab will slow the cognitive and/or functional decline of AD as measured by iADRS score compared with placebo in the population of participants with low-medium tau pathology at baseline and the overall population. The primary efficacy analysis will utilize the Bayesian Disease Progression Model and be fit on the low-medium tau pathology population at baseline and the overall population for Cohort 2. The primary efficacy analysis may be modified to use an alternative statistical model based on interactions with regulatory agencies and internal decision making. The Bayesian DPM on the iADRS will evaluate possible slowing of disease progression with treatment of donanemab relative to placebo. The primary purpose of the DPM is to estimate a quantity known as the disease progression ratio (DPR), which measures the proportion of disease progression in donanemab-treated participants relative to placebo-treated participants. The key assumption of the DPM model is that it assumes that the treatment effect of donanemab is proportional to placebo over the course of the study. The proportionality assumption is similar to what is made in proportional hazards modeling of time to event data. The model includes diffuse priors on all parameters; therefore, the prior distributions have very little impact on the posterior distributions. No information or knowledge of the effect of donanemab from previous studies will be incorporated into the prior distributions and the inference will be based on Study TRAILBLAZER-ALZ 2 only.

The DPM model is as follows:

$${Y}_{ij} = {γ}_{i} + {e}^{{θ}_{{T}_{i}}} {\sum }_{v = 0}^{j} {α}_{v} + {ε}_{ij} ,$$

where Yᵢⱼ denotes the clinical outcome at visit j for participant i; the clinical outcome score for a participant at baseline (prior to treatment) is Yᵢ₀. The value γᵢ (i=1, 2, . . . , k) represents a subject specific random effect. The parameter Tᵢ denotes the treatment arm for participant i, where Tᵢ has a value of 1 if a participant is randomized to donanemab, and a value of 0 if the participant is randomized to placebo. The parameter αᵥ is the change in mean clinical outcome score for placebo from visit v−1 to v, and εᵢⱼ is the error term. The DPR for donanemab relative to placebo is provided by the parameter e^{θ}. A DPR value less than 1 indicates the donanemab arm is slowing the disease progression relative to placebo, and a DPR value greater than 1 indicates the donanemab arm is worsening the disease progression relative to placebo. Covariates of concomitant AChEI and/or memantine use at baseline (yes/no), age at baseline, and potentially other covariates will be included in the model.

The MMRM analysis will also be assessed for the iADRS. The change from baseline score on the iADRS at each scheduled postbaseline visit during the treatment period will be the dependent variable. The model for the fixed effects will include the following terms: baseline score, pooled investigator, treatment, visit, treatment-by-visit interaction, baseline-by-visit interaction, concomitant AChEI and/or memantine use at baseline (yes/no), and age at baseline. Visit will be considered a categorical variable. The null hypothesis is that the contrast between the donanemab group versus placebo at the last visit equals 0. An unstructured covariance matrix will be used to model the within-subject variance-covariance errors. If the unstructured covariance structure matrix results in a lack of convergence, the following tests will be used in sequence: (1) heterogeneous Toeplitz covariance structure; (2) heterogeneous autoregressive covariance structure; (3) heterogeneous compound symmetry covariance structure; and (4) compound symmetry covariance structure.

The Kenward-Roger approximation will be used to estimate the denominator degrees of freedom. The primary time point for treatment comparison will be at Week 76. The treatment group contrast in least-squares mean progression and its associated p-value and 95% CI will be calculated for the treatment comparison of donanemab versus placebo using the MMRM model specified above. In addition to the DPM and MMRM models described above, the mean for each treatment group over the double-blind period of the study can be modeled using quadratic mixed effects models and natural cubic splines (Chambers and Hastie (ed.), “Statistical Models in S” Chapman & Hall/CRC, 1st edition (1992). The quadratic model has many similar features to the MMRM but makes additional assumptions on the estimates of the longitudinal mean values such that the longitudinal trajectory of each treatment arm is smoothed over the scheduled or observed visit time to allow for a linear or quadratic shape. The Natural Cubic Spline (NCS) model provides a type of smoothing function to the data and can adequately estimate longitudinal trajectories under a variety of shapes (e.g., linear, quadratic, etc.) for each treatment group. The degrees of freedom of the model can be prespecified to establish the level of smoothing of the data. The number and location of the “knots” is utilized to parse out different time periods where the data may transition from one shape to another to provide an adequate fit. The primary time point for treatment comparison will be at Week 76. The variance-covariance structure assumptions of the quadratic or NCS model are the same as the MMRM model and the covariates used in the model would remain unchanged.

The DPM, MMRM, quadratic, and NCS analyses using iADRS will be assessed in the low-medium tau population, overall population, and high tau population.

Secondary Endpoints: Similar to the primary endpoint of iADRS, each of the secondary efficacy outcomes will be assessed using DPM, MMRM, quadratic, and NCS analyses for the low-medium tau population, overall population, and high tau population. These secondary efficacy outcomes include ADAS-Cog₁₃, ADCS-iADL, CDR-SB and MMSE. Longitudinal changes from baseline in amyloid plaque (as measured by [F¹⁸]-florbetapir PET scan) will be analyzed using MMRM including the following terms in the model: baseline biomarker value, treatment, visit, treatment-by-visit interaction, and baseline-by-visit interaction. The change from baseline to endpoint in tau deposition (as measured by flortaucipir PET scan) will be analyzed using an analysis of covariance (ANCOVA) model with terms of baseline value and treatment. Atrophy in vMRI parameters will be analyzed using MMRM including the following terms in the model: treatment, visit, treatment-by-visit interaction, baseline vMRI, intracranial volume, and age at baseline.
Tertiary Exploratory Endpoints: Efficacy analyses will be conducted to evaluate the hypotheses of delayed start disease modification by donanemab on clinical progression as measured by iADRS, CDR-SB, ADCS-iADL, ADAS-Cog₁₃, and MMSE. A statistical method (see Liu-Seifert et al., “A novel approach to delayed-start analyses for demonstrating disease-modifying effects in Alzheimer's Disease.” PLoS ONE, 10(3), (2015)) will be used to analyze each clinical endpoint to compare the treatment efficacy between the early start participants (randomized to donanemab at the beginning of AACI) and delayed start participants (receiving donanemab for the first time in the extension period). The analysis will follow the intent-to-treat (ITT) principle unless otherwise specified. Change in brain amyloid plaque deposition (as measured by [F¹⁸]-florbetapir PET) will be evaluated through Week 154 for the participants who had not met dose reduction criteria during the double-blind period. The brain amyloid plaque deposition (as measured by [F¹⁸]-florbetapir PET) will also be assessed in participants who met dose reduction criteria during double-blind and extension period to evaluate amyloid plaque re-accumulation.
Safety Endpoints: The safety endpoints for this study are:
Standard safety assessments: spontaneously reported adverse events (AEs), clinical laboratory tests, vital sign and body weight measurements, 12-lead electrocardiograms (ECGs), physical and neurological examinations;
MRI (amyloid-related imaging abnormalities [ARIAs] and emergent radiological findings);
Columbia Suicide Severity Rating Scale (C-SSRS): Suicide-related thoughts and behaviors occurring during treatment will be summarized based on responses to the C-SSRS consistent with the C-SSRS Scoring and Data Analysis Guide (Columbia-Suicide Severity Rating Scale (C-SSRS). The Columbia Lighthouse Project website; cssrs.columbia.edu. 2019);
Hepatic safety monitoring: laboratory tests including ALT, AST, ALP, TBL, direct bilirubin, gamma-glutamyl transferase (GGT), and creatine kinase (CK) should be repeated within 48 to 72 hours to confirm any abnormalities. A comprehensive evaluation should be performed to search for possible causes of livery injury if certain significant elevations occur.
   Evaluation of Immunogenicity: Subject samples will be analyzed using a 4-tiered approach. All samples will be assessed in Tier 1 (screening) for the possible presence of ADAs. Samples found to produce a signal above or equal to the screening cut point will be assessed in Tier 2 to confirm specificity to donanemab (confirmation). Any samples confirmed as specific for anti-donanemab antibodies will be reported as “detected.” All samples below the screening cut point (Tier 1) or not confirmed (Tier 2) will be reported as “not detected.” Any “detected” sample in Tier 2 will be assessed in Tier 3 (titer assessment) and Tier 4 (neutralizing antibodies). Anti-drug antibody titer values will be reported from Tier 3 titer assessment. Any samples above the Tier 4 assay cut point will be reported as “detected for neutralizing antibodies”; samples below the assay cut point in Tier 4 will be reported as “not detected for neutralizing antibodies.” The frequency and percentage of subjects with preexisting (baseline) ADAs, ADAs at any time after baseline, and TE ADAs to donanemab will be tabulated. If no ADAs are detected at baseline, TE ADAs are defined as those with a titer 2-fold (1 dilution) greater than the minimal required dilution of the assay. For samples with ADAs detected at baseline, TE ADAs are defined as those with a 4-fold (2 dilutions) increase in titer compared to baseline. For the TE ADA subjects, the distribution of maximum titers will be described. The frequency of neutralizing antibodies may also be tabulated. The relationship between the presence of antibodies to donanemab and PK, PD, safety, and/or efficacy assessment may be assessed.
   Pharmacokinetic Pharmacodynamic Analyses: Compartmental modeling of donanemab PK data using nonlinear mixed effects modeling or other appropriate software may be explored, and population estimates for clearance and central volume of distribution may be reported. Depending on the model selected, other PK parameters may also be reported. Exploratory graphical analyses of the effect of dose level or demographic factors on PK parameters may be conducted. If appropriate, data from other studies of donanemab may be used in this analysis. The PK/PD relationships between plasma donanemab concentration and SUVr, cognitive endpoints, ARIA incidence rate, or other markers of PD activity may be explored graphically. The relationship between the presence of antibodies to donanemab and PK, PD, safety, and/or efficacy may be assessed graphically. To facilitate this modeling, and to ensure that exposure estimates from this study are available at the end of the trial, it is intended that the PK data will be locked after all participants complete Visit 18 (64 weeks of treatment), to allow PK modeling to begin before the end of the trial. No safety or efficacy data will be included in the 64-week PK lock. An Early PK Lock Plan will be developed and implemented prior to this lock, which will specify the safeguards to be taken to ensure the integrity of the study. It is intended that the results of the PK analysis will be provided in a separate report. Additional modeling may be performed based on the results of the graphical analyses.
   Statistical Analysis: The primary efficacy objective of Study AACI is to demonstrate donanemab slows the cognitive and/or functional decline in AD versus placebo as measured by the iADRS through 76 weeks in the population of participants with low-medium tau pathology or the overall population. This study includes high tau participants, a population not studied in AACG (also referred to as TRAILBLAZER-ALZ). The overall population will include all enrolled early symptomatic AD participants with a low-medium or high tau pathology at baseline.

The primary efficacy analysis will be a Bayesian Disease Progression Model (DPM) conducted on the primary outcome iADRS. The primary efficacy analysis will be conducted on two populations: the low-medium tau population at baseline and the overall population. A critical success factor (CSF) will be established of the following form for each population:
Probability (at least X % slowing of disease progression with donanemab relative to placebo for the low-medium tau population)>probability threshold A
Probability (at least X % slowing of disease progression with donanemab relative to placebo for the overall population)>probability threshold B
   The Bayesian DPM will be utilized to calculate the posterior probabilities of at least X % slowing for each population. If the posterior probability for the low-medium tau population exceeds the pre-specified probability threshold A, or the posterior probability for the overall population exceeds the pre-specified probability threshold B, the trial will have been considered to have met its primary endpoint. If one of the CSFs is met and the other is not, the CSF that was not met may be re-tested at an alternative pre-specified probability threshold in a similar fashion to recycle alpha in a frequentist framework (Millen et. al., “Chain Procedures: a class of flexible closed testing procedures with clinical trial applications.” Stat Biopharm. Res. 2011 3(1):14-30). The exact CSF for each population, and the potential CSFs if one CSF is met and not the other, will be pre-specified in the Study SAP prior to unblinding Cohort 2 of the trial. The CSFs will be determined via simulation and will ensure that the false positive rate (probability of meeting at least one of the CSFs under the null) is below 2.5% under a variety of null scenarios (e.g., different rates of placebo decline, variability assumptions, etc.).

Major secondary hypotheses defined in a similar manner are that donanemab is superior to placebo with regard to:
Clinical progression in participants with early symptomatic AD through Week 76, as measured by MMSE, ADAS-Cog13, CDR-SB, and ADCS-iADL;
Brain amyloid deposition at Week 76 measured by florbetapir F18 PET scan;
Brain tau deposition at Week 76 measured by flortaucipir F18 PET scan; and
Brain region volumes at Week 76 measured by vMRI.
   Final Summary: Study AACI expands the participant population compared to the prior Phase 2 study by including participants with high tau pathology. The primary analysis will test low-medium tau pathology population and the overall population and adds a long-term extension period designed to further evaluate donanemab efficacy and safety over time. The results of Study AACI may result in a significant slowing of disease progression, as measured by the integrated Alzheimer's Disease Rating Scale as well as deep and rapid reduction in amyloid plaque level with a concomitant decrease in tau spreading.

### EXEMPLIFIED EMBODIMENTS OF THE PRESENT DISCLOSURE

The following provides embodiments set forth throughout this disclosure.

1. A method of treating or preventing a disease characterized by amyloid beta plaques in the brain of a human subject comprising:
i) administering to the human subject one or more first doses of about 100 mg to about 700 mg of an anti-N3pG Aβ antibody, wherein each first dose is administered once about every four weeks; and
ii) about four weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

2. The embodiment of 1, wherein the human subject is administered the first dose once, two times, or three times before administering the second dose.

3. The embodiment of 1 or 2, wherein the human subject is administered first doses of about 700 mg.

4. The embodiment of any one of 1 to 3, wherein the human subject is administered one or more second doses of about 800 mg, about 900 mg, about 1000 mg, about 1100 mg, about 1200 mg, about 1300 mg, or about 1400 mg.

5. The embodiment of any one of 1 to 4, wherein the human subject is administered one or more second doses of about 1400 mg.

6. The embodiment of any one of 1 to 5, wherein the anti-N3pGlu Aβ antibody is administered to the human subject for a duration of up to 72 weeks, until normal level of amyloid is achieved, or until reduction/removal of Aβ from the brain of the subject stops.

7. The embodiment of any one of 1 to 6, wherein the anti-N3pGlu Aβ antibody is administered to the human subject until the amyloid plaque level in the patient is about 25 centiloids or lower.

8. The embodiment of any one of 1 to 6, wherein the anti-N3pGlu Aβ antibody is administered to the human subject until the amyloid plaque level in the human subject is about 25 centiloids or lower for two consecutive PET imaging scans, optionally, wherein the two consecutive PET imaging scans are at least 6 months apart, or about 11 centiloids or lower for one PET imaging scan.

9. The embodiment of any one of 1 to 6, wherein the human subject is administered three first doses of 700 mg once every four weeks and then second doses of 1400 mg once every four weeks for a duration of up to 72 weeks.

10. The embodiment of any one of 1 to 6, wherein the human subject is administered three first doses of 700 mg once every four weeks and then second doses of 1400 mg once every four weeks until the amyloid plaque level in the subject is about 25 centiloids or lower.

11. The embodiment of any one of 1 to 6, wherein the human subject is administered three first doses of 700 mg once every four weeks and then second doses of 1400 mg once every four weeks until amyloid plaque level in the subject is about 25 centiloids or lower for two consecutive PET imaging scans, optionally, wherein the two consecutive PET imaging scans are at least 6 months apart, or about 11 centiloids or lower for one PET imaging scan.

12. The embodiment of any one of 1 to 11, wherein the human subject is administered the second dose for a duration sufficient to treat or prevent the disease.

13. The embodiment of any one of 1 to 12, wherein the treatment or prevention of the disease causes i) reduction in Aβ plaques in the brain of the human subject and/or ii) slows cognitive or functional decline in the human subject.

14. The embodiment of 13, wherein the reduction in Aβ plaques in the brain of the human subject is determined by amyloid PET brain imaging or a diagnostic that detects a biomarker for Aβ.

15. The embodiment of 13 or 14, wherein the second dose is administered to the human subject until there is about 20-100% reduction in Aβ plaques in the brain of the human subject.

16. The embodiment of 15, wherein the Aβ plaques in the brain of the human subject are reduced by about 20%, about 25%, about 30%, about 35%, about 40%, about 45%, about 50%, about 75% or about 100%.

17. The embodiment of any one of 1 to 14, wherein the second dose is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

18. The embodiment of any one of 1 to 17, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

19. The embodiment of any one of 1 to 18, wherein the human subject is an early symptomatic AD patient.

20. The embodiment of 19, wherein the human subject has prodromal AD and mild dementia due to AD.

21. The embodiment of anyone of 1-20, wherein the human subject has: i) very low to moderate tau burden or has been determined to have very low to moderate tau burden, ii) low to moderate tau burden or has been determined to have low to moderate tau burden, iii) very low to moderate tau burden or has been determined to have very low to moderate tau burden and one or two alleles of APOE4, iv) low to moderate tau burden or has been determined to have low to moderate tau burden and one or two alleles of APOE4, or v) one or two alleles of APOE4.

22. The embodiment of 21, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

23. The embodiment of anyone of 1-20, wherein the human subject i) does not have high tau burden or has been determined to not have a high tau burden or ii) carries one or two alleles of APOE4 and does not have high tau burden or has been determined to not have a high tau burden.

24. The embodiment of 23, wherein the human subject has high tau burden if the tau burden as measured by PET brain imaging is above 1.46 SUVr.

25. The embodiment of 21 or 23, wherein the tau burden of the human subject is determined using PET brain imaging or a diagnostic that detects a biomarker for tau.

26. The embodiment of any one of 1-25, wherein the anti-N3pGlu Aβ antibody comprises a light chain (LC) and a heavy chain (HC), wherein the LC comprises the amino acid sequence of SEQ ID NO: 3 and the HC comprises the amino acid sequence of SEQ ID NO: 4.

27. The embodiment of any one of 1-26, wherein the anti-N3pGlu Aβ antibody comprises two light chains and two heavy chains, wherein the LC comprises the amino acid sequence of SEQ ID NO: 3 and the HC comprises the amino acid sequence of SEQ ID NO: 4.

28. An anti-N3pGlu Aβ antibody for use in the treatment or prevention of a disease characterized by Aβ plaques in the brain of a human subject,
wherein the anti-N3pGlu Aβ antibody is for administration of one or more first doses of about 100 mg to about 700 mg, wherein each first dose is administered once about every 4 weeks followed by administration of one or more second doses of greater than 700 mg to about 1400 mg four weeks after administering the one or more first doses, wherein each second dose is administered once about every 4 weeks, and
wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

29. An anti-N3pGlu Aβ antibody for use in the treatment or prevention of a disease characterized by Aβ plaques in the brain of a human subject,
wherein one or more first doses of about 100 mg to about 700 mg of the antibody are administered wherein each first dose is administered once about every 4 weeks followed by administration of one or more second doses of greater than 700 mg to about 1400 mg four weeks after administering the one or more first doses wherein each second dose is administered once about every 4 weeks, and
wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

30. The embodiment of 28 or 29, wherein the human subject is administered the first dose once, two times, or three times before administering the second doses.

31. The embodiment of any one of 28-30, wherein the human subject is administered first doses of about 700 mg.

32. The embodiment of any one of 28-31, wherein the human subject is administered one or more second doses of about 800 mg, about 900 mg, about 1000 mg, about 1100 mg, about 1200 mg, about 1300 mg, or about 1400 mg.

33. The embodiment of any one of 28-32, wherein the human subject is administered one or more second doses of about 1400 mg.

34. The embodiment of any one of 28-33, wherein the anti-N3pGlu Aβ antibody is administered to the human subject for a duration of up to 72 weeks or until a normal level of amyloid is achieved.

35. The embodiment of any one of 28-34, wherein the anti-N3pGlu Aβ antibody is administered to the human subject until the amyloid plaque level in the patient is about 25 centiloids or lower.

36. The embodiment of any one of 28-34, wherein the anti-N3pGlu Aβ antibody is administered to the human subject until the amyloid plaque level in the human subject is about 25 centiloids or lower for two consecutive PET imaging scans, optionally, wherein the two consecutive PET imaging scans are at least 6 months apart, or about 11 centiloids or lower for one PET imaging scan.

37. The embodiment of any one of 28-34, wherein the human subject is administered three first doses of 700 mg once every four weeks and then second doses of 1400 mg once every four weeks for a duration of up to 72 weeks.

38. The embodiment of any one of 28-34, wherein the human subject is administered three first doses of 700 mg once every four weeks and then second doses of 1400 mg once every four weeks until the amyloid plaque level in the patient is about 25 centiloids or lower.

39. The embodiment of any one of 28-34, wherein the human subject is administered three first doses of 700 mg once every four weeks and then second doses of 1400 mg once every four weeks until amyloid plaque level in the subject is about 25 centiloids or lower for two consecutive PET imaging scans, optionally, wherein the two consecutive PET imaging scans are at least 6 months apart, or about 11 centiloids or lower for one PET imaging scan.

40. The embodiment of any one of 28-39, wherein the human subject is administered the second dose for a duration sufficient to treat or prevent the disease.

41. The embodiment of any one of 28-40, wherein the treatment or prevention of the disease causes i) reduction in Aβ plaques in the brain of the human subject and/or ii) slows cognitive or functional decline in the human subject.

42. The embodiment of 41, wherein the reduction in Aβ plaques in the brain of the human subject is determined by amyloid PET brain imaging or a diagnostic that detects a biomarker for Aβ.

43. The embodiment of 41 or 42, wherein the second dose is administered to the human subject until there is about 20-100% reduction in Aβ plaques in the brain of the human subject.

44. The embodiment of 43, wherein the Aβ plaques in the brain of the human subject are reduced by about 20%, about 25%, about 30%, about 35%, about 40%, about 45%, about 50%, about 75% or about 100%.

45. The embodiment of 43 or 44, wherein the Aβ plaques in the brain of the patient are reduced by about 100%.

46. The embodiment of any one of 28 to 42, wherein the second dose is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

47. The embodiment of any one of 28 to 46, wherein the disease characterized by Aβ deposit in the brain of the human subject is selected from preclinical Alzheimer's disease, clinical AD, prodromal AD, mild AD, moderate AD, severe AD, Down's syndrome, clinical cerebral amyloid angiopathy, or pre-clinical cerebral amyloid angiopathy.

48. The embodiment of any one of 28-47, wherein the human subject is an early symptomatic AD patient or wherein the human subject has prodromal AD and mild dementia due to AD.

49. The embodiment of any one of 28-48, wherein the human subject has: i) very low to moderate tau burden or has been determined to have very low to moderate tau burden, ii) low to moderate tau burden or has been determined to have low to moderate tau burden, iii) very low to moderate tau burden or has been determined to have very low to moderate tau burden and one or two alleles of APOE4, iv) low to moderate tau burden or has been determined to have low to moderate tau burden and one or two alleles of APOE4, or v) one or two alleles of APOE4.

50. The embodiment of 49, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

51. The embodiment of any one of 28 to 48, wherein the human subject i) does not have high tau burden or has been determined to not have a high tau burden or ii) carries one or two alleles of APOE4 and does not have high tau burden or has been determined to not have a high tau burden.

52. The embodiment of 51, wherein the human subject has high tau burden if the tau burden as measured by PET brain imaging is above 1.46 SUVr.

53. The embodiment of 49 or 51, wherein the tau burden of the human subject is determined using PET brain imaging or a diagnostic that detects a biomarker for tau.

54. The embodiment of any one of 28 to 53, wherein the anti-N3pGlu Aβ antibody comprises a LC and a HC, wherein the LC comprises the amino acid sequence of SEQ ID NO: 3 and the HC comprises the amino acid sequence of SEQ ID NO: 4.

55. The embodiment of any one of 28 to 54, wherein the anti-N3pGlu Aβ antibody comprises two light chains and two heavy chains, wherein the LC comprises the amino acid sequence of SEQ ID NO: 3 and the HC comprises the amino acid sequence of SEQ ID NO: 4.

56. Use of an anti-N3pGlu Aβ antibody in the manufacture of a medicament for treatment or prevention of a disease characterized by Aβ plaques in the brain of a human subject,
wherein one or more first doses of about 100 mg to about 700 mg of the antibody are administered, wherein each first dose is administered once about every 4 weeks followed by administration of one or more second doses of greater than 700 mg to about 1400 mg four weeks after administering the one or more first doses, wherein each second dose is administered once about every 4 weeks, and
wherein the anti-N3pGlu Aβ antibody comprises a LCVR and a HCVR, wherein the LCVR comprises the amino acid sequence of SEQ ID NO: 1 and the HCVR comprises the amino acid sequence of SEQ ID NO: 2.

57. The embodiment of 56, wherein the human subject is administered the first dose once, two times, or three times before administering the second doses.

58. The embodiment of 56 or 57, wherein the human subject is administered three first doses of about 700 mg.

59. The embodiment of anyone of 56-58, wherein the human subject is administered one or more second doses of about 800 mg, about 900 mg, about 1000 mg, about 1100 mg, about 1200 mg, about 1300 mg, or about 1400 mg.

60. The embodiment of any one of 56-59, wherein the human subject is administered one or more second doses of about 1400 mg.

61. The embodiment of any one of 56-60, wherein the anti-N3pGlu Aβ antibody is administered to the human subject for a duration of up to 72 weeks or until normal level of amyloid is achieved.

62. The embodiment of any one of 56-61, wherein the anti-N3pGlu Aβ antibody is administered to the human subject until the amyloid plaque level in the patient is about 25 centiloids or lower.

63. The embodiment of any one of 56-61, wherein the anti-N3pGlu Aβ antibody is administered to the human subject until the amyloid plaque level in the patient is about 25 centiloids or lower for two consecutive PET imaging scans, optionally, wherein the two consecutive PET imaging scans are at least 6 months apart, or about 11 centiloids or lower for one PET imaging scan. 64. The embodiment of any one of 56-61, wherein the human subject is administered three first doses of 700 mg once every four weeks and then second doses of 1400 mg once every four weeks for a duration of up to 72 weeks.

65. The embodiment of any one of 56-61, wherein the human subject is administered three first doses of 700 mg once every four weeks and then second doses of 1400 mg once every four weeks until the amyloid plaque level in the patient is about 25 centiloids or lower.

66. The embodiment of any one of 56-61, wherein the human subject is administered three first doses of 700 mg once every four weeks and then second doses of 1400 mg once every four weeks until amyloid plaque level in the patient is about 25 centiloids or lower for two consecutive PET imaging scans, optionally, wherein the two consecutive PET imaging scans are at least 6 months apart, or about 11 centiloids or lower for one PET imaging scan.

67. The embodiment of any one of 56-66, wherein the human subject is administered the second dose for a duration sufficient to treat or prevent the disease.

68. The embodiment of any one of 56-67, wherein the treatment or prevention of the disease causes i) reduction in Aβ plaques in the brain of the human subject and/or ii) slows cognitive or functional decline in the human subject.

69. The embodiment of 68, wherein the reduction in Aβ plaques in the brain of the human subject is determined by amyloid PET brain imaging or a diagnostic that detects a biomarker for Aβ.

70. The embodiment of 68 or 69, wherein the second dose is administered to the human subject until there is about 20-100% reduction in Aβ plaques in the brain of the human subject.

71. The embodiment of 70, wherein the Aβ plaques in the brain of the human subject are reduced by about 20%, about 25%, about 30%, about 35%, about 40%, about 45%, about 50%, about 75% or about 100%.

72. The embodiment of 70 or 71, wherein the Aβ plaques in the brain of the patient are reduced by 100%.

73. The embodiment of any one of 56 to 72, wherein the second dose is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

74. The embodiment of any one of 56 to 73, wherein the disease characterized by Aβ deposit in the brain of the human subject is selected from preclinical Alzheimer's disease, clinical AD, prodromal AD, mild AD, moderate AD, severe AD, Down's syndrome, clinical cerebral amyloid angiopathy, or pre-clinical cerebral amyloid angiopathy.

75. The embodiment of any one of 56 to 74, wherein the human subject is an early symptomatic AD patient or wherein the human subject has prodromal AD or mild dementia due to AD.

76. The embodiment of any one of 56 to 75, wherein the human subject has: i) very low to moderate tau burden or has been determined to have very low to moderate tau burden, ii) low to moderate tau burden or has been determined to have low to moderate tau burden, iii) very low to moderate tau burden or has been determined to have very low to moderate tau burden and one or two alleles of APOE4, iv) low to moderate tau burden or has been determined to have low to moderate tau burden and one or two alleles of APOE4, or v) one or two alleles of APOE4.

77. The embodiment of 76, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

78. The embodiment of any one of 56-75, wherein the human subject i) does not have high tau burden or has been determined to not have a high tau burden or ii) carries one or two alleles of APOE4 and does not have high tau burden or has been determined to not have a high tau burden.

79. The embodiment of 78, wherein the human subject has high tau burden if the tau burden as measured by PET brain imaging is above 1.46 SUVr.

80. The embodiment of 76 or 78, wherein the tau burden of the human subject is determined using tau PET brain imaging or a diagnostic that detects a biomarker for tau.

81. The embodiment of any one of 56 to 80, wherein the anti-N3pGlu Aβ antibody comprises a LC and a HC, wherein the LC comprises the amino acid sequence of SEQ ID NO: 3 and the HC comprises the amino acid sequence of SEQ ID NO: 4.

82. The embodiment of any one of 56 to 81, wherein the anti-N3pGlu Aβ antibody comprises two light chains and two heavy chains, wherein the LC comprises the amino acid sequence of SEQ ID NO: 3 and the HC comprises the amino acid sequence of SEQ ID NO: 4.

83. A method of treating or preventing a disease characterized by amyloid beta plaques in the brain of a human subject who has been determined to have i) very low to moderate tau burden or low to moderate tau burden or ii) very low to moderate tau burden or low to moderate tau burden and one or two alleles of APOE4 comprising:
i) administering to the human subject one or more first doses of about 100 mg to about 700 mg of an anti-N3pG Aβ antibody, wherein each first dose is administered once about every 4 weeks; and
ii) 4 weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

84. A method of treating or preventing a disease characterized by amyloid beta plaques in the brain of a human subject comprising:
determining whether the human subject has very low to moderate tau burden or low to moderate tau burden and/or one or two alleles of APOE4; and if the human subject has very low to moderate tau burden or low to moderate tau burden and/or one or two alleles of APOE4, then:
i) administering to the human subject one or more first doses of about 100 mg to about 700 mg of an anti-N3pG Aβ antibody, wherein each first dose is administered once about every 4 weeks; and
ii) 4 weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

85. A method of treating or preventing a disease characterized by amyloid beta plaques in the brain of a human subject who has been determined i) as not having high tau burden or ii) having one or two alleles of APOE4 and not having high tau burden comprising:
i) administering to the human subject one or more first doses of about 100 mg to about 700 mg of an anti-N3pG Aβ antibody, wherein each first dose is administered once about every 4 weeks; and
ii) 4 weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

86. A method of treating or preventing a disease characterized by amyloid beta plaques in the brain of a human subject comprising:
determining whether the human subject has i) high tau burden or ii) high tau burden and one or two alleles of APOE4; and if the human subject does not have high tau burden or if the human subject does not have high tau burden and has one or two alleles of APOE4, then:
i) administering to the human subject one or more first doses of about 100 mg to about 700 mg of an anti-N3pG Aβ antibody, wherein each first dose is administered once about every 4 weeks; and
ii) 4 weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

87. The embodiment of any one of 83-86, wherein the human subject is administered the first dose once, two times, or three times before administering the second dose.

88. The embodiment of any one of 83-87, wherein the human subject is administered first doses of about 700 mg.

89. The embodiment of any one of 83-88, wherein the human subject is administered one or more second doses of about 800 mg, about 900 mg, about 1000 mg, about 1100 mg, about 1200 mg, about 1300 mg, or about 1400 mg.

90. The embodiment of any one of 83-89, wherein the human subject is administered one or more second doses of about 1400 mg.

91. The embodiment of any one of 83-90, wherein the anti-N3pGlu Aβ antibody is administered to the human subject for a duration of up to 72 weeks or until normal level of amyloid is achieved.

92. The embodiment of any one of 83-91, wherein the anti-N3pGlu Aβ antibody is administered to the human subject until the amyloid plaque level in the human subject is about 25 centiloids or lower.

93. The embodiment of any one of 83-91, wherein the anti-N3pGlu Aβ antibody is administered to the human subject until the amyloid plaque level in the human subject is about 25 centiloids or lower for two consecutive PET imaging scans, optionally, wherein the two consecutive PET imaging scans are 6 months apart, or about 11 centiloids or lower for one PET imaging scan.

94. The embodiment of any one of 83-91, wherein the human subject is administered three first doses of 700 mg once every four weeks and then second doses of 1400 mg once every four weeks for a duration of up to 72 weeks.

95. The embodiment of any one of 83-91, wherein the human subject is administered three first doses of 700 mg once every four weeks and then second doses of 1400 mg once every four weeks until the amyloid plaque level in the subject is about 25 centiloids or lower.

96. The embodiment of any one of 83-91, wherein the human subject is administered three first doses of 700 mg once every four weeks and then second doses of 1400 mg once every four weeks until amyloid plaque level in the patient is about 25 centiloids or lower for two consecutive PET imaging scans, optionally, wherein the two consecutive PET imaging scans are at least 6 months apart, or about 11 centiloids or lower for one PET imaging scan.

97. The embodiment of any one of 83-96, wherein the human subject is administered the second dose for a duration sufficient to treat or prevent the disease.

98. The embodiment of any one of 83-97, wherein the treatment or prevention of the disease causes i) reduction in Aβ plaques in the brain of the human subject and/or ii) slows cognitive or functional decline in the human subject.

99. The embodiment of 98, wherein the reduction in Aβ plaques in the brain of the human subject is determined by amyloid PET brain imaging or a diagnostic that detects a biomarker for Aβ.

100. The embodiment of 98 or 99, wherein the second dose is administered to the human subject until there is about 20-100% reduction in Aβ plaques in the brain of the human subject.

101. The embodiment of 100, wherein the Aβ plaques in the brain of the human subject are reduced by about 20%, about 25%, about 30%, about 35%, about 40%, about 45%, about 50%, about 75% or about 100%.

102. The embodiment of any one of 83 to 99, wherein the second dose is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

103. The embodiment of any one of 83-102, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

104. The embodiment of any one of 83-103, wherein the human subject is an early symptomatic AD patient.

105. The embodiment of 104, wherein the human subject has prodromal AD and mild dementia due to AD.

106. The embodiment of claim 83 or 84, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

107. The embodiment of 85 or 86, wherein the human subject has high tau burden if the tau burden as measured by PET brain imaging is above 1.46 SUVr.

108. The embodiment of any one of 83-86, wherein the tau burden of the human subject is determined using PET brain imaging or a diagnostic that detects a biomarker for tau.

109. The embodiment of any one of 83-108, wherein the anti-N3pGlu Aβ antibody comprises a light chain (LC) and a heavy chain (HC), wherein the LC comprises the amino acid sequence of SEQ ID NO: 3 and the HC comprises the amino acid sequence of SEQ ID NO: 4.

110. The embodiment of any one of 83-109, wherein the anti-N3pGlu Aβ antibody comprises two light chains and two heavy chains, wherein the LC comprises the amino acid sequence of SEQ ID NO: 3 and the HC comprises the amino acid sequence of SEQ ID NO: 4.

111. A method of treating or preventing a disease characterized by amyloid beta plaques in the brain of a human subject comprising:
administering to the human subject an effective amount of an anti-N3pGlu Aβ antibody, wherein the human subject has been determined i) as having a low to moderate tau burden or a very low to moderate tau burden, ii) as having a low to moderate tau burden or a very low to moderate tau burden and one or two alleles of APOE4, or iii) as having one or two alleles of APOE4.

112. A method of treating or preventing a disease characterized by amyloid beta plaques in the brain of a human subject comprising:
determining whether the human subject has low to moderate tau burden or a very low to moderate tau burden; and if the human subject has low to moderate tau burden or a very low to moderate tau burden, then: administering to the human subject an effective amount of an anti-N3pGlu Aβ antibody or
determining whether the human subject has i) low to moderate tau burden and one or two alleles of APOE4 or ii) a very low to moderate tau burden and one or two alleles of APOE4; and if the human subject has i) low to moderate tau burden and one or two alleles of APOE4 or ii) a very low to moderate tau burden and one or two alleles of APOE4, then: administering to the human subject an effective amount of an anti-N3pGlu Aβ antibody.

113. A method of treating or preventing a disease characterized by amyloid beta plaques in the brain of a human subject comprising:
administering to the human subject an effective amount of an anti-N3pGlu Aβ antibody, wherein the human subject has been determined as i) not having a high tau burden or ii) not having a high tau burden and one or two alleles of APOE4.

114. A method of treating or preventing a disease characterized by amyloid beta plaques in the brain of a human subject comprising:
determining whether the human subject has i) high tau burden or ii) high tau burden and one or two alleles of APOE4; and if the human subject i) does not have high tau burden or ii) has one or two alleles of APOE4 and does not have a high tau burden, then:
administering to the human subject an effective amount of an anti-N3pGlu Aβ antibody.

115. The embodiment of any one of 111-114, wherein the human subject is administered the effective amount of the anti-N3pGlu Aβ antibody for a duration sufficient to treat or prevent the disease.

116. The embodiment of any one of 111-115, wherein the treatment or prevention of the disease causes i) reduction in Aβ plaques in the brain of the human subject and/or ii) slows cognitive or functional decline in the human subject.

117. The embodiment of 116, wherein the reduction in Aβ plaques in the brain of the human subject is determined by amyloid PET brain imaging or a diagnostic that detects a biomarker for Aβ.

118. The embodiment of 116 or 117, wherein the effective dose of the anti-N3pGlu Aβ antibody is administered to the human subject until there is about 20-100% reduction in Aβ plaques in the brain of the human subject.

119. The embodiment of 118, wherein the Aβ plaques in the brain of the human subject are reduced by about 20%, about 25%, about 30%, about 35%, about 40%, about 45%, about 50%, about 75% or about 100%.

120. The embodiment of any one of 111 to 119, wherein the effective dose of the anti-N3pGlu Aβ antibody is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

121. The embodiment of any one of 111 to 120, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

122. The embodiment of any one of 111 to 121, wherein the human subject is an early symptomatic AD patient.

123. The embodiment of 122, wherein the human subject has prodromal AD and mild dementia due to AD.

124. The embodiment of 111 or 112, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

125. The embodiment of 113 or 114, wherein the human subject has high tau burden if the tau burden as measured by PET brain imaging is above 1.46 SUVr.

126. The embodiment of any one of 111-114, wherein the tau burden of the human subject is determined using PET brain imaging or a diagnostic that detects a biomarker for tau.

127. A method of decreasing/preventing further increase of tau burden, or slowing the rate of tau accumulation in the temporal lobe, the occipital lobe, the parietal lobe, or the frontal lobe of a human brain comprising:
i) administering to the human subject one or more first doses of about 100 mg to about 700 mg of an anti-N3pG Aβ antibody, wherein each first dose is administered once about every four weeks; and
ii) about four weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

128. A method of treating or preventing a disease characterized by amyloid beta plaques in the brain of a human subject who has been determined to i) have tau burden in the temporal lobe, the occipital lobe, the parietal lobe, or the frontal lobe of the brain wherein the method comprises
i) administering to the human subject one or more first doses of about 100 mg to about 700 mg of an anti-N3pG Aβ antibody, wherein each first dose is administered once about every four weeks; and
ii) about four weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

129. A method of treating or preventing a disease characterized by amyloid beta plaques in the brain of a human subject comprising:
determining whether the human subject has tau burden in the temporal lobe, the occipital lobe, the parietal lobe, or the frontal lobe of the brain and if the human subject has tau burden in the temporal lobe, the occipital lobe, the parietal lobe, or the frontal lobe of the brain, then:
i) administering to the human subject one or more first doses of about 100 mg to about 700 mg of an anti-N3pG Aβ antibody, wherein each first dose is administered once about every four weeks; and
ii) about four weeks after administering the one or more first doses, administering to the human subject one or more second doses of greater than 700 mg to about 1400 mg of the anti-N3pG Aβ antibody, wherein each second dose is administered once about every 4 weeks,
wherein the anti-N3pGlu Aβ antibody comprises a light chain variable region (LCVR) and a heavy chain variable region (HCVR), wherein the LCVR comprises the amino acid sequence of SEQ ID NO: 1 and the HCVR comprises the amino acid sequence of SEQ ID NO: 2.

130. The embodiment of any one of 127-129, wherein the human subject has tau burden in the posterolateral temporal lobe or the temporal lobe of the brain.

131. The embodiment of any one of 127-130, wherein the human subject has tau burden in the occipital lobe of the brain.

132. The embodiment of any one of 127-131, wherein the human subject has tau burden in the parietal lobe of the brain.

133. The embodiment of any one of 127-132, wherein the human subject has tau burden in the frontal lobe of the brain.

134. The embodiment of any one of 127-133, wherein the human subject has tau burden in the posterolateral temporal (PLT) and/or occipital lobe of the brain.

135. The embodiment of any one of 127-134, wherein the human subject has tau burden in i) parietal or precuneus region or ii) in frontal region along with tau burden in PLT or occipital regions of the brain.

136. The embodiment of any one of 127-135, wherein the human subject has tau burden i) isolated to frontal lobe or ii) in regions of the temporal lobe that do not include the posterolateral temporal region (PLT) of the brain.

137. The embodiment of any one of 127-136, wherein the human subject has tau burden in posterior-lateral temporal lobe, occipital lobe, and parietal lobe of the brain.

138. The embodiment of any one of 127-137, wherein the human subject has tau burden in posterior-lateral temporal lobe, occipital lobe, parietal lobe, and frontal lobe of the brain.

139. The embodiment of any one of 127-138, wherein the human subject has tau burden in posterior-lateral temporal lobe, occipital lobe, parietal lobe and/or frontal lobe of the brain.

140. The embodiment of any one of 127-139, wherein the human subject is administered the first dose once, two times, or three times before administering the second dose.

141. The embodiment of any one of 127-140, wherein the human subject is administered first doses of about 700 mg.

142. The embodiment of any one of 127 to 141, wherein the human subject is administered one or more second doses of about 800 mg, about 900 mg, about 1000 mg, about 1100 mg, about 1200 mg, about 1300 mg, or about 1400 mg.

143. The embodiment of any one of 127 to 142, wherein the human subject is administered one or more second doses of about 1400 mg.

144. The embodiment of any one of 127 to 143, wherein the anti-N3pGlu Aβ antibody is administered to the human subject for a duration of up to 72 weeks or until normal level of amyloid is achieved.

145. The embodiment of any one of 127 to 144, wherein the anti-N3pGlu Aβ antibody is administered to the human subject until the amyloid plaque level in the patient is about 25 centiloids or lower.

146. The embodiment of any one of 127 to 145, wherein the anti-N3pGlu Aβ antibody is administered to the human subject until the amyloid plaque level in the human subject is about 25 centiloids or lower for two consecutive PET imaging scans, optionally, wherein the two consecutive PET imaging scans are at least 6 months apart, or about 11 centiloids or lower for one PET imaging scan.

147. The embodiment of any one of 127 to 146, wherein the human subject is administered three first doses of 700 mg once every four weeks and then second doses of 1400 mg once every four weeks for a duration of up to 72 weeks.

148. The embodiment of any one of 127 to 147, wherein the human subject is administered three first doses of 700 mg once every four weeks and then second doses of 1400 mg once every four weeks until the amyloid plaque level in the subject is about 25 centiloids or lower.

149. The embodiment of any one of 127 to 148, wherein the human subject is administered three first doses of 700 mg once every four weeks and then second doses of 1400 mg once every four weeks until amyloid plaque level in the subject is about 25 centiloids or lower for two consecutive PET imaging scans, optionally, wherein the two consecutive PET imaging scans are at least 6 months apart, or about 11 centiloids or lower for one PET imaging scan.

150. The embodiment of any one of 127 to 149, wherein the human subject is administered the second dose for a duration sufficient to treat or prevent the disease.

151. The embodiment of any one of 127 to 150, wherein the treatment or prevention of the disease causes i) reduction in Aβ plaques in the brain of the human subject and/or ii) slows cognitive or functional decline in the human subject.

152. The method of claim 151, wherein the reduction in Aβ plaques in the brain of the human subject is determined by amyloid PET brain imaging or a diagnostic that detects a biomarker for Aβ.

153. The embodiment of 151 or 152, wherein the second dose is administered to the human subject until there is about 20-100% reduction in Aβ plaques in the brain of the human subject.

154. The embodiment of 153, wherein the Aβ plaques in the brain of the human subject are reduced by about 20%, about 25%, about 30%, about 35%, about 40%, about 45%, about 50%, about 75% or about 100%.

155. The embodiment of any one of 127 to 154, wherein the second dose is administered to the human subject until the Aβ plaques in the brain of the human subject are reduced by i) about an average of about 25 centiloids to about 100 centiloids, ii) about an average of about 50 centiloids to about 100 centiloids, iii) about 100 centiloids, or iv) about 84 centiloids.

156. The embodiment of any one of 127 to 155, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

157. The embodiment of any one of 127 to 156, wherein the human subject is an early symptomatic AD patient.

158. The embodiment of 157, wherein the human subject has prodromal AD and mild dementia due to AD.

159. The embodiment of 127-158, wherein the human subject has: i) very low to moderate tau burden or has been determined to have very low to moderate tau burden, or ii) low to moderate tau burden or has been determined to have low to moderate tau burden.

160. The embodiment of 159, wherein the human subject has i) very low to moderate tau burden if the tau burden as measured by PET brain imaging is ≤1.46 SUVr or ii) low to moderate tau burden if the tau burden as measured by PET brain imaging is from 1.10 SUVr to 1.46 SUVr.

161. The embodiment of any one of 127 to 160, wherein the human subject does not have high tau burden or has been determined to not have a high tau burden.

162. The embodiment of 161, wherein the human subject has high tau burden if the tau burden as measured by PET brain imaging is above 1.46 SUVr.

163. The embodiment of 159 or 161, wherein the tau burden of the human subject is determined using PET brain imaging or a diagnostic that detects a biomarker for tau.

164. The embodiment of any one of 127 to 163, wherein the anti-N3pGlu Aβ antibody comprises a light chain (LC) and a heavy chain (HC), wherein the LC comprises the amino acid sequence of SEQ ID NO: 3 and the HC comprises the amino acid sequence of SEQ ID NO: 4.

165. The embodiment of any one of 127 to 164, wherein the anti-N3pGlu Aβ antibody comprises two light chains and two heavy chains, wherein the LC comprises the amino acid sequence of SEQ ID NO: 3 and the HC comprises the amino acid sequence of SEQ ID NO: 4.

166. The embodiment of any one of 127-165, wherein the patient has one or two alleles of APOE4.

167. A method of decreasing/preventing further increase of tau burden or slowing the rate of tau accumulation in the temporal lobe, the occipital lobe, the parietal lobe, or the frontal lobe of a human brain comprising administering an anti-N3pGlu Aβ antibody to the human subject.

168. A method of treating or preventing a disease characterized by amyloid beta plaques in the brain of a human subject who has been determined to have tau burden in the temporal lobe, the occipital lobe, the parietal lobe, or the frontal lobe of the brain wherein the method comprises administering an anti-N3pGlu Aβ antibody to the human subject.

169. A method of treating or preventing a disease characterized by amyloid beta plaques in the brain of a human subject comprising: determining whether the human subject has tau burden in the temporal lobe, the occipital lobe, the parietal lobe, or the frontal lobe of the brain and if the human subject has tau burden in the temporal lobe, the occipital lobe, the parietal lobe, or the frontal lobe of the brain, then administering to the human subject an anti-N3pGlu Aβ antibody.

170. The embodiment of any one of 167-169, wherein the human subject has tau burden in the posterolateral temporal lobe or the temporal lobe of the brain.

171. The embodiment of any one of 167-170, wherein the human subject has tau burden in the occipital lobe of the brain.

172. The embodiment of any one of 167-171, wherein the human subject has tau burden in the parietal lobe of the brain.

173. The embodiment of any one of 167-172, wherein the human subject has tau burden in the frontal lobe of the brain.

174. The embodiment of any one of 167-173, wherein the human subject has tau burden in the posterolateral temporal (PLT) and/or occipital lobe of the brain.

175. The embodiment of any one of 167-174, wherein the human subject has tau burden in i) parietal or precuneus region or ii) in frontal region along with tau burden in PLT or occipital regions of the brain.

176. The embodiment of any one of 167-175, wherein the human subject has tau burden i) isolated to frontal lobe or ii) in regions of the temporal lobe that do not include the posterolateral temporal region (PLT) of the brain.

177. The embodiment of any one of 167-176, wherein the human subject has tau burden in posterior-lateral temporal lobe, occipital lobe, and parietal lobe of the brain.

178. The embodiment of any one of 167-177, wherein the human subject has tau burden in posterior-lateral temporal lobe, occipital lobe, parietal lobe, and frontal lobe of the brain.

179. The embodiment of any one of 167-178, wherein the human subject has tau burden in posterior-lateral temporal lobe, occipital lobe, parietal lobe and/or frontal lobe of the brain.

180. The embodiment of any one of 167-179, wherein the patient has one or two alleles of APOE4.

181. The embodiment of any one of 1-27, wherein the human patient is further administered one or more effective doses of solanezumab or an antibody comprising portions of solanezumab.

182. The embodiment of 181, wherein the anti-N3pGlu Aβ antibody and solanezumab or the antibody comprising portions of solanezumab are administered simultaneously, separately, or sequentially.

183. The embodiment of any one of 28-55, wherein the human patient is further administered one or more effective doses of solanezumab or an antibody comprising portions of solanezumab.

184. The embodiment of 183, wherein the anti-N3pGlu Aβ antibody and solanezumab or the antibody comprising portions of solanezumab are administered simultaneously, separately, or sequentially.

185. The embodiment of any one of 56-82, wherein the human patient is further administered one or more effective doses of solanezumab or an antibody comprising portions of solanezumab.

186. The embodiment of 185, wherein the anti-N3pGlu Aβ antibody and solanezumab or the antibody comprising portions of solanezumab are administered simultaneously, separately, or sequentially.

187. The embodiment of any one of 83-110, wherein the human patient is further administered one or more effective doses of solanezumab or an antibody comprising portions of solanezumab.

188. The embodiment of 187, wherein the anti-N3pGlu Aβ antibody and solanezumab or the antibody comprising portions of solanezumab are administered simultaneously, separately, or sequentially.

189. The embodiment of any one of 111-126, wherein the human patient is further administered one or more effective doses of solanezumab or an antibody comprising portions of solanezumab.

190. The embodiment of 189, wherein the anti-N3pGlu Aβ antibody and solanezumab or the antibody comprising portions of solanezumab are administered simultaneously, separately, or sequentially.

191. The embodiment of any one of 127-166, wherein the human patient is further administered one or more effective doses of solanezumab or an antibody comprising portions of solanezumab.

192. The embodiment of 191, wherein the anti-N3pGlu Aβ antibody and solanezumab or the antibody comprising portions of solanezumab are administered simultaneously, separately, or sequentially.

193. The embodiment of any one of 167-180, wherein the human patient is further administered one or more effective doses of solanezumab or an antibody comprising portions of solanezumab.

194. The embodiment of 193, wherein the anti-N3pGlu Aβ antibody and solanezumab or the antibody comprising portions of solanezumab are administered simultaneously, separately, or sequentially.

195. The embodiment of any one of 181-194, wherein solanezumab or the antibody comprising portions of solanezumab is administered to the human subject to maintain amyloid beta levels within a normal range.

196. The embodiment of any one of 181-194, wherein solanezumab or the antibody comprising portions of solanezumab is administered to the human subject to prevent an increase in amyloid plaque levels.

197. The embodiment of any one of 181-194, wherein solanezumab or the antibody comprising portions of solanezumab is administered to the human subject to reduce the rate of increase of amyloid plaque levels.

SEQ ID NO: 1; Light Chain Variable Region (LCVR)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
SEQ ID NO: 2; Heavy Chain Variable Region (HCVR)
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSS
SEQ ID NO: 3; Light Chain (LC)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
SEQ ID NO: 4; Heavy Chain (HC)
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
SEQ ID NO: 5; Light Chain Complementarity Determining Region 1 (LCDR1)
KSSQSLLYSRGKTYLN
SEQ ID NO: 6; Light Chain Complementarity Determining Region 2 (LCDR2)
AVSKLDS
SEQ ID NO: 7; Light Chain Complementarity Determining Region 3 (LCDR3)
VQGTHYPFT
SEQ ID NO: 8; Heavy Chain Complementarity Determining Region 1 (HCDR1)
GYDFTRYYIN
SEQ ID NO: 9; Heavy Chain Complementarity Determining Region 2 (HCDR2)
WINPGSGNTKYNEKFKG
SEQ ID NO: 10; Heavy Chain Complementarity Determining Region 3 (HCDR3)
EGITVY
SEQ ID NO: 11; Nucleotide Sequence for SEQ ID NO: 1; Light Chain Variable Region
(LCVR)
GATATTGTGATGACTCAGACTCCACTCTCCCTGTCCGTCACCCCTGGACAGCC
GGCCTCCATCTCCTGCAAGTCAAGTCAGAGCCTCTTATATAGTCGCGGAAAAA
CCTATTTGAATTGGCTCCTGCAGAAGCCAGGCCAATCTCCACAGCTCCTAATT
TATGCGGTGTCTAAACTGGACTCTGGGGTCCCAGACAGATTCAGCGGCAGTG
GGTCAGGCACAGATTTCACACTGAAAATCAGCAGGGTGGAGGCCGAAGATGT
TGGGGTTTATTACTGCGTGCAAGGTACACATTACCCATTCACGTTTGGCCAAG
GGACCAAGCTGGAGATCAAA
SEQ ID NO. 12; Nucleotide Sequence for SEQ ID NO: 2; Heavy Chain Variable Region
(HCVR)
CAGGTGCAGCTGGTGCAGTCTGGGGCTGAGGTGAAGAAGCCTGGGTCCTCAG
TGAAGGTTTCCTGCAAGGCATCTGGTTACGACTTCACTAGATACTATATAAAC
TGGGTGCGACAGGCCCCTGGACAAGGGCTTGAGTGGATGGGATGGATTAATC
CTGGAAGCGGTAATACTAAGTACAATGAGAAATTCAAGGGCAGAGTCACCAT
TACCGCGGACGAATCCACGAGCACAGCCTACATGGAGCTGAGCAGCCTGAGA
TCTGAGGACACGGCCGTGTATTACTGTGCGAGAGAAGGCATCACGGTCTACT
GGGGCCAAGGGACCACGGTCACCGTCTCCTCA
SEQ ID NO. 13; Nucleotide Sequence for SEQ ID NO: 3; Light Chain (LC)
GATATTGTGATGACTCAGACTCCACTCTCCCTGTCCGTCACCCCTGGACAGCC
GGCCTCCATCTCCTGCAAGTCAAGTCAGAGCCTCTTATATAGTCGCGGAAAAA
CCTATTTGAATTGGCTCCTGCAGAAGCCAGGCCAATCTCCACAGCTCCTAATT
TATGCGGTGTCTAAACTGGACTCTGGGGTCCCAGACAGATTCAGCGGCAGTG
GGTCAGGCACAGATTTCACACTGAAAATCAGCAGGGTGGAGGCCGAAGATGT
TGGGGTTTATTACTGCGTGCAAGGTACACATTACCCATTCACGTTTGGCCAAG
GGACCAAGCTGGAGATCAAACGAACTGTGGCTGCACCATCTGTCTTCATCTTC
CCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTC
ACAAAGAGCTTCAACAGGGGAGAGTGC
SEQ ID NO. 14; Nucleotide Sequence for SEQ ID NO: 4; Heavy Chain (HC)
CAGGTGCAGCTGGTGCAGTCTGGGGCTGAGGTGAAGAAGCCTGGGTCCTCAG
TGAAGGTTTCCTGCAAGGCATCTGGTTACGACTTCACTAGATACTATATAAAC
TGGGTGCGACAGGCCCCTGGACAAGGGCTTGAGTGGATGGGATGGATTAATC
CTGGAAGCGGTAATACTAAGTACAATGAGAAATTCAAGGGCAGAGTCACCAT
TACCGCGGACGAATCCACGAGCACAGCCTACATGGAGCTGAGCAGCCTGAGA
TCTGAGGACACGGCCGTGTATTACTGTGCGAGAGAAGGCATCACGGTCTACT
GGGGCCAAGGGACCACGGTCACCGTCTCCTCAGCCTCCACCAAGGGCCCATC
GGTCTTCCCGCTAGCACCCTCCTCCAAGAGCACCTCTGGGGGCACAGCGGCCC
TGGGCTGCCTGGTCAAGGACTACTTCCCCGAACCGGTGACGGTGTCGTGGAA
CTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGG
ACAAGAAAGTTGAGCCCAAATCTTGTGACAAAACTCACACATGCCCACCGTG
CCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGACGAGCTGACCAAGAACCAGGTCAGCCTGACCTGCCT
GGTCAAAGGCTTCTATCCCAGCGACATCGCCGTGGAGTGGGAGAGCAATGGG
CAGCCGGAGAACAACTACAAGACCACGCCCCCCGTGCTGGACTCCGACGGCT
CCTTCTTCCTCTATAGCAAGCTCACCGTGGACAAGAGCAGGTGGCAGCAGGG
GAACGTCTTCTCATGCTCCGTGATGCATGAGGCTCTGCACAACCACTACACGC
AGAAGAGCCTCTCCCTGTCTCCGGGT
SEQ ID NO: 15; Amino acid sequence for the Light Chain of Solanezumab
DVVMTQSPLSLPVTLGQPASISCRSSQSLIYSDGNAYLHWFLQKPGQSPRLLIYKV
SNRFSGVPDRFSGSGSGTDFTLKISRVEAEDVGVYYCSQSTHVPWTFGQGTKVEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
SEQ ID NO: 16; Amino acid sequence for the Heavy Chain of Solanezumab
EVQLVESGGGLVQPGGSLRLSCAASGFTFSRYSMSWVRQAPGKGLELVAQINSV
GNSTYYPDTVKGRFTISRDNAKNTLYLQMNSLRAEDTAVYYCASGDYWGQGTL
VTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSL
SLSPGK

We claim:

1. A method to reduce amyloid beta (Aβ) plaques in the brain of a human Alzheimer's Disease (AD) subject comprising:
administering to the subject three first doses of 700 mg of an anti-N3pG Aβ antibody, wherein each first dose is administered at a frequency of once every four weeks; and
four weeks after administration of the three first doses, administering to the subject one or more second doses of 1400 mg of the anti-N3pG Aβ antibody at a frequency of once every four weeks;
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

2. The method of claim 1, wherein the anti-N3pG Aβ antibody is administered until the Aβ plaques are cleared.

3. The method of claim 1, wherein the anti-N3pG Aβ antibody is administered until at least one of:
i) the Aβ plaques in the subject are 25 centiloids or lower as measured by two consecutive amyloid PET imaging scans, wherein the two consecutive amyloid PET imaging scans are at least 6 months apart, or
ii) the Aβ plaques in the subject are 11 centiloids or lower as measured by a single amyloid PET imaging scan.

4. The method of claim 1, wherein the anti-N3pG Aβ antibody is administered until the subject is amyloid negative.

5. The method of claim 1, wherein the anti-N3pGlu Aβ antibody is administered until the Aβ plaque level of <24.1 CL is reached as measured by amyloid PET imaging scan.

6. The method of any one of claims 1 to 5, wherein the anti-N3pG Aβ antibody doses are administered over a period of no more than 72 weeks.

7. The method of claim 1, further comprising a step of evaluating magnetic resonance image (MRI) scan of the subject's brain for amyloid-related imaging abnormality (ARIA), after the administration of the three first doses and modifying one or more of the administration steps until ARIA-E has resolved.

8. The method of any one of claims 1-7, wherein administration of the anti-N3pGlu Aβ antibody is temporarily withheld or discontinued if symptoms consistent with ARIA occur.

9. The method of any one of claims 1-8, wherein administration of the anti-N3pGlu Aβ antibody is temporarily withheld if symptoms consistent with mild to moderate ARIA occur.

10. The method of any one of claims 1-8, wherein administration of the anti-N3pGlu Aβ antibody is discontinued if symptoms consistent with severe or symptomatic ARIA occur.

11. The method of claim 1, wherein administration of the anti-N3pGlu Aβ antibody:
a) slows disease progression by at least 15% as compared to being untreated estimated by Disease Progression Model (DPM), wherein disease progression is measured by iADRS or CDR-SB;
b) slows disease progression by at least 15% as compared to being untreated estimated by a mixed-model repeated-measures analysis (MMRM), wherein disease progression is measured by iADRS or CDR-SB;
c) slows disease progression by at least 15% as compared to being untreated, wherein disease progression is measured by Integrated Alzheimer's Disease Rating Scale (iADRS);
d) slows disease progression by at least 3 as compared to being untreated, wherein disease progression is measured by Integrated Alzheimer's Disease Rating Scale (iADRS);
e) slows disease progression by at least 20% as compared to being untreated, wherein the disease progression is measured by Clinical Dementia Rating Scale—Sum of Boxes (CDR-SB);
f) reduces the level of Aβ plaque in the brain of the subject by at least 40% as measured by amyloid PET imaging;
g) slows tau accumulation in the frontal lobe by at least 50% as compared to being untreated;
h) limits an increase in the subject's frontal lobe tau over 72 weeks to less than 0.04 SUVr (standardized uptake value ratio) as measured by tau PET imaging;
i) reduces plasma P-tau 217 by at least 5% from baseline; or
j) reduces glial fibrillary acidic protein (GFAP) by at least 5% from baseline.

12. The method of claim 1, wherein administering the anti-N3pGlu Aβ antibody reduces Aβ plaques by about an average of about 50 centiloids to about 100 centiloids as compared to Aβ plaques prior to administering the one or more first doses, wherein the Aβ plaques are measured by amyloid PET imaging scan.

13. The method of claim 1, wherein the subject has a brain tau level of less than 1.46 standardized uptake value ratio (SUVr) prior to administering the anti-N3pGlu Aβ antibody, wherein the brain tau level is measured by tau PET imaging scan.

14. The method of claim 1, wherein the subject has a brain tau level of greater than 1.10 SUVr and less than 1.46 SUVr prior to administering the anti-N3pGlu Aβ antibody, wherein the brain tau level is measured by tau PET imaging scan.

15. The method of claim 13 or claim 14, wherein brain tau level is measured by ¹⁸F-flortaucipir PET imaging.

16. The method of claim 1, wherein the subject has a negative tau PET imaging scan in a frontal lobe brain region prior to administering the anti-N3pGlu Aβ antibody.

17. The method of claim 1, wherein 24 weeks of administering the anti-N3pGlu Aβ antibody reduces the Aβ plaque by at least 60%.

18. The method of claim 1, wherein administering the anti-N3pGlu Aβ antibody comprises administering each dose of the anti-N3pGlu Aβ antibody intravenously at a concentration of 4 mg/mL to 10 mg/mL over at least 30 minutes.

19. The method of claim 1, wherein the subject has a baseline MMSE (Mini-Mental State Exam) score of 20 to 28 prior to administering the anti-N3pGlu Aβ antibody.

20. The method of claim 1, wherein administering the anti-N3pGlu Aβ antibody does not reduce the subject's hippocampal volume during the course of the administration.

21. The method of claim 1, wherein the subject has early symptomatic Alzheimer's Disease prior to administering the anti-N3pGlu Aβ antibody.

22. The method of claim 1, wherein the subject has at least one APOE4 allele.

23. The method of claim 1, wherein the level of Aβ plaques in the brain of the subject is sustained at normal levels for at least 52 weeks after completing administration of the second dose.

24. The method of claim 1, wherein administration of the anti-N3pGlu Aβ antibody is stopped if the Aβ plaques in the brain of the subject reach normal levels by 24 weeks or the Aβ plaques level in the brain of the subject stop reducing.

25. The method of claim 20, wherein the level of Aβ plaques in the brain of the subject is sustained at normal levels for at least 52 weeks after the administration of the anti-N3pGlu Aβ antibody is stopped.

26. The method of claim 1, wherein administering the anti-N3pGlu Aβ antibody reduces the level of Aβ plaques in the brain of the subject to normal levels by 24 weeks.

27. The method of claim 24, wherein the level of Aβ plaques in the brain of the subject is sustained at normal levels for at least 52 additional weeks.

28. The method of any one of claims 23-27, wherein the subject has an increase in tau level in a parietal lobe of less than 0.06 SUVr 76 weeks after the administration of the anti-N3pGlu Aβ antibody, wherein the brain tau level is measured by tau PET imaging scan.

29. The method of claim any one of claims 23-28, wherein the subject has a brain tau level of less than 0.4 SUVr in a frontal lobe region 76 weeks after the administration of the anti-N3pGlu Aβ antibody, wherein the brain tau level is measured by tau PET imaging scan.

30. A method of slowing disease progression in a human Alzheimer's Disease subject, comprising:
administering to the subject an anti-N3pGlu Aβ antibody to slow disease progression by at least 15% as measured by Integrated Alzheimer's Disease Rating Scale (iADRS), the administering comprising:
i) administering to the subject three first doses of 700 mg of the anti-N3pGlu Aβ antibody, wherein each first dose is administered at a frequency of once every four weeks; and
ii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

31. A method of slowing disease progression in a human Alzheimer's Disease subject, comprising:
administering to the subject an anti-N3pGlu Aβ antibody to slow disease progression by at least 20% as measured by Clinical Dementia Rating Scale—Sum of Boxes (CDR-SB), the administering comprising:
i) administering to the subject three first doses of 700 mg of the anti-N3pGlu Aβ antibody, wherein each first dose is administered at a frequency of once every four weeks; and
ii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

32. The method of claim 30 or claim 31, wherein the anti-N3pG Aβ antibody is administered until the Aβ plaques are cleared.

33. The method of any one of claims 30 to 32, wherein the anti-N3pG Aβ antibody is administered until at least one of:
i) the Aβ plaques in the subject are 25 centiloids or lower as measured by two consecutive amyloid PET imaging scans, wherein the two consecutive amyloid PET imaging scans are at least 6 months apart, and
ii) the Aβ plaques in the subject are 11 centiloids or lower as measured by a single PET amyloid imaging scan.

34. The method of claim 30 or claim 31, wherein the anti-N3pG Aβ antibody is administered until the subject is amyloid negative.

35. The method of claim 30 or claim 31, wherein the subject is amyloid negative when the amyloid plaque level of <24.1 CL is reached as measured by amyloid PET imaging scan.

36. The method of any one of claims 30 to 35, wherein the anti-N3pG Aβ antibody is administered over a period of no more than 72 weeks.

37. The method of claim 30 or claim 31, further comprising a step of evaluating magnetic resonance image (MRI) scan of the subject's brain for amyloid-related imaging abnormality (ARIA), after the administration of the three first doses and modifying one or more of the administration steps until ARIA-E is resolved.

38. The method of any one of claims 30-37, wherein administration of the anti-N3pGlu Aβ antibody is withheld or discontinued if symptoms consistent with ARIA occur.

39. The method of any one of claims 30-38, wherein administration of the anti-N3pGlu Aβ antibody is temporarily withheld if symptoms consistent with mild to moderate ARIA occur.

40. The method of any one of claims 30-38, wherein administration of the anti-N3pGlu Aβ antibody is discontinued if symptoms consistent with severe or symptomatic ARIA occur.

41. The method of claim 30 or claim 31, wherein administering the anti-N3pGlu Aβ antibody reduces Aβ plaques by about an average of about 50 centiloids to about 100 centiloids as compared to Aβ plaques prior to administering the one or more first doses, wherein the Aβ plaques are measured by amyloid PET imaging scan.

42. The method of claim 30 or claim 31, wherein the subject has a brain tau level of less than 1.46 standardized uptake value ratio (SUVr) prior to administering the anti-N3pGlu Aβ antibody, wherein the brain tau level is measured by tau PET imaging scan.

43. The method of claim 30 or claim 31, wherein the subject has a brain tau level of greater than 1.10 SUVr and less than 1.46 SUVr prior to administering the anti-N3pGlu Aβ antibody, wherein the brain tau level is measured by tau PET imaging scan.

44. The method of claim 30 or claim 31, wherein the subject has a negative tau PET imaging scan in a frontal lobe brain region prior to administering the anti-N3pGlu Aβ antibody.

45. The method of claim 30 or claim 31, wherein 24 weeks of administering the anti-N3pGlu Aβ antibody reduces the Aβ plaque by at least 60%.

46. The method of claim 30 or claim 31, wherein administering the anti-N3pGlu Aβ antibody comprises administering each dose of the anti-N3pGlu Aβ antibody intravenously at a concentration of 4 mg/mL to 10 mg/mL over at least 30 minutes.

47. The method of claim 30 or claim 31, wherein the subject has a baseline MMSE (Mini-Mental State Exam) score of 20 to 28 prior to administering the anti-N3pGlu Aβ antibody.

48. The method of claim 30 or claim 31, wherein administering the anti-N3pGlu Aβ antibody does not reduce the subject's hippocampal volume during the course of the administration.

49. The method of claim 30 or claim 31, wherein the subject has early symptomatic Alzheimer's Disease prior to administering the anti-N3pGlu Aβ antibody.

50. The method of claim 30 or claim 31, wherein the subject has at least one APOE4 allele.

51. The method of claim 30 or claim 31, wherein the level of Aβ plaques in the brain of the subject is sustained at normal levels for at least 52 weeks after completing administration of the second dose.

52. The method of claim 30 or claim 31, wherein administration of the anti-N3pGlu Aβ antibody is stopped if the Aβ plaques in the brain of the subject reach normal levels by 24 weeks or the Aβ plaques stop reducing.

53. The method of claim 52, wherein the level of Aβ plaques in the brain of the subject is sustained at normal levels for at least 52 weeks after the administration of the anti-N3pGlu Aβ antibody is stopped.

54. The method of claim 30 or claim 31, wherein administering the anti-N3pGlu Aβ antibody reduces the level of Aβ plaques in the brain of the subject to normal levels by 24 weeks.

55. The method of claim 54, wherein the level of Aβ plaques in the brain of the subject is sustained at normal levels for at least 52 additional weeks.

56. The method of any one of claims 51 to 55, wherein the subject has an increase in tau level in a parietal lobe of less than 0.06 SUVr 76 weeks after the administration of the anti-N3pGlu Aβ antibody, wherein the brain tau level is measured by tau PET imaging scan.

57. The method of any one of claims 51 to 56, wherein the subject has a brain tau level of less than 0.4 SUVr in a frontal lobe region 76 weeks after the administration of the anti-N3pGlu Aβ antibody, wherein the brain tau level is measured by tau PET imaging scan.

58. A method of treating Alzheimer's Disease in a subject in need thereof comprising:
i) administering to the subject three first doses of 700 mg of the anti-N3pGlu Aβ antibody, wherein each first dose is administered at a frequency of once every four weeks; and
ii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

59. An improved method of treating Alzheimer's Disease in a subject in need thereof comprising:
i) administering to the subject three first doses of 700 mg of the anti-N3pGlu Aβ antibody, wherein each first dose is administered at a frequency of once every four weeks; and
ii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

60. A method of treating Alzheimer's Disease in a subject in need thereof comprising:
i) administering to the subject three first doses of 700 mg of the anti-N3pGlu Aβ antibody, wherein each first dose is administered at a frequency of once every four weeks;
ii) evaluating magnetic resonance image (MRI) scan of the subject's brain for amyloid-related imaging abnormality (ARIA), after the administration of the three first doses and prior to the administration of the one or more second doses wherein the administration of one or more second doses is temporarily withheld if symptoms consistent with ARIA occur;
iii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

61. The method of claim 60, wherein the administration of one or more second doses is re-initiated after resolution of ARIA symptoms or radiographic stabilization on MRI.

62. The method of claim 60 or 61, wherein the one or more second doses are withheld, and corticosteroids are administered to the subject.

63. A method of treating Alzheimer's Disease in a subject in need thereof comprising:
i) administering to the subject three first doses of 700 mg of the anti-N3pGlu Aβ antibody, wherein each first dose is administered at a frequency of once every four weeks;
ii) evaluating magnetic resonance image (MRI) scan of the subject's brain for amyloid-related imaging abnormality (ARIA), after the administration of the three first doses and prior to the administration of the one or more second doses wherein the administration of one or more second doses is discontinued if symptoms consistent with severe or symptomatic ARIA occur;
iii) four weeks after administration of the three first doses, administering one or more second doses of 1400 mg of the anti-N3pGlu Aβ antibody at a frequency of once every four weeks; and
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

64. The method of claim 63, wherein the administration of one or more second doses is discontinued, and corticosteroids are administered to the subject.

65. A method of treating Alzheimer's Disease in a subject in need thereof until symptoms consistent with ARIA-E occur comprising:
i) administering to the subject three first doses of 700 mg of the anti-N3pGlu Aβ antibody, wherein each first dose is administered at a frequency of once every four weeks; and
ii) four weeks after administration of the three first doses, administering one or more second doses of 1400 mg of the anti-N3pGlu Aβ antibody at a frequency of once every four weeks;
wherein the anti-N3pGlu Aβ antibody comprises a light chain variable region (LCVR) and a heavy chain variable region (HCVR), wherein the LCVR consists of the amino acid sequence of SEQ ID NO: 1 and the HCVR consists of the amino acid sequence of SEQ ID NO: 2.

66. The method of claim 65, wherein the symptoms of ARIA are detected by MRI or are presented in the subject.

67. A method for treating a patient with donanemab, wherein the patient is suffering from Alzheimer's disease, the method comprising the steps of:
a) administering [or having administered] 700 mg of donanemab every four weeks for the first three doses;
b) determining whether the patient has symptoms of ARIA-E i) by performing or having performed an MRI prior to dose increase or ii) if clinical symptoms consistent with ARIA-E occur; and
c) if the patient has moderate symptoms of ARIA-E, temporarily discontinuing treatment with donanemab; and
d) if the patient does not have symptomatic ARIA-E, administering donanemab to the patient in an amount of 1400 mg every four weeks until brain amyloid is cleared, is negative, or is <24.1 CL.

68. A method for treating a patient with donanemab, wherein the patient is suffering from Alzheimer's disease, the method comprising the steps of:
a) administering [or having administered] 700 mg of donanemab every four weeks for the first three doses;
b) determining whether the patient has symptoms of ARIA-E i) by performing or having performed an MRI prior to dose increase or ii) if clinical symptoms consistent with ARIA-E occur; and if the patient does not have symptomatic ARIA-E, administering donanemab to the patient in an amount of 1400 mg every four weeks until brain amyloid is cleared, is negative, or is <24.1 CL.

69. An improved method for treating a patient with donanemab to a patient suffering from Alzheimer's disease, wherein the improvement comprises:
a) administering [or having administered] 700 mg of donanemab every four weeks for the first three doses;
b) determining whether the patient has symptoms of ARIA-E i) by performing or having performed an MRI prior to dose increase or ii) if clinical symptoms consistent with ARIA-E occur; and
c) if the patient has moderate symptoms of ARIA-E, temporarily discontinuing treatment with donanemab; and
d) if the patient does not have symptomatic ARIA-E, internally administering donanemab to the patient in an amount of 1400 mg every four weeks until brain amyloid is cleared, is negative, or is <24.1 CL.

70. An improved method for treating a patient with donanemab to a patient suffering from Alzheimer's disease, wherein the improvement comprises:
a) administering [or having administered] 700 mg of donanemab every four weeks for the first three doses;
b) determining whether the patient has symptoms of ARIA-E i) by performing or having performed an MRI prior to dose increase or ii) if clinical symptoms consistent with ARIA-E occur; and if the patient does not have symptomatic ARIA-E, internally administering donanemab to the patient in an amount of 1400 mg every four weeks until brain amyloid is cleared, is negative, or is <24.1 CL.

71. A method for treating a patient with donanemab, wherein the patient is suffering from Alzheimer's Disease, the method comprising the steps of:
a) administering [or having administered] 700 mg of donanemab every four weeks for the first three doses;
b) discontinuing treatment if the patient has moderate symptoms of ARIA-E; and
c) continuing treatment once ARIA-E resolves by administering donanemab to the patient in an amount of 1400 mg every four weeks until brain amyloid is cleared, is negative, is <24.1 CL, or ARIA-E symptoms reappear.

72. The method of claim 71, wherein the symptoms or ARIA-E are confirmed or are determined by an MRI scan.

73. A method for treating a patient with donanemab, wherein the patient is suffering from Alzheimer's disease, the method comprising the steps of:
a) administering [or having administered] 700 mg of donanemab every four weeks for the first three doses;
b) administering donanemab to the patient in an amount of 1400 mg every four weeks until brain amyloid is cleared, is negative, or is ≤24.1 CL so long as the patient does not have symptomatic ARIA-E.

74. The method of claim 73, wherein the symptoms or ARIA-E are confirmed or are determined by an MRI scan.
